(19) United States
(12) Patent Application Publication
(10) Pub. No.: US 2004/0038861 A1
(43) Pub. Date: Feb. 26, 2004

(54) Methods and compositions for normalizing lipid levels in mammalian tissues

(76) Inventors: Garth J. S. Cooper, Auckland (NZ); Kerry M. Loomes, Auckland (NZ); Rachel N. Watson, Auckland (NZ)

Correspondence Address: BUCHANAN INGERSOLL, P.C., ONE OXFORD CENTRE, 301 GRANT STREET, 20TH FLOOR, PITTSBURGH, PA, 15219, US

(21) Appl. No.: 10/306,645

(22) Filed: Nov. 26, 2002

Related U.S. Application Data
(60) Provisional application No. 60/333,422, filed on Nov. 26, 2001.

(51) Int. Cl. A61K 38/17

(52) U.S. Cl. 514/2

(57) ABSTRACT

The invention provides methods of stimulating lipolysis in a mammalian cells and tissues by contacting the cell or tissue with an agonist of a CGRP receptor such as the high affinity CGRP receptor. The agonist may preferentially stimulate the high affinity CGRP receptor compared to the metabolic amylin receptor. Methods for screening for identifying receptor agonists are provided, pharmaceutical compositions comprising such agonists, and therapeutic regimens using such agonists are provided.

### CROSS-REFERENCE TO RELATED APPLICATIONS

This application claims benefit of U.S. provisional patent application No. 60/333,422, filed Nov. 26, 2001, which is incorporated herein by reference.

### FIELD OF THE INVENTION

This invention provides methods and compositions for modulating lipid levels in a cell or tissue, and for identifying agents useful for modulating lipid levels. The invention finds use in the fields of biology and medicine.

### BACKGROUND

Disorders of lipid metabolism play roles in a variety of diseases and conditions. For example, there is strong evidence that accumulation of lipid in skeletal muscle is a key metabolic abnormality underlying the development of insulin resistance, not only in muscle itself but also in liver, adipose, and other tissues. McGarry et al. What if Minkowski had been ageusic? An alternative angle on diabetes. Science. 1992 October 30;258(5083):766-70; Ellis et al. Long-chain acyl-CoA esters as indicators of lipid metabolism and insulin sensitivity in rat and human muscle. Am J Physiol Endocrinol Metab. 2000 September;279(3):E554-60; Furler et al. A high-fat diet influences insulin-stimulated posttransport muscle glucose metabolism in rats. Metabolism. 1997 September;46(9):1101-6; Dobbins et al. Prolonged inhibition of muscle carnitine palmitoyltransferase-1 promotes intramyocellular lipid accumulation and insulin resistance in rats. Diabetes. 2001 Janarary;50(1):123-30; Kraegen et al. Development of muscle insulin resistance after liver insulin resistance in high-fat-fed rats. Diabetes. 1991 Novermber;40(11):1397-403; Unger et al. Lipotoxic diseases of nonadipose tissues in obesity. Int J Obes Relat Metab Disord. 2000 November;24 Suppl 4:S28-32; Kelley et al. Skeletal musclefatty acid metabolism in association with insulin resistance, obesity, and weight loss. Am J Physiol. 1999 December;277(6 Pt 1):E1130-41; Simoneau et al. Markers of capacity to utilizefrttty acids in human skeletal muscle. relation to insulin resistance and obesity and effects of weight loss. FASEB J. 1999 November;13(14):2051-60; Manco et al. Insulin resistance directly correlates with increased saturatedfatty acids in skeletal muscle triglycerides. Metabolism. 2000 Febuary;49(2):220-4.

Insulin resistance is a primary pathogenic process for several major metabolic diseases of humans with common underlying pathogenic mechanisms, referred to as the “metabolic syndrome” or “Syndrome X complex.” Diseases commonly included within Syndrome X complex include type-2 diabetes mellitus, hypertension, obesity, dyslipidaemia, atherosclerosis, and thrombosis (see, e.g., Zierath et al., 2000, Diabetologia 43:821-35; Bergman et al., 2001, J Investig Med. 49:119-26; Olefsky et al., 1995, Am J Clin Nutr. 61(4 Suppl):980S-986S; Ginsberg et al., 2000, J Clin Invest. 106:453-8; Groop et al., 1999, Diabetes Obes Metab. 1 Suppl 1:S1-7; Fujimoto et al., 2000, Am J Med. 17;108 Suppl 6a:9S-14S; Ferrannini et al., 1986, N Engl J Med., 1986, 317:350-7; Landsberg et al., 1999, Clin Exp Hypertens; 21:885-894; Ferrannini et al., 1995, Metabolism, 44 (9 Suppl 3):15-7; Kahn et al., 2000, J Clin Invest. 106:473-81; Ginsberg et al., 2000, J Cardiovasc Risk 7:325-31; Howard et al., 1999, Am J Cardiol. 8;84(1A):28J-32J; Grundy et al., 1999, Am J Cardiol. 13;83(9B):25F-29F; Misra et al., 2000, J Cardiovasc Risk. 7(3):215-29).

It would be desirable to develop pharmaceuticals and treatment strategies to normalize the lipid content in tissues. For example, such pharmaceuticals and treatment strategies would be useful to treat insulin resistance.

### SUMMARY OF THE INVENTION

In one aspect, the invention provides a method of stimulating lipolysis in a tissue of a mammal (e.g., skeletal muscle or liver) by contacting the tissue with an agonist of the high affinity CGRP receptor in an amount of agonist effective to preferentially stimulate activity of a high affinity CGRP receptor compared to the metabolic amylin (e.g., stimulate activity of a high affinity CGRP receptor without substantially stimulating activity of a metabolic amylin receptor). In an embodiment the agonist is CGRP-1. In an embodiment, the tissue is contacted with CGRP-1 at between about 10⁻¹⁵ M and about 10⁻¹⁰ M. In an embodiment, the amount is at less than 300 pM. In an embodiment, the tissue is isolated. In a further aspect, the method includes the step of detecting stimulation of lipolysis in the tissue.

In one aspect, the invention provides a method of stimulating lipolysis in a mammalian cell, such as a skeletal muscle cell, by contacting the cell with a CGRP polypeptide or biologically functional variant thereof. In an embodiment, the CGRP polypeptide has the sequence of a naturally occurring CGRP polypeptide, such as a human CGRP. In an embodiment, the CGRP polypeptide is a human CGRP-1 polypeptide, or biologically functional variant thereof. In an embodiment, the polypeptide or variant preferentially stimulates the high affinity CGRP receptor.

In an aspect the invention provides a method of stimulating lipolysis in skeletal muscle or liver of a mammal by contacting the skeletal muscle or liver with CGRP-1 or a metabolic receptor stimulating variant thereof. The method may further comprise monitoring a change in the level of lipolysis in a tissue of the mammal (e.g., by measuring the amount of free fatty acids in muscle, liver, blood or other tissue of the mammal).

In an aspect, the invention provides a therapeutic regimen including (i) administering a CGRP-1 polypeptide, a biologically function variant thereof, or a metabolic receptor stimulating variant thereof to a mammal suffering from or susceptible to a condition characterized by accumulation of lipid in skeletal muscle, and (ii) monitoring lipolysis in the mammal.

In an aspect, the invention provides a method of reducing skeletal muscle lipid levels in a mammal in need of such treatment by administering an agonist of the high affinity CGRP receptor under conditions in which the metabolic amylin receptor is not stimulated by the agonist or the high affinity receptor is preferentially stimulated, wherein the mammal has a condition or disease characterized by lipid accumulation in a tissue. In an embodiment, the mammal is insulin resistant. In an embodiment, the method further includes detecting a change in the amount of free fatty acids in a tissue (e.g., skeletal muscle, liver or blood) of the mammal. In an embodiment, the method further includes monitoring the course of the condition or disease. In an embodiment, the administration of the agonist does not result in an increase in blood glucose levels in the mammal.

In a related aspect, the invention provides a method of reducing lipid levels in a tissue (e.g., skeletal muscle or liver) by contacting the tissue with a CGRP polypeptide or biologically functional variant thereof. In an embodiment, the CGRP polypeptide or variant is administered to a mammal in an amount effective to reduce tissue lipid levels. In an embodiment, the mammal is insulin resistant and the polypeptide or variant is administered in an amount effective to reduce insulin resistance in the mammal. In an embodiment, the polypeptide or variant preferentially stimulates the high affinity CGRP receptor.

In one aspect, the invention provides use of a CGRP polypeptide, such as CGRP-1, or a biologically functional variant thereof in the preparation of a medicament for reducing lipid levels in skeletal muscle of a mammal or in the preparation of a medicament for treatment of insulin resistance.

In one aspect, the invention provides a method of stimulating lipolysis in a cell that expresses a high affinity CGRP receptor, by contacting the cell with an agonist of the high affinity CGRP receptor. In an embodiment, the agonist preferentially activates the high affinity CGRP receptor compared to the metabolic amylin receptor.

In a related aspect, the invention provides a method of stimulating lipolysis in a mammal by administering to the mammal an agonist of the high affinity CGRP receptor. In an embodiment, the agonist preferentially activates the high affinity CGRP receptor compared to the metabolic amylin receptor. In an embodiment, the agonist is a polypeptide, such as a CGRP polypeptide or biologically functional variant thereof. In various embodiments, the method includes measuring the amount of free fatty acids in a tissue (skeletal muscle, liver, serum, plasma or blood) of the mammal or monitoring the effect of the administration of CGRP to the mammal on insulin resistance in the mammal. In an embodiment, the agonist is administered in an amount effective to stimulate lipolysis without stimulating vasodilatation. In a related aspect, the invention provides a method of stimulating lipolysis in a tissue (e.g., skeletal muscle or liver) in a mammal in need of such treatment, by administering an amount of an agonist of the high affinity CGRP receptor sufficient to preferentially stimulate the high affinity CGRP receptor compared to the metabolic amylin receptor.

In various embodiments, the mammal to which an agonist is administered has a disease or condition characterized by lipid accumulation in skeletal muscle (e.g., insulin resistance, Syndrome X, type-2 diabetes) or in liver (e.g., hepatic steatosis or “fatty liver”).

In an embodiment of the invention, the agonist is administered with a pharmaceutically acceptable carrier. The invention also provides the use of an agonist of the high affinity CGRP receptor (such as a CGRP-1 polypeptide) for the preparation of a medicament for treatment of a condition or disease (e.g., insulin resistance or Syndrome X) characterized by lipid accumulation in a tissue (e.g., skeletal muscle or liver) of a mammal. In a related aspect, the invention provides the use of CGRP-1 in the preparation of a medicament for treating a mammal suffering from or susceptible to a condition characterized by accumulation of lipid in a tissue (e.g., skeletal muscle or liver), where the medicament when administered to the mammal results in a level of agonist in blood that is less than 300 pM or when administered to the mammal results in a level of agonist in blood that is less between about 10⁻¹⁵ M and about 10⁻¹⁰ M. In an aspect, the invention provides the use of CGRP-1 in the preparation of a medicament for treating a mammal suffering from or susceptible to a condition characterized by accumulation of lipid in a tissue (e.g., skeletal muscle or liver), where the medicament when administered to the mammal results in a level of agonist in blood that is between about 10⁻¹⁵ M and about 10⁻¹⁰ M. In an embodiment the condition is diabetes, insulin resistance, or Syndrome X.

The invention also provides methods of determining whether an agent useful for stimulating lipolysis in a mammal by determining whether the agent is an agonist of a CGRP receptor, such as the high affinity CGRP receptor. In an embodiment, the method further includes determining that the agent preferentially stimulates the high affinity CGRP receptor compared to the metabolic amylin receptor. In an embodiment, the method further includes comparing the EC₅₀ of the agent for effecting a response mediated by the metabolic amylin receptor (e.g., an effect on carbohydrate metabolism) and the EC₅₀ of the agent for effecting a response mediated by the high affinity CGRP receptor (an increase in free fatty acids in skeletal muscle tissue or a skeletal muscle cell). In an embodiment, the EC₅₀ values are determined in vitro using isolated skeletal muscle. In an embodiment, the effect carbohydrate metabolism is a change in tissue or serum glucose and/or lactate levels and/or tissue glycogen levels.

The invention also provides a method of determining whether a compound is useful as a therapeutic agent by determining whether an agent stimulates lipolysis in a mammalian tissue that expresses the high affinity CGRP receptor and the metabolic amylin receptor; and determining whether a lipolysis stimulating agent preferentially stimulates the high affinity CGRP receptor compared to the metabolic amylin receptor, where an agent that preferentially stimulates the high affinity CGRP receptor is determined to be useful as a therapeutic agent. This and other methods of the invention can be used to screen for compounds useful as therapeutic drug. In one embodiment, drug screening includes carrying out the aforementioned method with at least 25 different agents (e.g., simultaneously or sequentially).

In one embodiment, the invention provides a screening method that involves (i) identifying a plurality of agents that bind the high affinity CGRP receptor; and, (ii) selecting an agent from (i) that preferentially stimulates the high affinity CGRP receptor compared to the metabolic amylin receptor. In one embodiment, the method involves (i) identifying a plurality of agents that bind the high affinity CGRP receptor; and, (ii) selecting an agent from (i) that has an EC₅₀ for effecting a response mediated by the metabolic amylin receptor which is higher than the EC₅₀ of the agent for effecting a response mediated by the high affinity CGRP receptor. In an embodiment, the EC₅₀ values are determined in vitro using isolated skeletal muscle (e.g., from rat). In one embodiment, the EC₅₀ for effecting a response mediated by the metabolic amylin receptor is at least 10-fold higher than the EC₅₀ of the agent for effecting a response mediated by the high affinity CGRP receptor. The response mediated by the high affinity receptor can be an increase in free fatty acids in skeletal muscle tissue or a skeletal muscle cell and the response mediated by the metabolic amylin receptor can be an effect on carbohydrate metabolism.

In a related embodiment, the invention provides a method of determining whether an agent useful for stimulating lipolysis in a mammal by (i) identifying a plurality of agents that stimulate lipolysis in a tissue expressing the high affinity CGRP receptor; and, (ii) selecting an agent from (i) that preferentially stimulates the high affinity CGRP receptor compared to the metabolic amylin receptor. In an embodiment, the tissue is skeletal muscle (e.g., from rat). In an embodiment, the library screened has more than about 100 different test agents.

The invention also provides a method for determining whether an agent is useful for stimulating lipolysis in a mammal comprising comparing the lipolytic activity of the agent with the lipolytic activity of CGRP.

The invention also provides a method of treating insulin resistance in a mammal by administering a CGRP receptor agonist in an amount effective to activate a high affinity CGRP receptor in a tissue of the mammal without activating the metabolic amylin receptor, where the agonist is an agent identified using a screening method described above. In an embodiment, the treatment method includes the additional step of measuring the amount of free fatty acids in a tissue of the mammal or of monitoring insulin resistance in the mammal.

In an aspect, the invention provides a method of determining a dose or formulation of an agonist of the high affinity CGRP receptor that stimulates lipolysis in a tissue (e.g., skeletal muscle or liver) of a mammal while minimizing undesired side-effects in the mammal (e.g., an increase in blood glucose levels in the mammal or stimulation of vasodilatation in the mammal) by (i) conducting dose-response assays by (a) administering a plurality of different doses or formulations of the CGRP receptor agonist to test mammals; and (b) measuring the effect of each dose or formulation on lipolysis in a tissue of the test mammal and measuring the effect of each dose on the side-effect, thereby creating dose-response data for lipolysis and the side-effect; and, (ii) determining from the dose-response data a dose of the CGRP receptor agonist formulation that increases lipolysis but does not elicit the side-effect or for which the there is a substantial increase in lipolysis and a minimal increase in side-effects. In an embodiment, the effect of each dose or formulation on lipolysis is determined by measuring free fatty acid levels in a tissue of the animal.

The invention also provides a composition containing a therapeutically effective amount of an agent that preferentially stimulates the high affinity CGRP receptor compared the metabolic amylin receptor, and pharmaceutically acceptable excipient. In an embodiment, the composition, when administered to a mammal, stimulates the high affinity CGRP receptor in the mammal without stimulating the metabolic amylin receptor. In an embodiment, the composition stimulates lipolysis while minimally stimulating vasodilatation.

In an aspect, the invention provides a pharmaceutical composition in unit dosage form for administration to a mammal, said unit dosage including an agonist of the high affinity CGRP receptor in an amount sufficient result in a level of agonist in blood sufficient to preferentially stimulate activity of a high affinity CGRP receptor compared to the metabolic amylin (e.g., stimulate activity of a high affinity CGRP receptor without substantially stimulating activity of a metabolic amylin receptor. In an embodiment the agonist is CGRP-1) and a pharmaceutically acceptable excipient. In an embodiment, the agonist is CGRP-1 or a biologically functional variant thereof. In an embodiment the agonist is CGRP-1 and the level of agonist in blood is less than 300 pM. In an embodiment the agonist is CGRP-1 and the level of agonist in blood is between about 10⁻¹⁵ M and about 10⁻¹⁰ M.

The invention further provides the use of an agonist of the high affinity CGRP-1 receptor in the preparation of a medicament for treating a mammal suffering from or susceptible to a condition characterized by accumulation of lipid in a tissue (e.g., skeletal muscle or liver), wherein the medicament when administered to the mammal results in a level of agonist in the mammal that is sufficient to preferentially stimulate activity of a high affinity CGRP receptor compared to the metabolic amylin (e.g., stimulate activity of a high affinity CGRP receptor without substantially stimulating activity of a metabolic amylin receptor). In an embodiment the agonist is CGRP-1).

In another aspect, the invention provides a method of inhibiting lipolysis in a mammal by administering to the mammal an antagonist of the metabolic amylin receptor and/or high affinity CGRP metabolic amylin receptor (e.g., ^{8,37}amylin or ^{8,37}CGRP) in an amount effective to inhibit lipolysis.

In another aspect, the invention provides a method of stimulating lipolysis in a tissue of a mammal (e.g., skeletal muscle) by contacting the tissue with an agonist of the metabolic amylin receptor (e.g., CGRP).

### BRIEF DESCRIPTION OF THE FIGURES

FIG. 1 shows amino acid sequences of peptides used in the study. Peptides used rat amylin, rat CGRP-1 (CGRPα), rat amylin-(8-37) and human CGRP-(8-37). All peptides have an intra-molecular disulfide bond between the 2^{nd} and 7^{th} Cys residues. The “NH₂” at the carboxy terminus represents amidation of the hormones.

FIG. 2A shows effects of amylin, CGRP, norepinephrine and insulin on muscle free fatty acid content. Soleus muscles were incubated for 1 h in KHB buffer (control), 100 nM amylin or CGRP-1, 4.4 μM norepinephrine or 23.7 nM insulin. Values are means±S.E.M. (n=8 for each group). *Significant difference compared to control (P<0.05). **Significant difference compared to control (P<0.01).

FIG. 2B shows effects of amylin, CGRP and norepinephrine on muscle glycerol content. Soleus muscles were incubated for 1 h in KHB buffer (control), 100 nM amylin or CGRP-1 or 4.4 μM norepinephrine. Values are means±S.E.M. (n=8 for each group). *Significant difference compared to control (P<0.05). **Significant difference compared to control (P<0.01).

FIG. 2C shows experiments in which amylin, CGRP and norepinephrine have no effects on muscle triglyceride. Soleus muscles were incubated for 1 h in KHB buffer (control), 100 nM amylin or CGRP-1 or 4.4 μM norepinephrine. Values are means±S.E.M. (n=8 for each group).

FIG. 3A shows effects of amylin-(8-37), CGRP-(8-37) and insulin on amylin's ability to increase intramuscular free fatty acids. Soleus muscles were incubated for 1 h KHB buffer (control), 100 nM amylin, 100 nM amylin with 10 μM amylin-(8-37) (A/A) or human CGRP-(8-37) (A/C), 10 μM amylin-(8-37) (A-8-37) or 100 nM amylin+23.7 nM insulin (A+I). Values are means±S.E.M. (n=7 at each point). **Significant difference compared to control (P<0.01).

FIG. 3B shows effects of amylin-(8-37), CGRP-(8-37) and insulin on the ability of CGRP-1 to increase intramuscular free fatty acids. Soleus muscles were incubated for 1 h in KHB buffer (control), 100 nM CGRP, 100 nM CGRP with 10 μM human CGRP-(8-37) (C/C) or amylin-(8-37) (C/A), 10 pM human CGRP-(8-37) (C-8-37) or 100 nM CGRP+23.7 nM insulin (C+I). Values are means±S.E.M. (n=7 at each point). **Significant difference compared to control (P<0.01).

FIG. 3C shows effects of amylin-(8-37) and CGRP-(8-37) on the ability of CGRP-1 to increase intramuscular free fatty acids. Soleus muscles were incubated for 1 h in KHB (control), 1 pM CGRP, 1 pM CGRP+100 pM CGRP-(8-37) or 1 pM CGRP+100 pM amylin-(8-37). Values are means±S.E.M. (n=7 at each point). **Significant difference compared to control (P<0.01).

FIGS. 4A and 4B show dose dependent effects of CGRP-1 to stimulate free fatty acid content in incubated rat soleus muscle. FIG. 4C shows the effects of CGRP-2 to stimulate free fatty acid content in incubated rat soleus muscle. Soleus muscle were incubated for 1 h in KHB (control) or a range of CGRP concentrations. Values are means±S.E.M. (n=7 at each point).

FIG. 5A shows dose dependent effects of amylin to stimulate free fatty acid content in incubated rat soleus muscle. Soleus muscles were incubated for 1 h in KHB (control) or a range of amylin concentrations. Values are means±S.E.M. (n=7 at each point).

FIG. 5B shows dose dependent effects of amylin to stimulate free fatty acid content in the presence of 100 pM CGRP. Soleus muscles were incubated for 1 h in KHB (control) or a range of amylin concentrations+100 pM CGRP. Values are means±S.E.M. (n=7 at each point).

FIGS. 6A, 6B and 6C show effects of amylin, CGRP and norepinephrine on the soleus muscle triglyceride content of rats fed high fat diets. Rats were fed a diet consisting 40% lard (6A), corn oil (6B) or olive oil (6C). Soleus muscles were incubated for 1 h in KHB (control), 100 nM amylin or CGRP-1 or norepinephrine 4.4 μM. Values are means±S.E.M. (n=7 at each point). *Significant difference compared to control (P<0.05). **Significant difference compared to control (P<0.01).

FIGS. 7A, 7B and 7C show effects of amylin, CGRP and norepinephrine on the soleus muscle free fatty acid content of rats fed high fat diets. Rats were fed a diet consisting 40% lard (7A), corn oil (7B) or olive oil (7C). Soleus muscles were incubated for 1 h in KHB (control), 100 nM amylin or CGRP-1 or norepinephrine 4.4 μM. Values are means±S.E.M. (n=7 at each point). *Significant difference compared to control (P<0.05). **Significant difference compared to control (P<0.01).

FIG. 8 shows total and non-specific binding of [³H]rat amylin or [³H]salmon calcitonin (sCT) to cultured L6 myoblasts that were transiently transfected with vectors containing inserts as described. Myoblasts were transfected as follows: A, vector alone; B, vector containing murine ramp 1; C, vector containing the insert-negative isoform of the rat calcitonin receptor 1, C1ᵢₙₛ₋; and D, co-transfection with vectors containing murine ramp 1 and murine C₁ᵢₙₛ₋, respectively. Binding was performed with concentrations of 10 nM of each radioligand in the absence (total (T)) or presence (non-specific (NS)) of 1 μM concentrations of corresponding unlabelled peptides, as shown. Specific binding may be obtained by subtraction of non-specific from the corresponding total binding in each case. Each bar represents the mean (±S.E.M.) of binding derived from three independent myoblast transfections from an experiment repeated at least twice. *, p<0.05; ***, p<0.001; significance of differences between total and corresponding non-specific binding was determined using unpaired Student's t-tests.

FIG. 9 shows displacement of bound [³H]rat amylin from L6 myoblasts co-transfected with vectors containing murine ramp 1 and the insert-negative rat calcitonin receptor isoform 1 (C1ᵢₙₛ₋). Myoblasts co-transfected with murine ramp 1 and C1ᵢₙₛ₋ were incubated with [³H]amylin (20 nM) in the presence of indicated concentrations of unlabelled ligands, for 3 h at 21° C. Cells were then washed and bound radioactivity determined by liquid scintillation counting. A, displacement of [³H]amylin by rat CGRP-1 (▴); rat amylin (∘); salmon calcitonin (★); rat calcitonin (8); or human adrenomedullin (:)B, displacement of [³H]amylin by ^{8,37}CGRP (Δ); or ^{8,37}amylin (6). Each point represents the mean±SEM of three independent myoblast transfections from an experiment repeated at least twice.

FIG. 10 shows effects of the truncated peptide antagonists ^{8,37}rat CGRP-1 and ^{8,37}rat amylin on cAMP concentrations in L6 myoblasts transiently co-transfected with murine receptor activity modifying protein 1, ramp 1, and the insert-negative murine calcitonin receptor isoform 1, C1ᵢₙₛ₋, and incubated with rat CGRP-1 or rat amylin. Transfected myoblasts were incubated with indicated concentrations of A, CGRP or B, amylin for 15 min. A: (▴) CGRP; (Δ)CGRP+^{8,37}CGRP (5 μM). B. (∘) amylin; (6) amylin+^{8,37}amylin (5 μM). Cells were washed and muscle cAMP content determined. Each point represent the mean (±SEM) of three independent transfections from an experiment performed at least twice.

FIG. 11 shows specific binding, expressed as a percentage of total binding, of A, [³H]rat CGRP-1, CGRP B,; [³H]rat amylin, amylin; and C, [³H]-sCT, to Cos-7 cells transiently transfected with vector constructs as indicated. Specific binding was derived from differences between total binding of the respective radioligands (20 nM in each case) in the absence or presence of corresponding unlabeled ligands (1 μM). Each point represent the mean±SEM of three independent transfections from an experiment performed at least twice. **, p<0.01 for binding compared with that to cells transfected with vector alone.

FIG. 12 shows Northern blots illustrating expression of RNA corresponding to murine receptor activity modifying protein 1, ramp 1, or murine ramp 3, in Cos-7 cells transiently transfected with stated vector constructs. (Upper panel): Northern blots of RNA extracted from cos-7 cells that had been transfected with vector alone, vector; or vector containing inserts corresponding to murine ramp 1 or ramp 3, then probed with labeled cDNAs corresponding to ramp 1 (left panel) or ramp 3 (right panel). (Lower panels): total RNA from cos-7 cells employed for Northern analysis, illustrating 18S and 28S rRNA bands to illustrate equivalent loading in each gel lane.

FIG. 13 shows concentration-dependent effects of CGRP and amylin on cAMP in rat skeletal muscle. Soleus muscle strips were incubated in vitro with rat CGRP-1 (13A), or rat amylin (13B, 13C), at the concentrations shown. Incubations were performed in the absence or presence of human insulin (23.7 nM). Statistical significance was tested by one-way ANOVA, followed by post-hoc analysis using Dunnett's Multiple Comparisons Test, A, *p<0.05, **p<0.01, compared to control; and B, *p<0.05, **p<0.01 compared to control. cAMP content measured in soleus muscles treated with 1 μM isoproterenol was 3.5±0.2 pmol/mg (p<0.05) and 3.8±0.2 pmol/mg (p<0.01) compared to control for A and B, respectively.

FIG. 14 shows effects of peptide antagonists on amylin-mediated suppression of insulin-stimulated glucose transport in rat soleus muscle in vitro. Rat soleus muscle was isolated, stripped and incubated with amylin (10 nM) and insulin (23.7 nM), as well as with the indicated concentrations of A, ^{8,37}rat amylin or B, ^{8,37}rat CGRP-1, and glucose transport determined as uptake of 2-deoxy-D-glucose (2-DOG; nmol/g-dry wt/min). Points represent means (±S.E.M.) from n=4 independent experiments. Statistical significance of the effect was tested by one-way ANOVA, followed by post hoc analysis using Dunnett's Multiple Comparisons Test; *p<0.05, **p<0.01 compared with control values corresponding to [insulin (23.7 nM)+amylin (10 nM)] alone (no antagonist).

FIG. 15 shows the effect of CGRP and related peptides on basal and insulin-stimulated in vitro metabolism in rat skeletal muscle. Dose-response curves were derived for: (A, B), total glycogen content; and (C, D)), rates of incorporation of D[¹⁴C(U)]glucose into glycogen, in isolated rat soleus muscle strips incubated in the presence of indicated concentrations of rat amylin, amylin (0); rat CGRP-1, CGRP (▴); or salmon calcitonin, sCT (*). Incubations were performed in the absence (A, C), or presence (B, D), of maximally effective human insulin (23.7 nM). Each point represents the mean (±S.E.M.) from n=4-5 independent experiments (total glycogen) and from n=3-6 (D[¹⁴C(U)]glucose incorporation) and each determination was performed in triplicate, except that basal values were derived from n=15 or 12 independent experiments, respectively.

FIG. 16 shows insulin dose-repsonse curves in soleus muscle from rats fed a high fat diet. Insulin dose response curves were measured in soleus muscle from rats fed a normal diet (′) or a high fat diet (□) for 51 days. Insulin responses were measured through incorporation of D[¹⁴C(U)] glucose into muscle glycogen following incubation for 2 h with various concentrations of insulin. Muscle glycogen was then extracted and analysed for D[¹⁴C(U)] glucose content by liquid scintillation spectrometry. Dose-respone curves were fitted to a sigmoidal dose-response algorithm. Results are shown as means±SEM (n=12-18 for each point). Sum of squares analysis revealed significant differences between the two curves (P<10-5).

FIGS. 17A and 17B show the effect of CGRP-1 on muscle and triglyceride content in high-fat fed animals.

FIG. 18 shows the effect of receptor antagonists of CGRP on CGRP-1 effects on muscle lipid in high fat-fed rats The statistical significance of the results shown was on anlyzed by one way ANOVA and post-hoc analysis using Tukey's test. ***p<0.0001, *P<0.05 compared to basal; ###P<0.001 compared to 100 nM CGRP; ′″ P<0.0001 compared to 1 pM CGRP.

FIG. 19 shows the effects of CGRP-1 on cAMP content in soleus muscle from normal fed and high fat-fed animals.

FIG. 20 shows a drop in mean arterial pressure in Wistar rats infused with rat CGRP 1 (100 pmol/kg/min) or saline for 1 h. Mean arterial pressure was continuously measured using a solid-state blood pressure transducer, and monitored using a PowerLab/16s data acquisition module. Calibrated signals were displayed on screen and saved to disc as 2 s averages of each variable. * P<0.05.

FIG. 21 shows the effect on blood glucose levels of infusion with CGRP-1 or an agonist of CGRP-1 activity. Blood glucose levels were determined at 5-minute intervals using an Advantage meter and tail blood samples from animals infused for 90 minutes as indicated.

### DETAILED DESCRIPTION OF THE INVENTION

### I. Definitions

As used herein, “lipolysis” refers to enzymatic hydrolysis of lipids or fat storage compounds such as triglycerides, causing the release of free fatty acids. The terms “free fatty acids (FFA)” and “non-esterified fatty acids (NEFA)” are used interchangeably herein.

As used herein, “therapeutically effective amount” refers to a predetermined amount of an agent calculated to elicit the biological or medical response of a tissue, system, animal or human that is being sought by the researcher, veterinarian, physician or other clinician, e.g., an amount sufficient to stimulate lipolysis or reduce lipid levels and/or ameliorate a disease state or symptoms, or otherwise prevent, hinder, retard or reverse the progression of a disease or any other undesirable symptoms to achieve a desired therapeutic effect.

As used herein, “pharmaceutically acceptable carrier” or “pharmaceutically acceptable excipient” refers to a carrier that does not cause an adverse physical reaction upon administration and one in which a therapeutic agent is sufficiently soluble to deliver a therapeutically effective amount. Examples of excipients include buffered water, physiological saline, PBS, dextrose solution, Hank's solution and inert diluents, such as calcium carbonate, sodium carbonate, lactose, calcium phosphate or sodium phosphate.

As used herein, “mammal” has its usual meaning and includes primates (e.g., humans and nonhuman primates), experimental animals (e.g., rodents such as mice and rats), farm animals (such as cows, hogs, sheep, and horses), and domestic animals (such as dogs and cats).

As used herein, the terms “treatment” or “treating” of a condition and/or a disease in a mammal, means (i) preventing the condition or disease, that is, avoiding any clinical symptoms of the disease; (ii) inhibiting the condition or disease, that is, arresting the development or progression of clinical symptoms; and/or (iii) relieving the condition or disease, that is, causing the regression of clinical symptoms.

As used herein, “EC₅₀” has its normal meaning in the art and refers to a concentration of a compound that results in 50% of maximum enhancement of a specified biological effect, e.g., the concentration at which a biological effect mediated by binding of a ligand (e.g., CGRP) to a receptor (e.g., the high affinity CGRP receptor) is at one-half of its maximum value. For example, the EC₅₀ for stimulation of skeletal muscle lipolysis by CGRP and the high affinity CGRP receptor is the concentration of CGRP that results in 50% of maximum enhancement of skeletal muscle lipolysis over a baseline level. The ligand may or may not be naturally occurring.

As used herein, a receptor “agonist” is an agent (compound or composition) capable of promoting at least one of the biological responses normally associated with binding of a natural ligand of the receptor to the receptor. For example, an agonist of the high affinity CGRP receptor present in skeletal muscle cells is an agent (e.g., CGRP) that interacts with (e.g., binds) the high affinity receptor and increases lipolysis in the muscle cells in a dose-dependent manner. A binding interaction between an agonist and a CGRP receptor can be determined by the ability of the agonist to compete with CGRP (e.g., radiolabeled CGRP) for binding to the receptor and/or using other competition assays.

As used herein, a receptor is “stimulated” or, equivalently, is “activated” or, equivalently, is “agonized” by a compound or agent when binding of the compound or agent to the receptor (e.g., to the ligand binding site) results in a change in the metabolic state of the cell expressing the receptor. Usually, upon binding of the ligand (e.g., a natural ligand or other agonist) the receptor transduces a signal to the cell interior. When contacting a receptor with a particular compound results in a response by the cell characteristic of the response observed upon binding of a natural (i.e., naturally occurring endogenous) ligand, this is an indication that the compound is capable of stimulating or activating the receptor. Grammatical equivalents (e.g., activation, stimulation, etc.) have the corresponding meaning.

As used herein, “conservative substitution,” when describing a protein, refers to a change in the amino acid composition of the protein in which residues are replaced with structurally similar substitutes that do not substantially alter the protein's activity. Thus, “conservatively substituted variants” of a particular amino acid sequence refers to amino acid substitutions of those amino acids that are not critical for protein activity, or substitution of amino acids with other amino acids having similar properties (e.g., acidic, basic, positively or negatively charged, polar or non-polar, etc.) such that the substitutions of even critical amino acids does not substantially alter activity. Conservative substitution Tables providing functionally similar amino acids are well known in the art. The following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (1), Leucine (L), Methionine (M), Valine (V); and 6) Phenylalanine (F), Tyrosine (Y), Tryptophan (W) (see also, Creighton (1984) Proteins, W. H. Freeman and Company). One of skill in the art will appreciate that the above-identified substitutions are not the only possible conservative substitutions. For example, one may regard all charged amino acids as conservative substitutions for each other whether they are positive or negative. In addition, individual substitutions, deletions or additions which alter, add or delete a single amino acid or a small percentage of amino acids in an encoded sequence can also be “conservatively substituted variants.”

The term “peptide” refers to a polypeptide fewer than 50 residues in length. The terms “protein” and “polypeptide” encompass both “peptides” and longer amino acid polymers.

The term “contacting,” has its normal meaning of bringing in proximity. Methods of contacting an compound and a receptor expressed by a cell or tissue include, without limitation, incubating the cell or tissue ex vivo (e.g., in vitro) together with the compound under conditions under which the compound can bind the receptor, and administration of the compound to an animal such that the compound is delivered to the cell or tissue by circulatory or other system of the animal. In some cases, the agent administered is converted to the agonist by metabolic activity of the animal or tissue (e.g., a metabolite of the administered agent is an agonist).

As used herein, the terms “peptide mimetics” or “peptidomimetics” (Fauchere, 1986, Adv. Drug Res. 15:29; Veber and Freidinger, 1985, TINS p.392; and Evans et al., 1987, J. Med. Chem 30:1229) have their usual meaning in the art refer to peptide analogs used in the pharmaceutical industry as non-peptide drugs with properties analogous to those of the template peptide. Peptidomiimetics are usually developed with the aid of computerized molecular modeling. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biological or pharmacological activity), such as naturally-occurring CGRP polypeptide, but have one or more peptide linkages optionally replaced by a linkage selected from the group consisting of: —CH₂NH—, —CH₂S—, —CH₂—CH₂—, —CH.dbd.CH—(cis and trans), —COCH₂—, —CH(OH)CH₂—, and —CH₂ SO—, by methods known in the art and further described in the following references: Spatola, A. F. in “Chemistry and Biochemistry of Amino Acids, Peptides, and Proteins,” B. Weinstein, eds., Marcel Dekker, New York, p. 267 (1983); Spatola, 1983, Vega Data 1:3, “Peptide Backbone Modifications”; Morley, 1980, Trends Pharm Sci pp. 463-468; Hudson, D. et al., 1979, Int J Pept Prot Res 4:177-185 (—CH₂NH—, CH₂CH₂—); Spatola et al., 1986, Life Sci 38:1243-1249 (—CH₂—S); Hann, M. M., J Chem Soc Perkin Trans I (1982) 307-314 (—CH—CH—, cis and trans); Almquist, R. G. et al., J Med Chem (1980) 23:1392-1398 (—OCH₂—); Jennings-White et al., Tetrahedron Lett (1982) 23:2533 (—OCH₂—); Szelke et al., European App. EP 45665 (1982) CA: 97:39405 (1982) (—CH(OH)CH₂—); Holladay, M. W. et al., Tetrahedron Lett (1983) 24:4401-4404 (—C(OH)CH₂—); and Hruby, V. J., Life Sci (1982) 31:189-199 (—CH₂—S—). Such peptide mimetics may have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others. Peptidomimetic compounds can also contain biological equivalents of amino acids such as the sulfonic and boronic acid analogs of amino acids.

As used herein, the term “normalize,” when used in the context of lipid content in skeletal muscle, refers to a reduction in lipid content in muscle of individuals with abnormally high levels of muscle triglyceride, e.g., individuals who would benefit from a reduction in muscle triglyceride.

As used herein, the term “minimal,” when referring to the effect of a compound on a metabolic response (e.g., increase in blood glucose or lactate levels) of an animal, tissue, or cell refers to an effect of a compound that mediates an increase in lipolysis by stimulating the high affinity receptor, but does not significantly increase the metabolic response due to stimulation of the amylin metabolic receptor. For example, in one embodiment, the terms “minimally stimulates vasodilatation” “minimally stimulates side-effects” “or minimally increases blood lactate” and the like, can refer to an agent or amount of agent for which the ratio: [increase in lipolysis]/[increase in blood glucose/elevation of blood lactate/increase in vasodilatation/increase in side-effects, etc.] is higher than the ratio resulting from stimulation by a metabolic amylin receptor saturating amount of CGRP-1 (e.g., 200 nM). Usually the ratio is at least about 2-fold higher, often at least about 4-fold higher, sometimes at least about 10-fold higher). Also encompassed by these terms are agents or doses for which no significant increase in vasodilatation and/or elevation of blood lactate and/or other side-effects is detected (i.e. where the denominator is close to zero).

### II. Description

### A. Introduction

Calcitonin gene-related peptide (CGRP) is a 37 amino acid member of the calcitonin family of peptide hormones. CGRP is present in efferent, acetylcholine containing neurons innervating motor end plates of skeletal muscles where it is released into the synaptic space after nerve stimulation. CGRP is found in humans in at least two predominant forms, called CGRP-1 and CGRP-2 (Table 1).

TABLE 1

AMINO ACID SEQUENCES OF HUMAN AND RAT
CGRP-1 AND CGRP-2

| | |
|---|---|
| hCGRP-1 | Ala Cys Asp Thr Ala Thr Cys Val Thr His |
| | Arg Leu Ala Gly Leu Leu Ser Arg Ser Gly |
| | Gly Val Val Lys Asn Asn Phe Val Pro Thr |
| | Asn Val Gly Ser Lys Ala Phe |
| hCGRP-2 | Ala Cys Asn Thr Ala Thr Cys Val Thr His |
| | Arg Leu Ala Gly Leu Leu Ser Arg Ser Gly |
| | Gly Met Val Lys Ser Asn Phe Val Pro Thr |
| | Asn Val Gly Ser Lys Ala Phe |
| rCGRP-1 | Ser Cys Asn Thr Ala Thr Cys Val Thr His |
| | Arg Leu Ala Gly Leu Leu Ser Arg Ser Gly |
| | Gly Val Val Lys Asp Asn Phe Val Pro Thr |
| | Asn Val Gly Ser Glu Ala Phe |
| rCGRP-2 | Ser Cys Asn Thr Ala Thr Cys Val Thr His |
| | Arg Leu Ala Gly Leu Leu Ser Arg Ser Gly |
| | Gly Val Val Lys Asp Asn Phe Val Pro Thr |
| | Asn Val Gly Ser Lys Ala Phe |

CGRP shares approximately 50% sequence similarity to the protein amylin (Cooper et al., 1987, Proc Natl Acad Sci USA 84:8628-32; Cooper et al., 1994, Endocr Rev. 15:163-201) and the two proteins have overlapping, but different, sets of biological activities (see, e.g., Muff et al., 1995, Eur. J Endocrinol. 133:17-20). Among its activities, CGRP has significant cardiovascular effects, including vasodilatation and positive chronotropic and inotropic effects (Nuki et al., 1993, Biochem Biophys Res Commun 196:245-51; Muff et al., supra; Cooper et al., 1994, Endocr Rev. 15:163-201; Gardiner, 1991, Diabetes 40:948-951; and Takenaga, 1999, Euro. J. Pharma. 367:239-245). In addition, when administered at high concentration, CGRP is reported to lead to insulin resistance (see, e.g., Leighton and Cooper, 1988, Nature 335:632-35). It has been proposed that insulin resistance should be treated by inhibiting the release or activity of CGRP (see, e.g., U.S. Pat. No. 6,004,961; U.S. Pat. No. 5,641,744). In the rat, elevated plasma amylin and CGRP levels are associated with insulin resistance, and in humans, with obesity and type 2 diabetes. In muscle, amylin and CGRP have been reported to inhibit glucose uptake by inhibition of glycogen synthesis and to promote glycogenolysis, opposing the actions of insulin as non-competitive, functional antagonists. The elevated circulating lactate from glycogenolysis serves as a substrate for liver gluconeogenesis, causing excessive hepatic glucose production.

The present invention is related, in part, to the discovery that CGRP-1 stimulates lipolysis (i.e., cause the breakdown of triacylglycerol [triglycerides]) in skeletal muscle and liver, and rat CGRP-2 was demonstrated to stimulate lipolysis in muscle. This breakdown can be detected as an increase of free fatty acids. As shown in the examples, infra, dose-response assays in which skeletal muscle was exposed to various amounts of rat CGRP-1 show a biphasic increase in muscle free fatty acid levels, with a first phase observed at a picomolar concentration of CGRP-1 and a second phase observed at a nanomolar concentration of CGRP-1 or 2. Without intending to be bound by a particular mechanism, the data indicate that potent lipolysis effects of CGRP-1 on muscle lipid are effected via a high affinity CGRP receptor (EC₅₀ about 2.6×10⁻¹³ M [e.g., range 10⁻¹¹ M to 10⁻¹³ M]). Thus, a cell that expresses the high affinity receptor is characterized by responding to picomolar concentrations of CGRP-1. This concentration is much lower than those that elicit insulin resistance and inhibition of glycogen synthesis in isolated muscle preparations (Leighton et al., 1988, Nature 335:632-635; Leighton et al., 1989, FEBS Lett 249:357-361). A second phase of lipolysis occurs at higher concentrations of CGRP-1 or 2 (EC₅₀ about 4.5×10⁻⁸ M [e.g., range 10⁻⁹ M to 10⁻⁸ M]) and is mediated by the metabolic amylin receptor, i.e., a single receptor that can transduce signaling by both CGRP and amylin in skeletal muscle, where it can act as a receptor for either peptide (that is, a CGRP/amylin receptor).

As described in greater detail hereinbelow, an agonist of the high affinity CGRP receptor is characterized by a dose-dependent stimulation of lipolysis in skeletal muscle, in which stimulation is mediated by the receptor with which CGRP-1 interacts with a picomolar EC₅₀. An agonist of the metabolic amylin receptor is characterized by stimulation of lipolysis in skeletal muscle mediated by the receptor through which CGRP-1 or 2 interacts with a nanomolar EC₅₀, and with which amylin elicits lipolytic effects.

A receptor with the characteristics of the metabolic amylin receptor is constituted by co-transfection of the calcitonin receptor (insert-negative form) [C1ᵢₙₛ₋] with ramp 1 into either L6 myoblasts or cos-7 cells (see Examples, infra). This receptor constitutes strong specific binding of CGRP, amylin and sCT, and transduction of signals by both CGRP and amylin. High affinity binding sites for CGRP-1 have been described in muscle and liver cells. Galeazza et al., 1991, Peptides 12:585-91 described two CGRP-1 binding sites in rat skeletal muscle membrane preparations, with derived Kd values of approximately 37 pM and 6 nM. Galeazza et al. also described two specific binding sites for CGRP in rat liver membranes, with derived Kd values of approximately 44 pM and 27 nM. Binding of radiolabeled CGRP-1 to membrane fractions of liver nonparenchymal cells (e.g., endothelial, lipid storage, and smooth muscle cells) but not in parenchymal liver cells was reported by Stephens et al., 1991, Diabetes, 40:395-400. Also see Poyner et al., 2002, Pharmacol. Rev. 54:233-46.

The discovery that lipolysis can be mediated via the high affinity CGRP receptor, and other discoveries detailed herein, provide new methods and reagents for stimulation of lipolysis and reduction of accumulation of lipid in cells and tissues. In particular, the invention provides methods and reagents to effect reduction of lipids in skeletal muscle and liver, and for treatment of diseases characterized by accumulation of lipid in cells and tissues. Accumulation of lipid in skeletal muscle is a key metabolic abnormality underlying the development of insulin resistance. For example, in obesity-related insulin resistance, the metabolic capacity of skeletal muscle appears to be organized towards fat esterification rather than oxidation, and dietary-induced weight loss does not correct this disposition (Simoneau et al., 1999, FASEB J. 13:2051-60). There is also evidence that normalization of muscle lipid is accompanied by an increase in insulin sensitivity (see, e.g., McGarry et al., 1992, Science 258:766-70; Ellis et al., 2000, Am J Physiol Endocrinol Metab. 279:E554-60; Oakes et al., 1997, Diabetes 46:1768-74; Furler et al., 1997, Metabolism 46:1101-6; Kraegen et al., 1991, Diabetes 40:1397-403; Dobbins et al., 2001, Diabetes 50:123-30; Unger et al., 2000, Int J Obes Relat Metab Disord. 24 Suppl 4:S28-32; Manco et al., 2000, Metabolism 49:220-4; Kelley et al., 1999, Physiol. 277(6 Pt 1):E1130-41).

Diseases and conditions characterized by accumulation of lipid in skeletal muscle include insulin resistance and associated conditions, such as those of Syndrome X (e.g., type-2 diabetes mellitus, hypertension, obesity, dyslipidaemia, atherosclerosis, and thrombosis) and poly cystic ovary syndrome (PCOS). Diseases and conditions characterized by accumulation of lipid in liver include fatty liver (hepatic steatosis). Hepatic steatosis can arise in patients with solvent injury (e.g., from carbon tetrachloride) or chronic excessive alcohol consumption, obese patients and as a result of administration of drugs such as glucocorticoids and synthetic estrogens. In one aspect, the invention provides methods and reagents for treatment of these and other conditions.

The invention also provides methods for identification and development of phamaceutical compositions useful for stimulating lipolysis in muscle and other tissues.

Further, it has been observed that knockout of the CGRP-1 gene in mice did not affect cardiovascular regulation (Lu et al., 1999, Mol. Cell. Neuroscience 14:99-120). Thus, in one aspect, the present invention provides methods for treating insulin resistance using an agonist of the high affinity CGRP receptor normally bound by CGRP-1, while mimizing or avoiding the adverse side effects (e.g., vasodilatation; acute cardiovascular effects) that normally accompany administration of high doses CGRP. Still further, it is believed that the beneficial effects of lipolysis can be achieved, while minimizing the increase in circulating lactate (e.g., resulting from glycogenolysis) and elevated blood glucose (e.g., resulting from lactate-stimulated hepatic glucose production).

### B. Reduction of Tissue Lipid Content, and Treatment of Subjects who will Benefit From a Reduction in Lipid Levels (e.g., in Skeletal Muscle and/or Liver)

In one aspect, the present invention provides a method of reducing lipid content in a tissue or cell (e.g., skeletal muscle or liver) by contacting the tissue or cell with an agonist of the high affinity CGRP receptor or the metabolic amylin receptor. In a preferred aspect, the present invention provides a method of reducing lipid content in skeletal muscle or liver tissue or a skeletal muscle or liver cell, by contacting the tissue or cell with an agonist of the high affinity CGRP receptor. As noted above, such reduction is accompanied by a variety of beneficial effects. In one embodiment, a tissue or cell is contacted with an agonist by administration of the agonist to an animal, typically a mammal.

The invention provides a method for treating a mammal in need of a reduction in lipid content (for example, of skeletal muscle or liver) to ameliorate symptoms of, or reduce likelihood of developing, conditions associated with muscle fat accumulation, particularly including insulin resistance and “syndrome X” complex (e.g., type-2 diabetes mellitus, hypertension, obesity, dyslipidaemia, atherosclerosis, and thrombosis), polycystic ovary syndrome, and hepatic steatosis, by administering an agonist of the high affinity CGRP receptor. In an embodiment the agonist is human CGRP, e.g., human CGRP-1. In an embodiment, the agonist of the high affinity CGRP receptor does not substantially stimulate the metabolic amylin receptor. In embodiments of this and other methods of the invention, the mammal may be a human, a primate, a nonhuman primate, or a nonhuman animal.

In a related aspect, the invention provides a method of treating a mammal, such as a human, in need of a reduction in lipid content (for example, of skeletal muscle or liver) to ameliorate symptoms of, or reduce likelihood of developing, conditions associated with muscle fat accumulation, by (1) identifying an animal with a condition associated with fat accumulation (e.g., insulin resistance, type-2 diabetes mellitus, hypertension, obesity, dyslipidaemia, atherosclerosis, thrombosis, polycystic ovary syndrome, or hepatic steatosis) (2) administering an agonist of the high affinity CGRP receptor, and (3) monitoring lipolysis in the animal. In an embodiment, the condition is insulin resistance. In an embodiment, the condition is type-2 diabetes mellitus. In an embodiment, the mammal is insulin resistant but does not have type II diabetes. In an embodiment, the mammal is not treated with insulin. Identification of a mammal as suffering from an aforementioned condition is readily accomplished using art known diagnostic methods (including methods described hereinbelow) but can be accomplished using any identification method known or developed in the future (e.g., gene expression profiling). An exemplary agonist of of the high affinity CGRP receptor is human CGRP-1 and biologically functional variants of human CGRP-1. In an embodiment, administration of the agonist does not substantially stimulate the metabolic amylin receptor.

In an aspect, the invention provides the use of an agonist of the high affinity CGRP receptor for the manufacture or formulation of a medicament for reduction of lipid levels in a subject who would benefit from such a reduction. In an aspect, the invention provides the use of an agonist of the high affinity CGRP receptor for the manufacture or formulation of a medicament for treatment of a subject with Syndrome X syndrome, insulin resistance, type-2 diabetes mellitus, hypertension, obesity, dyslipidaemia, atherosclerosis, and thrombosis.

In a related aspect, the invention provides pharmaceutical compositions in unit dosage form which contain an agonist of the high affinity receptor as described herein. For example, in one embodiment, the unit dosage results, when administered to a mammal such as a human, in a blood or plasma level of agonist that is below the EC₅₀ of the active agent for the metabolic amylin receptor. Usually the blood or plasma level of agonist of the agent is present between about 10⁻¹⁵ M and about 10⁻¹⁰ M). For CGRP-1, the plasma level may be below 300 pM (see Examples, infra).

Suitable receptor agonists (e.g., of the high affinity CGRP receptor) can be any of a variety of agents, as discussed in detail infra, including a naturally occurring CGRP polypeptide (e.g., human CGRP-1 polypeptide), a CGRP polypeptide variant (e.g., a biologically functional variant), or a non-polypeptide agent.

Administration of the CGRP receptor agonist to a subject (i.e., in vivo contacting) or ex vivo contacting a tissue or cell with the agonist, can result in a detectable reduction in lipid content and concomitant increase in free fatty acids. It is believed that lipid content is reduced as a consequence of stimulation of lipolysis in cells, e.g., skeletal muscle cells. See, e.g., Example 17, infra. However, applicants do not intend to be bound by any particular mechanism. Thus, while phrase “stimulation of lipolysis” is sometimes used herein for convenience, it is intended to encompass a reduction of lipid levels and increase in free fatty acids in a tissue, without regard to the mechanism of the reduction and increase. CGRP-receptor agonist stimulation of lipolysis can be detected as a change in amount or composition of free fatty acids in a tissue (e.g., skeletal muscle or liver, or blood or blood plasma). Usually, the level of stimulation of lipolysis is an increase in tissue or blood free fatty acid levels of at least about 2-fold, often at least about 4-fold, and sometimes at least about 6-fold or more over baselines (levels in the absence of the agonist). Methods for detecting a change in free fatty acid levels are well known. For example, as described in Example 2, infra, gas chromatographic analysis of muscle tissue (e.g., obtained from biopsy or tissue culture) can be conducted. Other methods include HPLC procedures for preparative scale separations of particular fatty acids for structural or metabolic studies. This technique is useful for volatile components, such as short chain fatty acids, for studying isotopically labeled fatty acids, or for separation of positional and conformational isomers. Derivatized fatty acids are usually monitored with UV spectrophotometer or by fluorimetry (Rao et al. 1995, J Chromatogr Sci 33:9-21). Plasma concentrations of non-esterified free fatty acids can be determined by an acyl CoA oxidase-based colorimetric assay method (Wako Pure Chemical Industries, Osaka, Japan). Plasma triglcerides can be measured using a colorimetric assayed (Triglyceride Procedure 336, Sigma Diagnostics). Tissue long chain CoA can be measured by solvent extraction of long chain CoA from tissues, phase separation, and purification by reverse phase high performance liquid chromatography.

In an aspect, the invention provides the use of an agonist of the high affinity CGRP receptor for the manufacture or formulation of a medicament for reduction of lipid levels in a subject who would benefit from such a reduction. In an aspect, the invention provides the use of an agonist of the hight affinity CGRP receptor for the manufacture or formulation of a medicament for treatment of a subject with Syndrome X syndrome, insulin resistance, type-2 diabetes mellitus, hypertension, obesity, dyslipidaemia, atherosclerosis, and thrombosis.

Although agents that agonize the high affinity receptor are of particular interest, in a different aspect, the invention provides a method of treating a mammal in need of a reduction in lipid content comprises administering an agent that is an agonist of the metabolic amylin receptor, but not the high affinity receptor, to stimulate lipolysis in a mammal, methods of treating mammals in need of treatment for lipolysis reduction by administering such an agonist of the metabolic receptor, and pharmaceutical compositions comprising such an agonist to achieve the result. In one embodiment the agonist is rat CGRP-2. In one embodiment the agonist is a receptor stimulating varient of CGRP. In an embodiment, the agonist is not amylin. In an alternative embodiment, the agonist is amylin. Methods and modes of administration for such an agonist are analogous to those described herein for agonists of the high affinity receptor, with changes in doses, etc, that will be apparent to the skilled reader.

### C. Agonists of the High Affinity CGRP Receptor

Agonists of the high affinity CGRP receptor used in the practice of the invention include a variety of types of compounds, including peptides, peptide mimetics and nonpeptide compounds. Suitable agonists are described below and/or can be identified using methods described herein (and/or by methods that will be apparent to one of ordinary skill guided by this disclosure). Agonists of the high affinity CGRP receptor stimulate lipolysis in skeletal muscle. Preferred agonists and/or dosages result in preferential stimulation of the high affinity CGRP receptor compared to the metabolic amylin receptor.

Preferential Stimulation

As noted herein, certain tissues (e.g., skeletal muscle) express both the high affinity receptor and the metabolic amylin receptor, and certain compounds, such as rat CGRP-1, are agonists of both the high affinity CGRP receptor and the metabolic amylin receptor. As noted above, lipolysis can be mediated by both receptors. However, as disclosed herein, it will often be desirable to preferentially stimulate the high affinity “Preferential stimulation” has the normal meaning of the term and can be described (or detected) in a variety of ways. “Preferential stimulation” occurs when the agonist of the high affinity receptor has an EC₅₀ for the high affinity receptor that is lower than the EC₅₀ for the metabolic receptor, and the dose or concentration of the agonist is below the EC₅₀ for the metabolic amylin receptor but at or above the EC₅₀ for the high affinity receptor. In one embodiment, “preferential stimulation” is accomplished when the high affinity receptor agonist does not cause any detectable stimulation of the metabolic amylin receptor (e.g., at any dose, or, alternatively, at any dose less than 10 microM, or any dose less than 10 mM).

Thus, in one embodiment of the invention, the agonist of the high affinity CGRP receptor is administered in an amount that results in preferential stimulation of the high affinity CGRP receptor compared to the metabolic amylin receptor. Such preferential stimulation can be achieved, e.g., by adjusting the dose or formulation of an agonist, or due to the properties of the agonist.

In one embodiment, preferential stimulation of the high affinity CGRP receptor compared to the metabolic amylin receptor is accomplished by contacting the cell with an agent that causes preferential stimulation of the high affinity receptor. In one embodiment, the agent is an agonist of the high affinity CGRP receptor but does not substantially agonize the metabolic amylin receptor. In an embodiment, the agent has an EC₅₀ for effecting lipolysis stimulation via the metabolic amylin receptor significantly greater than the EC₅₀ of the agent for effecting lipolysis stimulation via the high affinity CGRP receptor. In an embodiment, contacting skeletal muscle with the agent results in greater production of FFA (e.g., as measured by appearance of FFA in tissue or plasma) due to the lipolysis-stimulating effects of the high affinity receptor than due to the lipolysis-stimulating effects of the metabolic amylin receptor. It will be appreciated that one way to determine whether an effect of an agent is a result of stimulation of the high affinity CGRP receptor is by comparing the effect of the agent with the effects of amylin (which has a similar dose response curve as CGRP) which is known to act via the metabolic receptor.

In certain embodiments, the agent with an EC₅₀ for effecting lipolysis stimulation via the metabolic amylin receptor significantly greater than the EC₅₀ of the agent for effecting lipolysis stimulation via the high affinity CGRP receptor has an EC₅₀ for the high affinity CGRP receptor at least 10-fold less than for the metabolic amylin receptor, and more often at least about 100-fold less, at least about 500-fold less, at least about 1000-fold less, at least about 5000-fold or at least about 10,000-fold less. Most often, however, the EC₅₀ for the high affinity receptor is at least about 10⁵-fold less, 10⁶-fold less, 10⁷-fold less, or 10⁸-fold less. Described as a ratio of EC₅₀ for the low affinity receptor (“EC_{50-amylin-R}”) to the EC₅₀ of the high affinity receptor (“EC_{50-CGRP-R}”), i.e., a EC_{50-amylin-R}/EC_{50-CGRP-R} ratio, the ratio for an agent with a significantly greater EC₅₀ for stimulation via the high affinity receptor is usually at least about 10, often at least about more than about 10², more than 5×10², more than about 10³, more than 5×10³, more than about 10⁴, more than about 10⁵, more than about 10⁶, more than about 10⁷ or more than about 10⁸. Methods for determining the EC₅₀ of a compound are known generally, and are described hereinbelow.

In some embodiments, the agonist of the high affinity CGRP receptor does not have any significant or any detectable stimulatory activity for the metabolic amylin receptor when administered or contacted with a cell or tissue. One measure of significant stimulatory activity for the metabolic amylin receptor is a significant increase in blood lactate and blood glucose resulting from infusion of the agonist into an animal (e.g,. rat) relative to a control animal infused with saline or other non active composition. Significance is determined by routine methods such as the mean and standard error inherent in the measurements. Significance between control versus treated means can be tested statistically using standard analyses such as Student's t-test. Usually, assays are conducted using an n of >1, e.g., at least 7 for greater statistical power. Assays for increased lactate production and antagonism of insulin-stimulated glucose uptake in skeletal muscle via the metabolic amylin receptor are well known (see, e.g., Cooper, 1994, Endocr Rev. 15:163-201; Young et al., 1993, FEBS Lett. 334 (3) 317-321; and Young et al., “Amylin Activity Assays” U.S. Pat. No. 6,048,514).

Other assays can be carried out using human cells or tissues, cells or tissues from other mammals (e.g., mice or rats), cell lines, and recombinant cells. Assays include cell-based assays; ex vivo assays (e.g., in isolated rat soleus muscle), and whole animal studies. For example, cell-based assays can be carried out using the cell-based recombinant receptor described in the Examples, infra.

In one assay, isolated rat soleus muscle is used. Rat soleus muscle contains both the high affinity CGRP receptor and the metabolic amylin receptor and isolated rat soleus muscle is particularly useful to distinguish differential effects on the different receptors. The soleus muscles can be derived from animals that have been fed a high fat or normal diet and dose-response curves for a compound prepared in which (i) assays reflective of processes that can occur through both the high affinity CGRP receptor and the metabolic amylin receptor (e.g., measurement of muscle free fatty acid and triglyceride content), and (ii) reflective of processes occurring through the metabolic amylin receptor (e.g., measurement of muscle glycogen content and [¹⁴C]-glucose incorporation into glycogen in the presence of maximally-stimulating insulin) are made. It has been shown previously that both amylin and CGRP have similar effects on glycogen metabolism, in particular, the nontcompetitive antagonism of insulin-stimulated glucose incorporation into glycogen (for exemplary assays, see, e.g., Muff et al., supra; Cooper et al., 1994, Endocr Rev. 15:163-201; Gardiner, 1991, Diabetes 40:948-951; Takenaga, 1999, Euro. J. Pharma. 367:239-245, Leighton et al., 1988, Nature 335:632-5; Cooper et al., 1988, Proc.Nat.Acad.Sci U.S.A. 85:7763-6). An agent, dose or formulation that elicits effects indicating stimulation of the high affinity CGRP receptor but not effects indicative of stimulation specific to the metabolic amylin receptor are considered agonist of the high affinity CGRP receptor that do not substantially agonize the metabolic amylin receptor. In one embodiment, the effects on the high affinity and metabolic receptors are distinguished by selective antagonists of the high affinity and/or metabolic receptor. An agent or treatment that does not cause a significant change in an activity of the metabolic amylin receptor (e.g., an increase in receptor-mediated lipolysis) compared to control tissues, cells or animals (e.g., not exposed to the agonist) is considered to not substantially stimulate activity of the metabolic amylin receptor. Additional assays to determine preferential stimulation of the high affinity receptor are provided herein (e.g., see Examples).

As noted above, in one embodiment of the invention, the agonist of the high affinity receptor is administered to a mammal (or contacted with an isolated tissue) at a dose sufficient to stimulate the skeletal muscle high affinity CGRP receptor in the host without substantial stimulation of the low affinity skeletal muscle metabolic amylin receptor. Thus, the effect of an agent that stimulates both the high affinity receptor and the metabolic amylin receptor at high concentration, can be modulated to maximize relative to any stimulation of the metabolic amylin receptor by adjusting the dose administered (for example, adjusting the formulation such that only a measured amount of active compound is released and available to interact with the receptor). As used herein, “dose administered” in intended to include ex vivo contacting of tissues with the agent.

A preferred dosage range for administration to a mammal (e.g., human) of an agonist of the high affinity receptor is the smallest dose that results in a reduction in skeletal muscle or liver lipid of at least about 15%, preferable at least about 25%, more preferably at least about 30%, even more preferably at least about 50%, or more, such as at about 75% (e.g. as measured by a change in fine fatty acid or triglyceride levels).

Another preferred dosage range for administration to a mammal (e.g., human) of an agonist of the high affinity receptor is an amount that results in stimulation of lipolysis in skeletal muscle of a mammal without stimulating, or minimally stimulating, vasodilatation. This amount can be determined any number of ways, such as by (i) conducting dose-response assays by administering a plurality of different doses of a CGRP receptor agonist formulation to test mammals (e.g., by I.V., I.P., oral, or other routes); (ii) measuring the effect of each dose on lipolysis in muscle of the test mammal and measuring the effect of each dose on vasodilatation in the test mammal, thereby creating dose-response data for lipolysis and vasodilatation; and, (iii) determining from the dose-response data a dose of the CGRP receptor agonist formulation that increases lipolysis but does not significantly increase vasodilatation, or only minimally increases vasodilatation, in the mammal. Vasodilatation can measured using any of a variety of assays (see, e.g., Nuki et al., 1993, Biochem Biophys Res Commun 196:245-51). In humans, vasodilatation can be monitored by measurement of systolic blood pressure and mean arterial blood pressure.

Another preferred dosage range for administration to a mammal (e.g., human) of an agonist of the high affinity receptor is an amount that results in stimulation of lipolysis in skeletal muscle of a mammal without stimulating, or only minimally stimulating, an increase in levels of glucose and/or lactate in blood (i.e., a statistically significant increase over baseline levels). This amount can be determined any number of ways, such as by (i) conducting dose-response assays by administering a plurality of different doses of a CGRP receptor agonist formulation to test mammals (e.g., by I.V., I.P., oral, or other routes); (ii) measuring the effect of each dose on lipolysis in muscle of the test mammal and measuring the effect of each dose on glucose and/or lactate levels in the test mammal, thereby creating dose-response data for lipolysis and glucose/lactate levels; and, (iii) determining from the dose-response data a dose of the CGRP receptor agonist formulation that increases lipolysis but does not increase, or only minimally increases, glucose and/or lactate levels in the mammal. Blood glucose and lactate can be measured using routine methods, such as through immobilized enzyme chemistries such as is found in a standard glucose analyzer (glucose oxidase, L-lactate oxidase, Analyzer model 2300-STAT, Yellow Springs Instruments, Yellow Springs, Ohio). Briefly, the substrate is oxidized as it enters the enzyme layer, producing hydrogen peroxide, which passes through cellulose acetate to a platinum electrode where the hydrogen peroxide is oxidized. The resulting current is proportional to the concentration of the substrate. Also see, for example, Ye et al., 2001, Diabetes, 50: 411-417; Ellis et al., 2000, Am. J. Phys. 279: E554-E560.

In the case of naturally occurring CGRP-1 polypeptide (e.g., human CGRP-1) a pharmaceutical formulation that results in a plasma or serum concentration of CGRP-1 in the EC₅₀ range for the high affinity CGRP receptor (e.g., range 10⁻¹¹ M to 10⁻¹³ M) but below the EC₅₀ for the metabolic amylin receptor (e.g., range 10⁻⁹ M to 10⁻⁸ M) is particularly useful. For CGRP-1 and biologically functional variants, an exemplary dose results in a plasma or serum level of between about 10⁻¹⁵ M and about 10⁻¹⁰ M, between about 10⁻¹⁵ M and about 10⁻¹¹ M, or between about 10⁻¹⁵ M and about 10⁻¹² M. Useful plasma or serum level are usually <10⁻¹⁰ M. It will be appreciated these ranges are exemplary and not for limitation. Plasma and serum levels of compounds (including non peptide compounds) can be measured by routine means, such as ELISA, RIA, spectroscopy, enzymatic assays, or other methods).

It will be appreciated that, guided by the teachings herein a variety of methods can be used to identify compounds, compositions, doses, and formulations that preferentially agonize or stimulate the high affinity CGRP receptor compared to the metabolic amylin receptor. It will also be apparent that these methods can be used for screening for agents useful for treatment of insulin resistance and other conditions.

Competitive binding-assays using the recombinant receptor can provide information on the intrinsic binding-affinities (e.g. Kₗ values) of a compound with the metabolic amylin receptor, and demonstrate whether a compound of interest acts through a receptor-mediated mechanism. For example, if compound of interest can block second messenger production by amylin or CGRP at the metabolic amylin receptor, then this is an indication that the compound is an antagonist. If the compound itself can evoke a second messenger response in the absence of amylin or CGRP, then this is an indication that the compound in question is an agonist. Information on relative efficacies of compounds, e.g., as measured by EC₅₀ parameters derived from second messenger (e.g. cAMP) responses at compound concentrations sufficient to saturate the receptor. Suitable concentrations can be determined on the basis of intrinsic binding affinities for the compound.

Exemplary Agonists

A variety of compounds may agonize the high affinity CGRP receptor, including polypeptides and peptidomimetics, and non-polypeptide compounds such as small organic molecules (e.g., molecular weight <1000).

In one embodiment, the high affinity CGRP receptor agonist is a polypeptide. In an embodiment, the polypeptide has a sequence identical to, or with significant sequence similarity to a naturally occurring CGRP-1 polypeptide (e.g., a human CGRP-1 polypeptide or a homolog from rat, pig, cow, rabbit, chicken, salmon, or other species; see Cooper et al., 1994, Endocr Rev. 15:163-201). In this context, a specified polypeptide has significant sequence similarity to a naturally occurring CGRP-1 polypeptide when the specified polypeptide comprises a sequence of amino acids that, when aligned for optimal match, corresponds to at least about 75%, sometimes at least about 80%, and sometimes at least about 90% of the residues of the naturally occurring CGRP-1 polypeptide (e.g., human). In one embodiment, the sequence of the receptor agonist differs from CGRP-1 (e.g., human CGRP-1) only by conservative substitutions.

In a related embodiment, the high affinity CGRP receptor agonist is a biologically functional variant of a CGRP-1 polypeptide, such as a polypeptide or peptide analog (such as a peptidomimetic). Polypeptides that are biologically functional variants of CGRP-1 (including polypeptides with substantial sequence identity and conservatively substituted variants) are characterized by the following properties: (i) they stimulate lipolysis in skeletal muscle, (ii) they have an EC₅₀ for effecting lipolysis stimulation via the metabolic amylin receptor significantly greater than the EC₅₀ for effecting lipolysis stimulation via the high affinity CGRP receptor, i.e., they can preferentially stimulate the high affinity CGRP receptor), and usually they (iii) comprise a sequence of amino acids that, when aligned for optimal match, correspond along the length of the variant to a naturally occurring CGRP-1 sequence, e.g., human CGRP-1 sequence, at least about 60%, sometimes at least about 75%, sometimes at least about 80%, and sometimes at least about 90% of the residues (i.e., substantially similar).

Guided by the present disclosure, variants can be designed and tested for agonist activity. Conventional methods for mutagenesis (e.g., site-directed mutagenesis, alanine scanning and analysis using methods described herein or known in the art, can be used to identify biologically functional variants of CGRP-1. In one aspect, the invention provides a method of making a non-naturally occurring agonist of the high affinity CGRP receptor useful in preparation of a therapeutic agent by (i) obtaining a sequence of a naturally occurring CGRP polypeptide (e.g., human CGRP-1); (ii) modifying at least one amino acid residue by substitution or deletion to create a CGRP variant; (iii) testing the ability of the variant to stimulate one or more activities mediated by the high affinity CGRP receptor (e.g., lipolysis-stimulating activity in skeletal muscle); and (iv) identifying a variant that can preferentially agonize the high affinity CGRP receptor compared to the metabolic amylin receptor as a non-naturally occurring agonist of the high affinity CGRP receptor useful in preparation of a therapeutic composition.

In designing or screening for agonists of the high affinity CGRP receptor, it will also be appreciated by those of skill guided by this disclosure that at least part of the amino-terminal ring structure of naturally occurring CGRP appears to be required for activity (i.e., truncated CGRP peptides lacking the ring structures are antagonists of certain CGRP activities) including high affinity receptor agonist activity. The ²Cys-⁷Cys disulphide bridge (which form a 6 amino acid-containing “ring” structure) and the C-terminal amide are reported to be required for full biologic activity (Cooper et al., 1988, Proc. Natl. Acad. Sci. USA, 85:7763-66). Thus, in an embodiment, biologically functional variants of CGRP-1 that act as agonists of the high affinity CGRP-1 receptor include such an amino acid ring structure, or an equivalent (e.g., a nonpeptide structural homolog), and/or are amidated. In one embodiment, the agonist is a polypeptide having the formula of a reported consensus sequence for the CGRP family: Xxx Cys Xxx Thr Ala Thr Cys Val Thr His Arg Leu Ala Xxx Xxx Leu Xxx Arg Ser Gly Gly Xxx Xxx Xxx Xxx Asn Phe Val Pro Thr Xxx Val Gly Xxx Xxx Ala Phe, where Xxx is any amino acid. See, Cooper et al., Endocr Rev. 1994 15:163-201.

In some embodiments, the agonist is, or corresponds to, a fragment of a CGRP polypeptide (e.g., at least 6 residues, more often at least about 20, 25, 30 or 35 residues). In one embodiment of the invention, the agent has a sequence of a chimeric CGRP polypeptide in which one or more amino acids in a naturally occurring sequence from one species (e.g., human) is replaced with a different amino acid found the corresponding sequence of a CGRP-1 from one or more different species (e.g., rat).

CGRP peptides (e.g., including biological functional variants) can be prepared by routine synthetic or recombinant methods, using known sequences for these proteins. Recombinant techniques and other methods useful in the practice of the present invention are known in the art and are described in, for example, Sambrook and Russel (2001) MOLECULAR CLONING: A LABORATORY MANUAL (3rd Edition) Cold Spring Harbor Laboratory Press; Ausubel et al. (1987) CURRENT PROTOCOLS IN MOLECULAR BIOLOGY (as supplemented through 2001), John Wiley & Sons, New York. De novo chemical synthesis of polypeptides is well known and can be used to prepare CGRP polypeptides (see e.g., Caruthers et al., 1980, Nucleic Acids Res. Symp: Ser., 215-223; and Horn et al., 1980, Nucleic Acids Res. Symp. Ser., 225-232). For example, peptide synthesis can be performed using various solid-phase techniques (Roberge, et al., 1995, Science 269:202), including automated synthesis (e.g., using the Perkin Elmer ABI 431 A Peptide Synthesizer in accordance with the instructions provided by the manufacturer). The newly synthesized peptide can be substantially purified, for example, by preparative high performance liquid chromatography (e.g., Creighton, PROTEINS, STRUCTURES AND MOLECULAR PRINCIPLES, WH Freeman and Co, New York N.Y. [1983]). The amino acid sequence of the CGRP polypeptide, or any part thereof, may be altered during direct synthesis and/or combined using chemical methods with sequences from other proteins (particularly sequences from homologous CGRP polypeptides from the same or other animal species), to produce a variant polypeptide of the invention. Alternatively, CGRP polypeptides can be isolated from natural sources.

Nucleotide and amino acid sequences for CGRP polynucleotides (genomic and cDNA) and polypeptides are known, and can be found in the scientific literature as well as in GenBank (e.g., http://www.ncbi.nlm. nih.gov/Database/index.html) using the search terms “CGRP” and “calcitonin gene-related peptide.” GenBank accession numbers for CGRP polypeptides are provided in Table 2.

TABLE 2
GENBANK ACCESSION NUMBERS FOR CGRP PROTEINS
AND GENES

| Species | GI accession |
|---|---|
| Rattus rattus | 115485, 281020; 203227 |
| Mus musculus | 12744898, 13241720 |
| Gallus gallus | 115539; 2134304; 222802; 1334708; |
| Sheep | 399233; 109012; 256539 |
| Phyllomedusa bicolor | 7387567; |
| Homo sapiens | 115482; 2144644; 179828; 296638; 9945304; |
| | 14762355; 115487; 86999; 9929240; 14250056; |
| | 180466; 14768186; 2134847; 1340176; 179799; |
| | 457134; 223948 |
| Bos taurus | 227702 |
| Oryctolagus cuniculus | 10281574; 227771; 10281572; |
| Rana ridibunda | 399232; 388464 |
| Canis familiaris | 9367718 |
| Equus caballus | 7804969; 7804971 |
| Pig | 399231 |
| Oncorhynchus species | 251851; 1730319 |

It is contemplated that in an embodiment of the methods and various compositions of the invention, the agonist(s), or test compound(s) or agent(s) (e.g., used in screening) is a compound other than CGRP. For example, the invention provides a method for determining whether an agent useful for stimulating lipolysis in a mammal by determining whether the agent is an agonist of the high affinity CGRP receptor. In one contemplated embodiment of this method, the agent is not CGRP.

In one embodiment, the agonist is other than a CGRP-related polypeptide. For example, the agonist can be a polypeptide of unrelated sequence or, alternatively, a non-polypeptide molecule. Exemplary non-polypeptide agonists may be compounds such as carbohydrates such as oligosaccharides and polysaccharides; polynucleotides; lipids or phospholipids; fatty acids; steroids; dipeptides, amino acid analogs, and organic molecules, e.g., small molecules. In certain embodiments, the agonist is other than CGRP-1 of a human or other than a CGRP-1 polypeptide of a mammal.

In one embodiment, a polypeptide or non-polypeptide agonist is prepared using rational drug design methods (e.g., using an integrated set of methodologies that include structural analysis of target molecules, synthetic chemistries, and advanced computational tools). See, e.g., Kim et al., 2000, Comb Chem High Throughput Screen. 3:167-83 and Coldren, 1997, Proc. Natl. Acad. Sci. USA 94:6635-40.

In one embodiment, the agonist is a compound identified according to a screening method described hereinbelow.

In one aspect, the invention provides a method of preparing a pharmaceutical composition by preparing the agonist as above, and combining the agonist with a pharmaceutically acceptable excipient. In certain embodiments, the pharmaceutical composition is formulated as sterile, substantially isotonic and in full compliance with all Good Manufacturing Practice (GMP) regulations of the U.S. Food and Drug Administration.

In a different aspect, the invention provides methods for stimulation of lipolysis by stimulation of either the high affinity CGRP receptor, the metabolic amylin receptor, or both, using CGRP-1 or polypeptides that are metabolic receptor stimulating variants of CGRP-1, based on the discovery that CGRP-1 binding to the metabolic amylin receptor stimulates lipolysis. Often there is a further step of monitoring a change in the level of lipolysis in a tissue of the mammal (e.g., by measuring the amount of free fatty acids in muscle, liver, blood or other tissue of the mammal) or isolated tissue. Metabolic receptor stimulating variants of CGRP-1 are characterized by the following properties: (i) they stimulate lipolysis in skeletal muscle, and (ii) they comprise a sequence of amino acids that, when aligned for optimal match, correspond along the length of the variant to a naturally occurring CGRP-1 sequence, e.g., human CGRP-1 sequence, at least about 60%, sometimes at least about 75%, sometimes at least about 80%, and sometimes at least about 90% of the residues. In an aspect the invention provides a method of stimulating lipolysis in skeletal muscle or liver of a mammal by contacting the skeletal muscle or liver with CGRP-1 or a metabolic receptor stimulating variant thereof.

In an aspect, the invention provides a therapeutic regimen including (i) administering a CGRP-1 polypeptide, a biologically function variant thereof, or a metabolic receptor stimulating variant thereof to a mammal suffering from or susceptible to a condition characterized by accumulation of lipid in skeletal muscle, and (ii) monitoring lipolysis in the mammal.

### D. Administration and Dosage Forms Agonists

In one aspect, according to the invention, a therapeutically effective amount of an agonist of the high affinity CGRP receptor (e.g., a CGRP-1 polypeptide) is administered to a subject (e.g., patient or animal) who would benefit from a reduction in tissue lipid content (e.g., skeletal muscle lipid content). As discussed supra, such reduction in lipid content is beneficial for subjects with a variety of conditions, e.g., insulin resistance, type-2 diabetes mellitus, hypertension, obesity, dyslipidaemia, atherosclerosis, and thrombosisais. The dosage ranges for the administration of the agents of the invention are those large enough to produce the desired effect (e.g., reduction in lipid content or amelioration of symptoms or progression of the condition).

In a related aspect, the invention provides an agonist of the high affinity CGRP receptor in a unit dosage form for administration to patients. As used herein, “unit dosage form” refers to a composition intended for a single administration to treat a subject suffering from a disease or medical condition. Each unit dosage form typically comprises each of the active ingredients of this invention plus pharmaceutically acceptable excipients. Examples of unit dosage forms are individual tablets, individual capsules, bulk powders, liquid solutions, suppositories, emulsions or suspensions. Treatment of the disease or condition may require periodic administration of unit dosage forms, for example: one unit dosage form two or more times a day, one with each meal, one every four hours or other interval, or only one per day. The expression “oral unit dosage form” indicates a unit dosage form designed to be taken orally. Formulations for injection may be presented in unit dosage form, e.g., in ampules or in multidose containers.

The unit dosage form of the invention contains a therapeutically effective dose of the receptor agonist. In an embodiment, administration of the unit dosage form results in a level of agonist in the mammal to preferentially stimulates the high affinity receptor (compared to the metabolic amylin receptor). Thus, in one embodiment of the invention, the agonist of the high affinity CGRP receptor (e.g., CGRP-1 protein or biologically functional variant thereof) is administered at a concentration that does not saturate binding to the metabolic amylin receptor (e.g., as monitored through effects known to occur through the metabolic amylin receptor, such as increases in blood or plasma lactate and glucose levels). In an embodiment, the dose results in only minimal increase (if any) in blood or plasma lactate and glucose levels or vasodilatation.

Although the particular dose will depend on the molecular structure and chemical properties of the particular agonist, those of skill in the pharmacology art will understand from the disclosure herein that appropriate doses can be determined using routine techniques. For example, a dose or formulation of an agonist of the high affinity CGRP receptor that stimulates lipolysis in skeletal muscle of a mammal without or only minimally eliciting an undesired side-effect in the mammal (e.g., an increased level of in blood glucose, blood lactate, or vasodilatation in the mammal) can be determined in a variety of ways. As used in this context, “an increased level” can refer to an increase to a predetermined level (e.g., a designated threshold level of the sideeffect). One method for making such determination involves) conducting dose-response assays by (a) administering a plurality of different doses (or formulations) of the CGRP receptor agonist to test mammals; and (b) measuring the effect of each dose or formulation on lipolysis in a tissue of the test mammal and measuring the effect of each dose on the side-effect, thereby creating dose-response data for lipolysis and the side-effect; and, (ii) determining from the dose-response data a dose of the CGRP receptor agonist formulation that increases lipolysis but does not elicit the side-effect. In an embodiment, the effect of each dose or formulation on lipolysis is determined by measuring free fatty acid levels in a tissue of the animal. Analogous methods can be carried out using isolated tissues in place of whole animals. Further, based on the guidance herein, it will be within the ability of one of ordinary skill to determining the desired dose using any of a variety of methods.

Usually the agonist doses for non-polypeptide compounds would fall in a concentration of from 10⁻⁶ M to 10⁻⁵ M (e.g., as measured in one or more of muscle, blood, serum or plasma). As noted above, in the case of CGRP-1 polypeptide (e.g., human CGRP-1) a dose that results in a plasma or serum concentration of CGRP-1 in the EC₅₀ range for the high affinity CGRP receptor (10⁻¹¹ M to 10⁻¹³ M) but below the EC₅₀ for the metabolic amylin receptor (10⁻⁹ M to 10⁻⁸ M) is particularly useful. For CGRP-1 and biologically functional variants, an exemplary dose results in a serum or plasma level of between about 10⁻¹⁵ M and about 10⁻¹⁰ M. In one embodiment, the serum level is less than 300 pM.

The amount of an agent administered to an animal to achieve a desired level or concentration of agent will depend on a number of factors well know to practicioners, such as compound half-life (e.g., serum half-life), and the frequency and mode of administration. For illustration and not limitation, the the dose of a CGRP-1 polypeptide is administered in the range from 20 picograms to 1 gram, more often between 3 nanograms and 50 micrograms daily. In various embodiments, the unit dosage (in some cases daily dosage) is less than about 10 micrograms, less than about 1 microgram, less than about 100 nanograms, less than about 10 nanograms, less than about 1 nanogram, less than about 100 picograms, or less than about 10 picograms.

Other ranges of both polypeptide and non-polypeptide compounds will be apparent to the skilled practitioner based on data from initial dose-response curves, as described above, and other data that can be obtained by routine methods.

The invention also provides a composition containing an agonist of the high affinity CGRP receptor combined with a pharmaceutically acceptable excipient. In one embodiment, the agent and excipient are formulated to selectively activate a high affinity CGRP receptor without activating an amylin receptor. In another embodiment, the agent and excipient are formulated to selectively stimulate lipolysis without, or only minimally, stimulating adverse side effects such as vasodilatation, or elevation of blood glucose or lactate levels.

The CGRP polypeptide or other agonist can be formulated or coadministered with other active agents (e.g., agents that alone or in combination CGRP, reduce lipid, free fatty acid, and/or long chain CoA levels). It is contemplated that, in an embodiment, the agonist of the present invention is not coadministered with insulin. In one aspect, the agonist of the high affinity receptor is admininstered with an agent that inhibits activation of the metabolic amylin receptor but not the high affinity receptor (e.g., an anti-receptor antibody).

Agonists (or antagonists) used in the practice of the invention can be directly administered to the host to be treated. Administration is optionally under sterile conditions. However, while it is possible for the active ingredient to be administered alone, it is often preferable to present it as a pharmaceutical formulation. Formulations typically comprise at least one active ingredient together with one or more acceptable carriers thereof. Each carrier should be both phannaceutically and physiologically acceptable in the sense of being compatible with the other ingredients and not inurious to the subject. Therapeutic formulations can be prepared by any methods well known in the art of pharmacy. See, e.g., Gilman et al (eds.) (1990) GOODMAN AND GILMAN'S: THE PHARMACOLOGICAL BASES OF THERAPEUTICS (8TH ED.) Pergamon Press; and (1990) REMINGTON, THE SCIENCE OF PRACTICE AND PHARMACY, 20TH EDITION, Mack Publishing Co., Easton, Pa.; Avis et al (eds.) (1993) PHARMACEUTICAL DOSAGE FORMS: PARENTERAL MEDICATIONS Dekker, N.Y.; Lieberman et al. (eds.) (1990) PHARMACEUTICAL DOSAGE FORMS: TABLETS Dekker, N.Y.; and Lieberman et al (eds.) (1990) PHARMACEUTICAL DOSAGE FORMS: DISPERSE SYSTEMS Dekker, N.Y.

The compounds of the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. When the agonist is a polypeptide, administration will often be parenteral (e.g., intravenous).

If desired (e.g., to maintain a particular plasma concentration) agonist can be administered to patients in the form of controlled delivery formulations. A variety of suitable controlled delivery systems are known, including forms suitable for oral, parenteral, and other routes of administration. See, e,g, Bogner et al., 1997 U.S. Pharmacist 1997;22(Suppl.):3-12; and GUIDANCE FOR INDUSTRY. EXTENDED RELEASE ORAL DOSAGE FORMS: DEVELOPMENT, EVALUATION, AND APPLICATION OF THE IN VITRO/IN VIVO CORRELATIONS. Rockville, Md.: Center for Drug Evaluation and Research, Food and Drug Administration, 1997. Examples of controlled drug formulations can be found in standard references (for example, see: Sweetman, S. C. (Ed.). Martindale. THE COMPLETE DRUG REFERENCE, 33rd Edition, Pharmaceutical Press, Chicago, 2002, 2483 pp.; see also: Aulton, M. E. (Ed.) PHARMACEUTICS. THE SCIENCE OF DOSAGE FORM DESIGN. Churchill Livingstone, Edinburgh, 2000, 734 pp.; see also: Ansel, H. C., Allen, L. V. and Popovich, N. G. PHARMACEUTICAL DOSAGE FORMS AND DRUG DELIVERY SYSTEMS, 7th Ed., Lippincott 1999, 676 pp.). Excipients employed in the manufacture of drug delivery systems are described in various publications known to those skilled in the art (for example, see: Kibbe, E. H. HANDBOOK OF PHARMACEUTICAL EXCIPIENTS, 3rd Ed., American Pharmaceutical Association, Washington, 2000, 665 pp.). The USP provides many examples of modified-release oral dosage forms (for example, see: The United States Pharmacopeia 23/National Formulary 18, The United States Pharmacopeial Convention, Inc., Rockville Md., 1995). This publication also presents general chapters and specific tests to determine the drug release capabilities of extended-release and delayed-release tablets and capsules. In one aspect of the invention, the agonist is administered in conjunction with a program of exercise, to enhance exercise-mediated breakdown of triglycerides in a subject.

The pharmaceutical compositions may be in the form of a sterile injectable aqueous or oleagenous suspension. This suspension may be formulated according to the known art using those suitable dispersing or wetting agents and suspending agents. The sterile injectable preparation may also be a sterile injectable solution or suspension in a non-toxic parenterally-acceptable diluent or solvent. Among the acceptable vehicles and solvents that may be employed are water, Ringer's solution and isotonic sodium chloride solution. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, and the severity of the particular condition. In some embodiments, daily or weekly administration of the agonist is contemplated.

### E. Monitoring of Lipid Conent, Blood Glucose Levels, Blood Lactate Levels, Vasodilatation, Insulin Resistance and Other Conditions

In some embodiments of the invention, a CGRP receptor agonist is administered to a mammal and the mammal is monitored for stimulation of lipolysis or reduction in lipid content in the mammal or in a tissue of the mammal (e.g., in skeletal muscle of the mammal). Lipolysis is usually monitored by detecting a change in amount or composition of free fatty acids in a target tissue (e.g., skeletal muscle, blood, blood plasma). Methods for detecting a change in free fatty acid levels are well known. For example, as described in Example 1, infra, gas chromatographic analysis of muscle tissue (obtained from biopsy or tissue culture) can be conducted. Other methods include determination of plasma non-esterified fatty acids (NEFA) by an acyl CoA oxidase-based calorimetric assay (Wako Pure Chemical Industries, Osaka, Japan). Plasma triglycerides can be measured using a colorimetric assay (Triglyceride Procedure 336, Sigma Diagnostics). Recent evidence also suggests that long-chain acyl CoA esters may serve as markers of lipid metabolism and insulin sensitivity in rat and human muscle (see Bronwyn et al., 2000, Am. J. Phys. 279: E554-E560). Tissue long chain CoA (e.g. derived from muscle biopsies) can be measured by measured by solvent extraction of long chain CoA from tissues, phase separation, and purification by reverse phase high performance liquid chromatography (see for example, Bronwyn et al., 2000, Am. J. Phys. 279:E554-E560). In addition, new methodologies for the measurement of intramuscular triglyceride content may be used. These include non-invasive methods such as computed tomography and nuclear resonance spectroscopy (see for example, Kelley et al, 1991, J. Clin. Nutr. 54: 509-515; Krssak et al, 1999, Diabetologia 42: 113-116; Jacob et al., 1999, Diabetes, 48: 1113-1119).

In some embodiments of the invention, a CGRP receptor agonist is administered to a mammal and vasodilatation in the mammal is measured. CGRP-1 and CGRP-2 are potent vasodilators (see, e.g., Muff et al., 1995, Eur. J. Endocrinol. 133:17-20; Nuki et al., supra). However, it is postulated the skeletal muscle lipolysis-stimulating effects of CGRP-1 (and other agonists of the high affinity CGRP receptor) can be at least partially uncoupled from the vasodilatation effects, e.g., by administration of an agent of a type, formulation, or dose that agonizes the high affinity receptor but does not agonize (or minimizes activation of) the metabolic amylin receptor. In an embodiment of the invention, the agent is administered to a mammal and vasodilatation in the mammal is measured. Any suitable assay for vasodilatation can be used (see, e.g., Nuki et al., 1993, Biochem Biophys Res Commun 196:245-51). In humans, vasodilatation can be monitored by measurement of systolic blood pressure and mean arterial blood pressure.

In some embodiments of the invention, a CGRP receptor agonist is administered to a mammal and blood glucose production and/or lactate levels are measured. As noted above, lipolysis mediated by the high affinity receptor can be achieved without the undesirable effects mediated by the metabolic amylin receptor (e.g., increased blood glucose levels)

In some embodiments of the invention, a CGRP receptor agonist is administered to a mammal and the mammal is monitored for alleviation of symptoms of a disorder associated with muscle fat accumulation. In some aspects of the invention, the course of a disease is monitored at least long enough to determine whether there is a reduction in severity, symptoms, or progression of the condition. In some embodiments of the invention, a CGRP receptor agonist is administered to a mammal (e.g., a mammal in need of lipolysis stimulation) and one or more disease or physiological conditions in the mammal are monitored. For example, in some embodiments of the invention, a CGRP receptor agonist is administered to a mammal, and the mammal is monitored for the effect of administration of the agonist on insulin resistance. Insulin resistance in an animal can be assessed by any of a variety of methods known in the art. For example, in humans, insulin resistance can be monitored using an oral glucose tolerance test or OGTT (see, e.g., Bergman et al., 1985, Endocrinology Review 6:45-86). In general, individuals with 75 gram, 2 hour OGTT level greater than 140 mg/dl are considered insulin resistant, and individuals a level less than 120 mg/dl are considered normal. Insulin resistance is also be monitored using a steady state blood glucose test (see, e.g., Reaven et al., 1979, Diabetologia 16:17-24). Individuals with SSPG mean greater than 180 mg/dl are usually considered a insulin resistant; individuals with SSPG mean less than 150 mg/dl are considered normal. Other methods include measurement of HbAlc and C-peptide as markers for insulin resistance (del Prato, 1999, Drugs 58:Supp 1:3-6; Ferranini et al., 1998, Hypertension 16:895-906).

Any of the above-mentioned monitoring activities can be carried out in combinations; thus, for example, in one embodiment, both vasodilatation and insulin resistance are monitored in the mammal to which the agonist is administered. In embodiments in which a condition (e.g., insulin sensitivity/resistance) or effect of agonist administration (e.g., lipolysis) is monitored, measurements can be made prior to administration of the agent (e.g., to generate a baseline) and/or at one or more time points after administration (or, in the case of multiple administration, after one or more administrations).

In embodiments of this invention the agonist is administered to an individual diagnosed with insulin resistance. In one embodiment, the individual is diagnosed as suffering from insulin resistance, but not diagnosed as diabetic (e.g., suffering from type II diabetes).

Provided with the guidance herein, e.g., disclosing that the lipolysis and vasodilatation activities of CGRP (or other CGRP receptor agonists) can be at least partially uncoupled, it will be apparent to the skilled practitioner that by monitoring vasodilatation and a desired outcome (increased lipolysis, increased insulin sensitivity, and the like) a mammal can be administered an amount of CGRP or other agonist sufficient to stimulate lipolysis (and thereby reduce muscle fat accumulation) without eliciting, or only minimally eliciting, increased vasodilatation.

### F. Screening Methods

In an aspect, the invention provides a method for determining whether an agent is or is not useful for stimulating lipolysis in a mammal or mammalian cell, e.g., in skeletal muscle in a mammal, by determining whether the agent is an agonist of a CGRP receptor. In one embodiment, agents that preferentially activate the high affinity CGRP receptor (compared with the metabolic amylin receptor) are identified. As noted above, such agents are useful in making therapeutic compositions and in treatment of diseases and conditions. As described hereinbelow, preferential stimulation of the high affinity receptor can be determined in a variety of ways, including by comparing the EC₅₀ of the agent for effecting a response mediated by the metabolic amylin receptor (e.g., an effect on carbohydrate metabolism) and the EC₅₀ of the agent for effecting a response mediated by the high affinity CGRP receptor (an increase in free fatty acids in skeletal muscle tissue or a skeletal muscle cell).

It will be understood that the identification and screening methods described herein can be used to screen a single compound or a plurality of compounds for therapeutically useful activity. The plurality may be at least 3 agents, more often at least 25, even more often at least 50, usually at least 100, and may involve screening a very large number (>1000) of agents. Screens may be high-throughput and/or may involve assays carried out sequentially or simultaneously (including beginning the assay for one agent after starting but before completing the assay for another agent).

Any suitable assay can be used for identifying preferential activation including (but not limited to) identification on the basis of the EC₅₀ of the agent for stimulating lipolysis via the high affinity receptor (measured, for example, by a reduction in tissue triacylglceride content or increase in free fatty acid) compared to the EC₅₀ (if any) of the agent for stimulating lipolysis via the metabolic amylin receptor and/or identification using other assays described herein (see, e.g., § II(B) and examples). It will be recognized that these assays can be used to screen multiple agents, for example, a library of compounds, e.g., in a high-throughput format. It will be appreciated that, guided by the teaching herein of the existence of, and differential effects of, the high affinity CGRP receptor, a variety of methods can be used to identify compounds, compositions, doses, and formulations that preferentially agonize or stimulate the high affinity CGRP receptor compared to the metabolic amylin receptor. It will also be apparent that these methods can be used for screening for agents useful for treatment of insulin resistance and other conditions.

Screening assays of the invention can be conducted, without limitation, using animals, in vitro tissue or organ culture (e.g., isolated skeletal muscle), cells or cell lines, for example, to determine whether the compound is useful for reduction in lipid content in an animal, cell or tissue. Alternatively, the agonist is administered to an isolated cell line. See, e.g., Examples, infra.

In one embodiment, the invention provides a method of determining whether an agent is useful for stimulating lipolysis in a mammal by (i) identifying at least one agent, and usually a plurality of agents, that bind the high affinity CGRP receptor (e.g., using CGRP-1 displacement assays); and (ii) selecting an agent that preferentially stimulates the high affinity CGRP receptor compared to the metabolic amylin receptor. In a related embodiment, the invention provides a method of determining whether an agent is useful for stimulating lipolysis in a mammal by (i) identifying at least one agent, and usually a plurality of agents, that stimulate lipolysis in a tissue expressing the high affinity CGRP receptor; and, (ii) selecting an agent from (i) that preferentially stimulates the high affinity CGRP receptor compared to the metabolic amylin receptor. In one embodiment, the method involves (i) at least one agent, and usually a plurality of agents, that bind the high affinity CGRP receptor; and, (ii) selecting an agent from (i) that preferentially stimulates the high affinity receptor compared to the metabolic amylin receptor. In an embodiment, the tissue is skeletal muscle (e.g., from rat) In one embodiment, the method involves (i) at least one agent, and usually a plurality of agents, that bind the high affinity CGRP receptor; and, (ii) selecting an agent from (i) that has an EC₅₀ for effecting a response mediated by the metabolic amylin receptor which is higher than the EC₅₀ of the agent for effecting a response mediated by the high affinity CGRP receptor. In an embodiment, the tissue is skeletal muscle (e.g., from rat).

In another embodiment, the invention provides a method of determining whether an agent is useful for stimulating lipolysis in a mammal on the basis of the EC₅₀ of the agent for effecting a response mediated by the metabolic amylin receptor and the high affinity CGRP receptor, usually lipolysis, as described hereinabove. In an embodiment, the EC₅₀ of the agent for effecting a response mediated by the metabolic receptor is at least about 10-fold higher, at least about 100-fold, at least about 1000-fold and preferably at least about 10,000-fold higher, at least about 100,000-fold higher, or at least about 1,000,000-fold higher than the EC₅₀ for effecting the response via the high affinity receptor.

In one embodiment, soleus muscle (e.g., isolated soleus muscle) is used for determining the lipolytic activity of a compound. (Lipolytic activity, as used in this context, refers to the ability of an agent to stimulate lipolysis in a cell or tissue.) Soleus muscles can be derived from animals that have been fed a high fat or normal diet and dose-response curves for a compound prepared in which (i) assays reflective of processes that can occur through both the high affinity CGRP receptor and the metabolic amylin receptor (e.g., measurement of muscle free fatty acid and triglyceride content), and (ii) reflective of processes occurring through the metabolic amylin receptor (e.g., measurement of muscle glycogen content and [¹⁴C]-glucose incorporation into glycogen in the presence of maximally-stimulating insulin) are made. The EC₅₀ values (e.g., relative EC₅₀ values) can be determined from the dose-dependent curves. The dose of the compound will be chosen from results of (i) and (ii). Specifically of interest will be the concentration of compound that elicits metabolic effects in (i) but not in (ii). In the comparison of different compounds, this concentration will be a measure of relative compound efficacy. For example, a compound will be defined as having a high relative efficacy over another compound if, on the basis of its EC₅₀ values, it evokes effects in (i) but not in (ii) at a lower relative concentration.

In a related aspect, identification of agents that preferentially activate the high affinity CGRP receptor (compared with the metabolic amylin receptor) includes the step of detecting differential binding to the high affinity CGRP receptor (e.g., the skeletal muscle CGRP receptor) and the metabolic amylin receptor (e.g., the skeletal muscle amylin receptor). Binding studies can be performed conveniently through radioligand binding experiments using a number of several approaches. For example, the test agent is radiolabeled with a suitable radioprobe (e.g. ¹²⁵I or ³H) and incubated with preparations (e.g. skeletal muscle membranes) containing the high affinity CGRP receptor in the presence and absence of excess and unlabelled ligand. A saturation curve is then constructed and the binding parameter, K_{d} (the concentration of test agent required for half-saturation) derived for specific binding. In another example, specific binding parameters of the test agent with the high affinity CGRP receptor can be determined through competition displacement of radiolabeled CGRP ligand. In this case, radiolabeled CGRP is incubated with incubated with preparations (e.g. skeletal muscle membranes) containing the high affinity CGRP receptor in the presence of increasing amounts of unlabelled test agent. A plot of bound CGRP ligand against the concentration (log) of unlabelled test agent will yield a sigmoidal curve from which a inhibition constant, Ki, can be derived for the test agent. Methods for carrying out binding assays and analyzing responses and data are well known in the art. See, for example, www.graphpad.com/prism/, and THE RBI HANDBOOK OF RECEPTOR CLASSIFICATION AND SIGNAL TRANSDUCTION, K. J. Watling, J. W. Kebebian, J. L. Neumeyer, eds. Research Biochemicals International, Natick, Mass., 1995, and references therein. Methods of analysis can be found in T. Kenakin, PHARMACOLOGIC ANALYSIS OF DRUG-RECEPTOR INTERACTIONS, 2nd Ed. Raven Press, New York, 1993, and references therein.

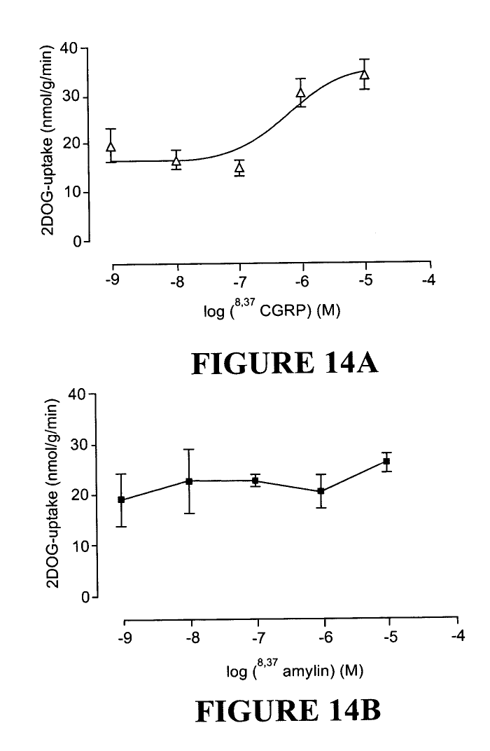

In one aspect of the invention, a high affinity CGRP receptor agonist (or a test compound that may be an agonist) is administered to animals, in a screening assay to determine whether the compound is useful for reduction in lipid content in an animal (e.g., animal tissue). For example, the agonist can be administered to a non-human animal that serves as a model for human diseases or conditions associated with muscle fat accumulation. Examples of such experimental models include animal models for insulin resistance, e.g., the ob/ob (Kreutter, 1991 Diabetes 40[Suppl 1]:159A (Abstract); Bretherton, Endocrinol 129[Suppl]:91A (Abstract)), the db/db (Kreutter, 1991 Diabetes 40 [Suppl 1]:159A (Abstract)), and obese-diabetic viable yellow (Gill, 1991 Life Sci 48:703-710) strains of mice, and the LA/N-cp (Huang, 1992 Hypertension 19[Suppl 1]:101-109) (FIG. 14), the SHRN-cp (Dunning, 1991 Diabetes 40[Suppl 1]), obese Zucker (Kotanyi L 1992 Diabetes 41:685-680), and obese-diabetic Zucker (Geduolin, 1991 Biochem Biophys Res Commun 180:782-789) strains of rat, as well as in animals in which insulin resistance has been induced by administration of glucocorticoids (Jamal H. 1990 Endocrinol 126:425-429), gold thioglucose (Tokuyama, 1991 Endocrinology 128:2739-2744), or lesioning of ventromedial hypothalamus (Tokuyama, 1992 Diabetes Res Clin Pract 15:23-29); Tokuyama, 1991 Endocrinology 128:2739-2744). In addition to lipid content (e.g., appearance of FFA) effects such as blood glucose and lactate levels, and degree of vasodilatation can be assessed to identify compounds that do not cause undesirable effects. It will be appreciated that information from tissue and cell-based assays can be used to determine compound concentrations most useful for conducting ex vivo dose-response studies. Whole animal screening studies can also be used in assessing compounds or doses. Exemplary protocols are analogous to those described for amylin by Ji-Ming et al., 2001, Am. J. Physiol. Endocrinol. Metab. 280:E562-569. It will be understood that, in certain embodiments of the assays described herein, the experimental animal may be sacrificed during or following the experiment.

When the EC₅₀ of an agent (or mixture of test agents, e.g., in some high throughput screening formats) for the high affinity CGRP receptor and/or metabolic amylin receptor is to be determined, a variety of assays can be used. For example, an EC₅₀ for a specified compound, receptor and effect can be determined by assaying the effect of various concentrations of the ligand on binding of CGRP-1 to the high affinity CGRP receptor and generating a dose-response curve of agent concentration versus fatty acid release. In one embodiment, in the case of the high affinity CGRP receptor, various concentrations of the test agent are incubated with isolated soleus muscle strips under conditions that maintain tissue viability. After a defined incubation period, triglyceride and free fatty acids are extracted, quantitated, and plotted against the logarithm of the test agent concentration. A sigmoidal curve is fitted to the data and a EC₅₀ value derived from the concentration of test agent required for 50% maximal stimulation. An exemplary assay and resulting dose-response data are described in the examples, infra. See Example 4. In general, a range of concentrations of CGRP can be added to a sample containing a population of the receptors (e.g., skeletal muscle). By plotting CGRP concentration vs. an indicator of an effect of CGRP binding (e.g., an increase in free fatty acid concentration), the EC₅₀ for CGRP binding can be determined as the CGRP concentration that causes half-maximal stimulation of the effect being determined, e.g., free fatty acid production.

In one embodiment, an agent (or plurality of agents, for example, in pools or in a high throughput screening format as described infra) is (a) assayed to determine the EC₅₀ of the agent for stimulating lipolysis via the high affinity CGRP receptor (e.g., the skeletal muscle CGRP receptor), i.e., “EC_{50-CGRP-R}” and (b) assayed to determine the EC₅₀ for stimulating lipolysis, i.e., “EC_{50-amylm-R}” The two EC₅₀'s are compared and agents with a EC_{50-amylin-R}/EC_{50-CGRP-R} ratio greater than about 10 are considered to have a significantly lower EC₅₀ for the high affinity receptor than the amylin receptor. Preferably the EC_{50-amylin-R}/EC_{50-CGRP-R} ratio is at least about 10, often at least about more than about 10², more than 5×10², more than about 10³, more than 5×10³, more than about 10⁴, more than about 10⁵, more than about 10⁶, more than about 10⁷, or more than about 10⁸.

Screening can be carried out using a variety of types of compounds and compositions. Suitable test agents include naturally occurring and synthetic compounds or compositions. Examples of suitable test agents include polypeptides (including proteins and short peptides, such as the CGRP-related peptides described herein), carbohydrates such as oligosaccharides and polysaccharides; polynucleotides; lipids or phospholipids; fatty acids; steroids; dipeptides, amino acid analogs, organic molecules, e.g., small molecules (e.g., MW less than 1000). The test compounds can be of a variety of chemical types including, but not limited to, heterocyclic compounds, carbocyclic compounds, -lactams, polycarbamates. In one embodiment, the agent is identified by screening libraries of compounds. The creation and screening of large libraries of synthetic molecules can be carried out using well-known techniques in combinatorial chemistry, for example, see van Breemen, 1997, Anal Chem 69:2159-64; Lam, 1997, Anticancer Drug Des 12:145-67; Gold, 1995, J. Biol. Chem. 270:13581-84). In addition, a large number of potentially useful activity-modifying compounds can be screened in extracts from natural products as a source material. Sources of such extracts can be from a large number of species of fungi, actinomyces, algae, insects, protozoa, plants, and bacteria. Those extracts showing activity can then be analyzed to isolate the active molecule. See for example, Turner, 1996, J. Ethnopharmacol 51:3943; Suh, 1995, Anticancer Res. 15:233-39. Many other test agents and libraries are known in the art. In many embodiments, a library of test agents contains at least 100 different test compounds or agents.

In an aspect, the invention provides a method for determining whether an agent is useful for stimulating lipolysis in a mammal by comparing the lipolytic activity of the agent with the lipolytic activity of CGRP. According to this assay, agents that stimulate lipolysis in a cell or tissue (e.g., skeletal muscle or liver) as well or better than CGRP (when compared on an equal molar basis) are considered useful for stimulating lipolysis in a mammal and are candidate compounds for animal or human therapeutics. The step of comparing may be carried out in parallel (done at the same time) assays. Alternatively, the lipolytic activity (e.g., FFA produced per mole agent per unit time under specified conditions) is compared to a standard value previously recorded (e.g., in a computer readable medium or otherwise). The comparison may also be indirect: For example, the lipolytic activity of compound A may be compared to CGRP, and the lipolytic activity of the test agent may be compared with that of compound A (and thus compared indirectly with CGRP). In one aspect the lipolytic activity of CGRP as mediated by the high affinity receptor is used as the basis for comparison. For example, in one embodiment, the comparison is to the lipolytic activity of CGRP at a concentration less than 300 pM. For example, in one embodiment, the comparison is to the lipolytic activity of CGRP at a concentration that does not substantially stimulate lipolysis of the metabolic amylin receptor. In another aspect, the lipolytic activity of CGRP as mediated by both the high affinity receptor and the metabolic receptor, or by the metabolic receptor alone is used as the basis for comparison. In one embodiment, any form of CGRP that stimulated lipolysis in skeletal muscle may be used; in a related embodiment (as will be apparent from the foregoing) the form of CGRP is one that stimulates lipolysis via the high affinity receptor. Exemplary forms of CGRP, without limitation, are rat and human CGRP-1.

### G. Methods of Inhibiting Lipolysis

In another aspect, the invention provides a method of inhibiting lipolysis in skeletal muscle by administering an antagonist of the high affinity and/or metabolic receptor(s). Inhibition of lipolysis in tissues is useful in both experimental and clinical settings. For example, chronic administration of antagonists may induce compensatory metabolic processes in muscle and liver that lead to increased lipid utilisation (e.g. upregulation of β-oxidation) and result in a desirable reduction in muscle lipid. Agents that inhibit lipolysis are also used in drug screening assays (e.g., as positive and negative controls for identification of lipolysis modulators). The antagonist may be any compound that inhibits lipolysis stimulated by an agent such as CGRP or amylin. The antagonist may inhibit binding or compete for binding to the high affinity CGRP receptor or the lower affinity metabolic amylin receptor (see, e.g., Example 4 infra). The antagonist may be a naturally-occurring compound, such as a protein, peptide, polynucleotide, or small effector molecule, or may be synthetically produced. In one embodiment the antagonists is ^{8,37}amylin or ^{8,37}CGRP. ^{8,37}CGRP and ^{8,37}amylin refer to variants of CGRP and amylin, respectively, in which the C-terminal first seven amino acids have been removed. (See, Cooper, 2001, “The endocrine pancreas and regulation of metabolism” in HANDBOOK OF PHYSIOLOGY, II(7): The Endocrine System, Jefferson, L. S. Cherrington, A. D., eds., Oxford University Press). Non-peptide CGRP antagonists may also be used. Examples include BIBN4096BS (see, Wu et al., 2002, Biochem Soc. Trans. 30:468-73), “compound-1” and “compound-2” (see Mallee et al., 2002, J Biol Chem. 277:4294-98).

The antagonist may be administered in a therapeutically effective amount to a mammal in need of lipolysis inhibition. The receptor antagonists can be combined with a pharmaceutically acceptable excipient and administered to an animal. Modes of administration are similar to those described, supra, for administration of a receptor agonist.

### EXAMPLES

### Example 1

### Materials and Methods

This example describes materials and methods used in the experiments described in Examples 2-7.

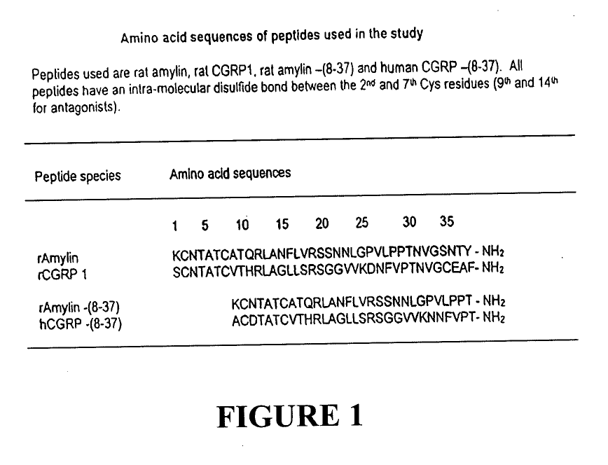

Materials. All chemical were of analytical grade or better, unless otherwise stated. Nembutal was from Virbac Laboratories Ltd. Rat amylin, rat amylin-(8-37), rat CGRP1 and human CGRP1-(8-37) (see FIG. 1) were from Bachem, Switzerland. NaHCO₃, CaCl₂, D-glucose, NaCl, KH₂PO₄, chloroform, methanol, HCl, NH₄OH, ethanol and Triton X-100 were from BDH; MgSO₄.7H₂O from Sigma; and KCl from Riedel-de Haen. Teflon columns (16 ml), teflon frits and Vac Elut apparatus were from Alltech. Aminopropyl bonded silica 40μ (Bond Elut) column packing was from Phenomenex; hexane and diethyl ether were from Labscan. Heptane was from Waters Associates Inc and boron trifluoride-methanol complex (BF3 in methanol) was from Aldrich Chemical Company Inc. Butylated hydroxy toluene and all fatty acid methyl ester standards were purchased from Sigma. GLC column DB 225 (30 m×25 mm I.D.) was obtained from J & W Scientific. Tri-n-octylamine was from Acros Organics; trichloroacetic acid was from Ajax Chemicals Ltd; and glycerol was from Scharlau. Free fatty acids, Half-micro test was from Boehringer Mannheim. Lipase Free Reagent powder for glycerol tests and triglyceride tests were purchased from Pointe Scientific Inc.

Animal models. Normallyfed rats. All protocols were approved by the University of Auckland Animal Ethics Committee. Male Wistar rats, (age=50 d) weighing 200-250 g, were housed in a constant 12 h/12 h-light/dark cycle and received standard rat-chow (Diet 86, NRM, Auckland) and water ad libitum. The standard diet contained 5% fat (w/w), which comprised mainly beef tallow. The triglyceride, total and free fatty acid contents of representative samples of this diet were analyzed, and are shown in Table 3. Rats were anaesthetized by intraperitoneal injection of sodium pentobarbital (45 mg/kg body weight) then sacrificed by cervical dislocation.

TABLE 3
FATTY ACID COMPOSITION OF STANDARD RAT CHOW
Free fatty acid and total lipid samples were analyzed by gas
chromatography, with confirmation of the identity of individual
fatty acids by mass spectroscopy, to determine the free fatty acid
and total fatty acid composition of standard rat chow. Values are
means ± S.E.M. (n = 7 at each point)

| Fatty Acid | | | FFA (μmol/g) | FA (μmol/g) |
|---|---|---|---|---|
| myristic | | C14:0 | 10.52 ± 2.44 | 12.54 ± 3.26 |
| pentadecanoic | | C15:0 | 7.93 ± 1.44 | 8.90 ± 0.324 |
| palmitic | | C16:0 | 508 ± 97.8 | 610 ± 21.8 |
| palmitoleic | (n-9) | C16:1 | 7.10 ± 1.30 | 8.91 ± 0.469 |
| palmitoleic | (n-7) | C16:1 | 33.0 ± 6.14 | 39.1 ± 1.48 |
| margaric | | C17:0 | 9.57 ± 2.12 | 11.6 ± 0.385 |
| stearic | | C18:0 | 173 ± 40.2 | 196 ± 4.74 |
| oleic | (n-9) | C18:1 | 22.8 ± 3.43 | 1293 ± 70.4 |
| vaccenic | (n-7) | C18:1 | 1.99 ± 0.424 | 37.7 ± 0.892 |
| linoleic | | C18:2 | 642 ± 62.8 | 1081 ± 41.4 |
| linolenic | | C18:3 | 96.9 ± 17.9 | 166 ± 6.46 |
| arachidic | | C20:0 | 10.9 ± 2.08 | 12.2 ± 0.439 |
| arachidonic | | C20:4 | 2.13 ± 0.311 | 7.28 ± 0.288 |
| timnodonic | | C20:5 | 25.7 ± 4.57 | 46.6 ± 1.47 |
| adrenic | | C22:4 | 1.41 ± 0.312 | 1.69 ± 0.0711 |
| cervonic | | C22:6 | 4.12 ± 0.708 | 9.61 ± 0.354 |

High-fatted rats. Groups of male Wistar rats (n=21/diet) were ad libitum fed high-fat diets containing standard rat chow to which 40% (w/w) of lard, corn oil or olive oil had been added, from the time of weaning (age=21 d) until sacrifice at 50 d. Triacylglycerol from lard contains predominantly saturated fatty acids; that from corn oil predominantly monounsaturated fatty acids; and that from olive oil predominantly polyunsaturated fatty acids, see Table 4.

TABLE 4
FREE FATTY ACID COMPOSITIONS OF
FATS USED TO MAKE THE HIGH FAT DIET
Free fatty acid composition of lard, corn oil and olive oil.
Values are % of total

| Fatty Acid | Lard | Corn Oil | Olive Oil |
|---|---|---|---|
| C16:0 | 26.9 | 5.0 | 15.2 |
| C16:1 | 3.5 | — | 1.7 |
| C18:0 | 51.8 | 1.7 | 2.7 |
| C18:1 (n-9) | 3.5 | 10.7 | 66.7 |
| C18:1 (n-7) | — | 3.9 | 3.0 |
| C18:2 | 14.3 | 57.2 | 10.5 |
| C18:3 | — | 21.1 | — |

Soleus muscle tissue isolation and incuhation.Immersion and incubation media was Krebs-Henseleit buffer (KHB) containing 118.5 mM NaCl, 4.75 mM KCl, 1.18 mM MgSO₄.7H₂O and KH₂PO₄, 24.8 mM NaHCO₃, 2.54 mM CaCl₂ and 10 mM d-glucose. The medium was gassed continuously with 95% O_{2/5}% CO₂. Incubations were preformed in KHB alone or containing various concentrations of CGRP, amylin, norepinephrine, insulin or the CGRP and amylin antagonists where appropriate. Soleus muscles of both legs were excised keeping the tissue immersed in KHB; muscles were separated into two equal parts and transferred to the incubation flasks. At the completion of incubation the muscles were snap frozen in liquid nitrogen, and then trimmed of connective tissue and all visible fat. The muscle strips were stored at −80° C. until analysis.

Lipid extraction and chromatographic separation of fatty acids. Total lipids were extracted from muscle strips by a modified version of the method of Bligh and Dyer (Gorski et al., 1988, Mol & Cell Biochem 178:113-118; 1. Bligh et al., 1959, Can J Biochem Physiol 37: 911-917). Muscles were homogenised (Soniprep 150) in 0.4 ml of deionised water. Solvent volumes used herein were varied according to the water content of the muscle (ie muscle weight) as described (Bligh & Dyer, supra). 2 vols of methanol containing 0.005% butylated hydroxy toluene (BHT) and 1 vol chloroform were added to the muscle suspension. Homogenates were vortexed at maximum speed for 2 minutes and centrifuged at 2,500 g for 4 minutes. Supernatants were recovered and pellets re-extracted with 2 vol methanol, 1 vol chloroform and 0.8 vol 0.2 N HCl. Residues were vortexed for 2 min then centrifuged at 3,500 g for 3 min. After centrifugation the combined supernatants were diluted with 2v each of milli Q water and chloroform and the phases separated by centrifugation at 3,500 g for 4 minutes. The lower chloroform phase was recovered, neutralised by dropwise addition of 0.2 N methanolic NH₄OH and evaporated down in a stream of N₂. The samples were stored at −80° C. until required.

Aminopropyl phase (250 mg) was packed into 16 ml teflon columns with teflon frits placed at the top and bottom of the bonded phase. Columns were placed in a Vac Elut apparatus and washed twice with 2 ml portions of hexane (Prasad et al., 1988, J Chromato 428: 221-228; Kaluzny et al., 1985, J Lipid Res 26: 135-140). The dry lipid samples were taken up in two 0.150 ml portions of chloroform and applied to the column under atmospheric pressure. After adsorption the neutral lipids were eluted with 4 ml of chloroform-2-propanol (2:1, v/v) and the free fatty acids eluted with 4 ml of 2% acetic acid in diethyl ether. The solvent containing the free fatty acids was dried under a stream of N₂.

Gas-liquid chromatographic analysis. Dry free fatty acid residues were processed for derivatisation of methyl esters by the boron trifluoride-methanol method (Prasad, supra). 1 ml of boron trifluoride in methanol (BF3) was added to each sample, the sample vials were heated at 70° C. for 5 minutes, shaken vigorously, and baked for a further 10 minutes. Once the samples reached room temperature 0.5 ml milli Q water was added, the sample vials shaken, 0.1 ml heptane was added and the sample shaken again. 0.05 ml of the top heptane layer was removed for GLC analysis. A Model HP5890 Plus Series 2 (Hewlett-Packard) gas chromatograph equipped with a DB-225 column was used to separate the methyl esters of the fatty acids and a Model HP 5890 GC with a Model HP 5973 MS (Hewlett-Packard) GC/MS used to confirm the identity of individual free fatty acids. The temperature program consisted of a linear increase from an initial temperature of 80° C. to a final temperature of 210° C. at a rate of 3° C./min followed by a ten-minute period at the final temperature. The quantitation of tissue fatty acids was based on retention times of fatty acid methyl ester standards and relative theoretical response factors. Free fatty acids were assigned based on standards and on GC/MS chromatograms.

Determination of tissue free fatty acids and triacylglycerol concentration by enzymatic analyses. Free fatty acids were separated from total lipid, evaporated under a stream of N₂ and stored at −80° C. until analysis. Samples were dissolved in 50 μl of warm ethanol (35-40° C.), and 0.625 ml of a Triton X-100 (6% w/v) solution was added once the ethanol reached room temperature. The solution was stirred for 30 min, then made up to 0.825 ml with the Triton solution. Free fatty acids were quantified using a commercial method (Boehringer Mannheim) adapted for micro analyses (Cobas Mira, Roche Diagnostics), with palmitic acid standards. Triacyl glycerol levels were quantified in the total lipid fraction (Pointe Scientific) with a glycerol standard.

Enzymatic analysis of tissue glycerol. Frozen muscle samples were powdered and weighted into 2-ml eppendorf tubes. 0.5 ml of 10% (w/v) trichloroacetic acid was added to each sample, which was then homogenised for 4 minutes in ice. The homogenate was centrifuged and vortexed several times, rested on ice for 2 minutes then centrifuged at 10,000 rpm for 5 min. 0.4 ml of the supernatant was transferred to a new tube to which 0.5 ml of tri-n-octalamine/Freon 113 (1:2.03 v/v) was added. The solution was mixed and rested on ice for 5 minutes. The pH of the upper aqueous layer was determined and the sample mixed and centrifuged until a neutral pH was achieved. 0.2 ml of the upper layer was removed and analyzed for glycerol content.

Statistical analysis. All results are presented as means±S.E.M. To evaluate the effect of the hormones on lipid metabolites, a one-way analysis of the variance (ANOVA) was performed. Dunnett's post hoc test was applied to analyse differences between treatment and corresponding control values. All statistical analyses were performed using Macintosh Graph Pad Prism program, Power PC version. A value of P<0.05 was considered to be statistically significant.

### Example 2

### Effects of Amyin, CGRP, Norepinephrine and Insulin on Lipid Metabolites in Skeletal Muscle From Normally Fed Rats

This example describes the stimulation evoked by various agents on the levels of lipid metabolites in isolated rat soleus muscle tissue. Muscle tissue was treated with amylin, CGRP, norepinephrine, or insulin and the free fatty acids, glycerol, and triglyceride levels were compared with control levels.

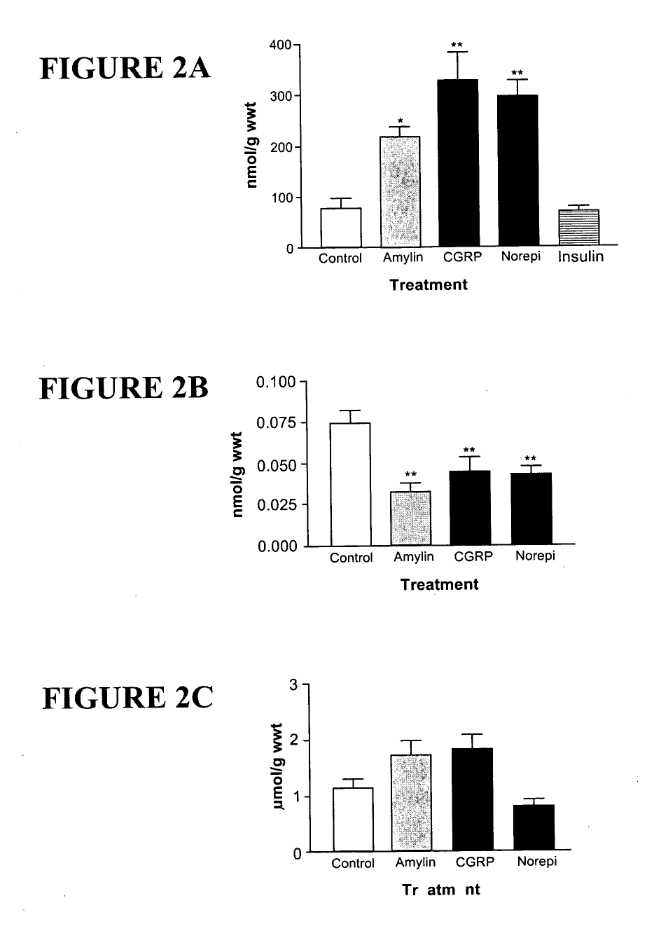

The effects of amylin, CGRP, insulin and norepinephrine on free fatty acid content in isolated soleus muscles are shown in FIG. 2A. There were significant differences in the free fatty acid content of soleus muscles treated with amylin, CGRP and norepinephrine compared to those in control muscle. Incubation with CGRP caused the greatest increase in intramuscular free fatty acids (325±56 nmol/g vs. 78±21, P<0.01), as compared with norepinephrine (295±27 nmol/g vs. 78±21, P<0.01) and amylin (216±22 nmol/g vs. 78±21, P<0.05). Incubation of soleus muscles with maximally stimulating insulin did not change the concentration of intramuscular free fatty acids compared to that in control tissue (67±9 nmol/g vs.78±21).

Amylin, CGRP and norepinephrine evoked decreases in the intramuscular glycerol content as seen in FIG. 2B. After 1 h incubation with amylin, the glycerol content was (0.03±0.004 nmol/g vs. 0.07±0.007, P<0.01), with CGRP (0.04±0.008 nmol/g vs. 0.07±0.007, P<0.01) and after norepinephrine treatment (0.04±0.005 nmol/g vs. 0.07±0.007, P<0.01).

FIG. 2C shows triacylglycerol content in soleus muscles after 60-min incubation. Treatment with norepinephrine lowered the triglyceride content (0.81±0.09 μmol/g vs. 1.17±0.15). There was also a tendency toward elevated muscle triglyceride with amylin and CGRP treatment (1.7±0.26 μmol/g (amylin), 1.9±0.23 (CGRP) vs. 1.17±0.15 (control)), but none of these effects were significantly different from with control values.

Incubation medium from all three hormone treatments, was tested using the same enzyme based methodologies (n=7 per treatment). No release of free fatty acids or glycerol into the medium was detected (results not shown).

### Example 3

### Effects of Hormones on the Content of Individual Free Fatty Acids in Skeletal Muscle

This example describes the quantitation of individual free fatty acids released in muscle tissue in response to treatment with CGRP, amylin, or norepinephrine. All procedures utilised gas chromatography to quantitate individual free fatty acids with confirmation of identity by mass spectrometry (GC-MS). Table 5 shows the fatty acid levels in all lipid fractions in resting soleus muscle as determined by analysis of total lipid samples. Total basal lipid samples were analyzed by gas chromatography, with confirmation of the identity of individual fatty acids by mass spectroscopy, to determine the resting fatty acid composition of soleus muscles. Values are means±S.E.M. (n=8 at each point)

TABLE 5
FATTY ACID COMPOSITION OF SOLEUS MUSCLE

| Fatty Acid | | | Concentration of FA (nmol/g wwt) |
|---|---|---|---|
| myristic | | C14:0 | 0.78 ± 0.45 |
| pentadecanoic | | C15:0 | 0.1 ± 0.033 |
| palmitic | | C16:0 | 8.05 ± 2.65 |
| palmitoleic | (n-9) | C16:1 | 0.11 ± 0.05 |
| palmitoleic | (n-7) | C16:1 | 0.1 ± 0.05 |
| margaric | | C17:0 | 0.133 ± 0.033 |
| stearic | | C18:0 | 4.25 ± 0.733 |
| oleic | (n-9) | C18:1 | 4.9 ± 1.95 |
| vaccenic | (n-7) | C18:1 | 1.25 ± 0.316 |
| linoleic | | C18:2 | 5.5 ± 1.55 |
| linolenic | | C18:3 | 0.25 ± 0.1 |
| arachidic | | C20:0 | 0.183 ± 0.13 |
| arachidonic | | C20:4 | 1.56 ± 0.316 |
| timnodonic | | C20:5 | 0.28 ± 0.083 |
| adrenic | | C22:4 | 0.083 ± 0.016 |
| cervonic | | C22:6 | 0.816 ± 0.166 |

Table 6 shows the hormone-evoked release of individual free fatty acids. This shows that C16:0 is the most abundant free fatty acid present in the soleus muscle, however, incubation in the presence of hormone did not cause an increase in concentration. The intramuscular concentration of C16:1 (n-9) was decreased on treatment with amylin (0.013±0.0011 nmol/g vs. 0.008±0.0013, P<0.05), CGRP (0.013±0.0011 nmol/g vs. 0.008±0.0006, P<0.01) and norepinephrine (0.013±0.0011 nmol/g vs. 0.007±0.0007, P<0.01).

The concentration of several of the long chain, poly-unsaturated free fatty acids increased by hormone treatment. Intramuscular arachidonic acid (C20:4) concentration was increased on treatment with amylin (0.005±0.001 nmol/g vs. 0.016±0.002, P<0.01), CGRP (0.005±0.001 nmol/g vs. 0.018±0.002, P<0.01) and norepinephrine (0.005±0.001 nmol/g vs. 0.015±0.0007, P<0.01). Cervonic acid concentration was also increased on treatment with all three hormones: amylin (0.01±0.0008 nmol/g vs. 0.03±0.004, P<0.01), CGRP (0.01±0.0008 nmol/g vs. 0.022±0.002, P<0.01) and norepinephrine (0.01±0.0008 nmol/g vs. 0.022±0.0015, P<0.05).

TABLE 6
HORMONE EVOKED RELEASE OF INDIVIDUAL FREE FATTY ACIDS
Hormone evoked release of individual free fatty acids, over 1 hour, in soleus muscle was analyzed by gas
chromatography, with confirmation the of identity of individual free fatty acids by mass spectroscopy.
Values are nmol/g wwt and are expressed as means ± S.E.M. (n = 16 for each point).

| Free fatty acid | Control | | Amylin | CGRP | Norepinephrine |
|---|---|---|---|---|---|
| myristic | C14:0 | 0.025 ± 0.002 | 0.026 ± 0.0028 | 0.028 ± 0.001 | 0.023 ± 0.001 |
| pentadecanoic | C15:0 | 0.01 ± 0.00005 | 0.013 ± 0.002 | 0.01 ± 001 | 0.01 ± 0.0012 |
| palmitic | C16:0 | 0.17 ± 0.74 | 0.21 ± 0.0188 | 0.23 ± 0.01* | 0.175 ± 0.011 |
| palmitoleic (n-9) | C16:1 | 0.013 ± 0.0011 | 0.008 ± 0.0013* | 0.008 ± 0.0006* | 0.007 ± 0.0007** |
| palmitoleic (n-7) | C16:1 | 0.013 ± 0.002 | 0.015 ± 0.0025 | 0.022 ± 0.002* | 0.015 ± 0.002 |
| margaric | C17:0 | 0.008 ± 0.0006 | 0.013 ± 0.0022* | 0.015 ± 0.0015* | 0.01 ± 0.0007 |
| stearic | C18:0 | 0.09 ± 0.0035 | 0.125 ± 0.0058** | 0.15 ± 0.01** | 0.1 ± 0.006 |
| oleic | C18:1 | 0.06 ± 0.0051 | 0.076 ± 0.0082 | 0.09 ± 0.0075** | 0.07 ± 0.0056 |
| vaccenic | C18:1 | 0.008 ± 0.0007 | 0.015 ± 0.0015** | 0.017 ± 0.0015** | 0.015 ± 0.0008** |
| linoleic | C18:2 | 0.035 ± 0.0045 | 0.058 ± 0.006** | 0.057 ± 0.006* | 0.043 ± 0.003 |
| linolenic | C18:3 | 0.001 ± 0.0007 | 0.01 ± 0.002* | 0.005 ± 0.0013 | 0.007 ± 0.0025 |
| arachidic | C20:0 | 0.007 ± 0.001 | 0.008 ± 0.002 | 0.007 ± 0.0008 | 0.003 ± 0.001 |
| arachidonic | C20:4 | 0.005 ± 0.001 | 0.016 ± 0.002** | 0.018 ± 0.002** | 0.015 ± 0.0007** |
| timnodonic | C20:5 | 0.002 ± 0.0007 | 0.005 ± 0.0007** | 0.003 ± 0.0005 | 0.002 ± 0.0005 |
| adrenic | C22:4 | 0.002 ± 0.001 | 0.008 ± 0.003* | 0.007 ± 0.002 | 0.007 ± 0.001 |
| cervonic | C22:6 | 0.01 ± 0.0008 | 0.03 ± 0.004** | 0.022 ± 0.002** | 0.022 ± 0.0015* |

*Significant difference compared with control (P < 0.05).
**Significant difference compared with control (P < 0.01).

Table 7 shows the percentage of intramuscular free fatty acid compared to total fatty acid levels. This Table also shows the change in the percentage of the free fatty acids available after hormone treatment.

TABLE 7
HORMONE EVOKED RELEASE OF FREE FATTY ACIDS
Hormone evoked release of free fatty acids, over 1 hour, in soleus
muscles was compared to total resting fatty acids. Values represent
percentage of free fatty acids released by hormone treatment
compared to total resting fatty acid levels.

| Fatty Acid | Control | Amylin | CGRP | Norepinephrine |
|---|---|---|---|---|
| C14:0 | 3.2 | 3.4 | 3.6 | 3.1 |
| C15:0 | 12.9 | 15.4 | 12.9 | 10.9 |
| C16:0 | 2.2 | 2.6 | 2.9 | 2.2 |
| C16:1 (n-9) | 10.6 | 7.2 | 7.5 | 6.0 |
| C16:1 (n-7) | 12.6 | 15.8 | 21.6 | 14.3 |
| C17:0 | 6.3 | 10.7 | 10.7 | 7.2 |
| C18:0 | 2.1 | 2.9 | 3.6 | 2.4 |
| C18:1 (n-9) | 1.2 | 1.6 | 1.8 | 1.4 |
| C18:1 (n-7) | 0.6 | 1.3 | 1.4 | 1.2 |
| C18:2 | 0.6 | 1.1 | 1.0 | 0.8 |
| C18:3 | 0.8 | 3.7 | 2.1 | 2.5 |
| C20:0 | 3.7 | 4.5 | 3.6 | 2.0 |
| C20:4 | 0.3 | 1.0 | 1.1 | 0.9 |
| C20:5 | 0.5 | 1.4 | 1.0 | 0.4 |
| C22:4 | 1.2 | 9.1 | 8.1 | 7.5 |
| C22:6 | 1.3 | 3.3 | 2.7 | 2.5 |

Table 3, supra, shows the levels of free fatty acids and total fatty acid levels present in the standard rat chow.

### Example 4

### Effects of Amylin, CGRP and Norepinephrine on the Soleus Muscle Triglyceride Content of Rats Fed High Fat Diets

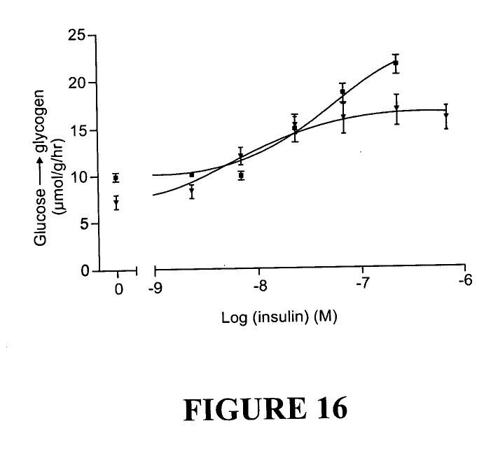

High fat fed rats are a model for insulin resistance, and are a especially useful model (compared to normal-fed animals) in the study of type 2 diabetes mellitus. Table 4, supra, shows the composition of the fats used to create the high fat diets. Tables 8, 9, & 10 show the individual free fatty acid content of soleus muscles from rats fed one of the three high fat diets. FIG. 16 shows that rats fed a high fat diet are insulin resistant, as determined by insulin response of the soleus muscle. Insulin dose response curves were measured in soleus muscle from rats fed a normal diet (′) or a high fat diet (□) for 51 days. Insulin responses were measured through incorporation of D[¹⁴C(U)] glucose into muscle glycogen following incubation for 2 h with various concentrations of insulin. Muscle glycogen was then extracted and analyzed for D[¹⁴C(U)] glucose content by liquid scintillation spectrometry. Dose-response curves were fitted to a sigmoidal dose-response algorithm. Results are shown as means±SEM (n=12-18 muscle strips for each point). Sum of squares analysis revealed highly significant differences between the two curves (P<10⁻⁵).

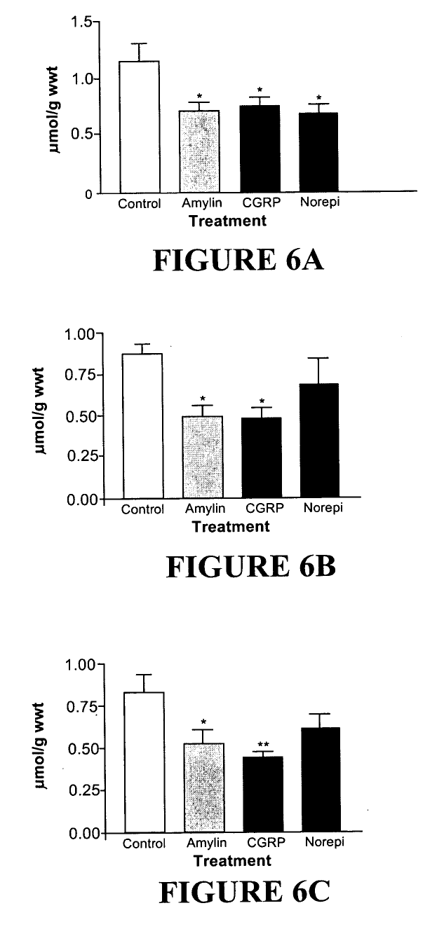

FIG. 6 shows the effects of the three hormones on the triglyceride content in soleus muscle of rats fed one of three high fat diets. In the group fed a diet containing 40% added lard (FIG. 6A), all three hormones caused significant decrements in soleus muscle triglyceride content compared to the corresponding control (0.75±0.08 (CGRP), 0.71±0.07 (amylin) and 0.69±0.09 (norepi) μmol/g vs. 1.14±0.16 μmol/g, P<0.05 each case). In the groups fed with 40% corn oil (FIG. 6B) (0.48±0.06 (CGRP) and 0.49±0.07 (amylin) [mol/g vs. 0.89±0.06 μmol/g, P<0.05 in each case) or 40% olive oil (FIG. 6C) (0.52±0.08 (CGRP) and 0.44±0.4 (amylin) μmol/g vs. 0.83±0.1 μmol/g, P<0.05 in each case), only amylin and CGRP caused significant decreases compared to control.

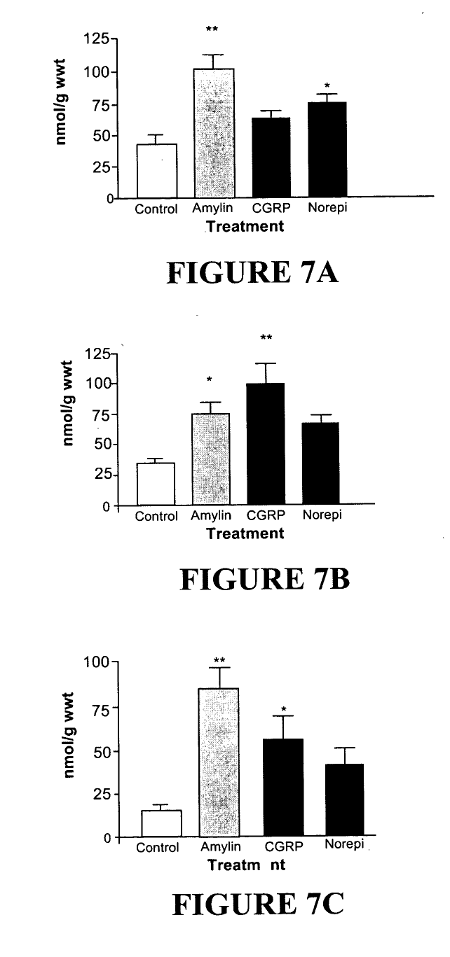

FIG. 7 shows the effects of the three hormones on the free fatty acid content in the soleus muscle of rats fed one of three high fat diets. In the groups fed a diet containing 40% added lard (FIG. 7A), amylin and norepinephrine caused significant increases in soleus muscle free fatty acid content compared to the corresponding control (102±12 (amylin, P<0.01), 75±7 (norepi, P<0.05) nmol/g vs. 43±8). CGRP a tendency to evoke an increase in the free fatty acid content this was not significantly different to control values (64±7 nmol/g vs. 43±8). In the groups fed with 40% cornoil (FIG. 7B) (75±9 (amylin, P<0.05) and 99±17 (CGRP, P<0.01) nmol/g vs. 35±4 nmol/g) or 40% olive oil (FIG. 7C) (85±12 (amylin, P<0.01) and 55±14 (CGRP, P<0.05) nmol/g vs. 15±3 nmol/g), both CGRP and amylin caused significant increases in soleus muscle free fatty acid content.

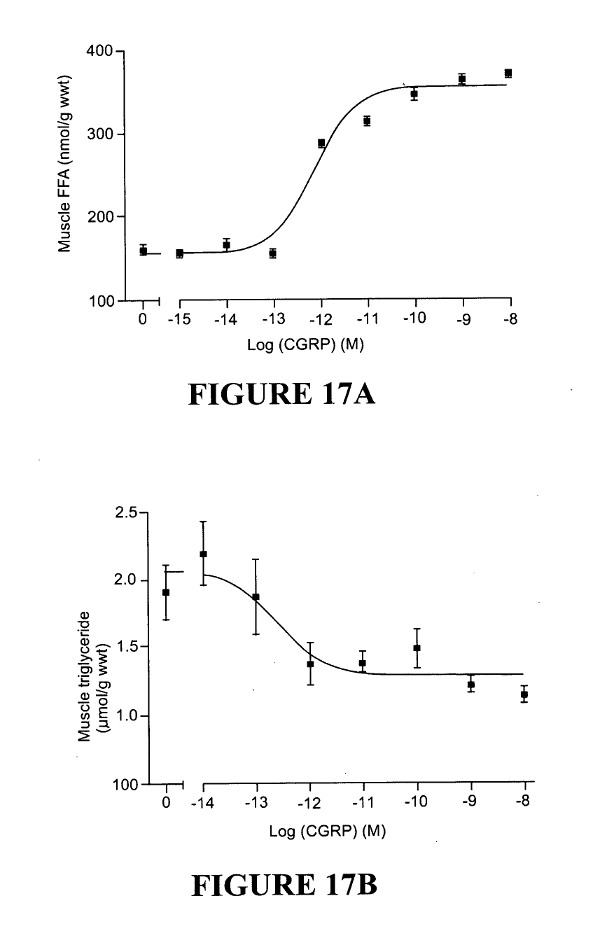

Both the triglyceride and free fatty acid effects of CGRP-1 administration to high fat-fed animals occur via the high affinity CGRP1 site, as demonstrated by the similar EC₅₀ dose-response values. FIG. 17A shows CGRP-1 evoked a dose-dependent increase in muscle NEFA content with an EC₅₀ similar to the high affinity site observed in muscle from normal fed animals (0.7 pM±0.4 to 1.0; mean±95% C.I.). FIG. 17B shows that, associated with the increase in NEFA content was a dose-dependent decrease in total intramuscular triglyceride content, an effect not observed in soleus from normal-fed animals. The EC₅₀ for the decrease in intramuscular triglyceride (0.25 pM±0.03 to 1.96; mean±95% C.I.) was identical within experimental error to the observed EC₅₀ for the increase in NEFA content. Basal triglyceride content in muscle from high fat-fed animals was significantly elevated compared to muscle from normal-fed animals. In this experiment, the animals were fed a diet high in saturated fat (lard) for 30 days from the time of weaning.

TABLE 8
HORMONE EVOKED RELEASE OF INDIVIDUAL FREE FATTY ACIDS
IN SOLEUS MUSCLES OF RATS FED A DIET HIGH IN CORN OIL
Hormone evoked release of individual free fatty acids, over 1 hour, in soleus muscle of rats fed a diet
high in corn oil was analyzed by gas chromatography, with confirmation the of identity of individual free
fatty acids by mass spectroscopy. Values are nmol/g wwt and are expressed as means ± S.E.M. (n = 16
for each point).

| Free fatty acid | | Control | Amylin | CGRP | Norepinephrine |
|---|---|---|---|---|---|
| myristic | C14:0 | 0.02 ± 0.001 | 0.04 ± 0.006** | 0.03 ± 0.02** | 0.02 ± 0.002 |
| pentadecanoic | C15:0 | 0.01 ± 0.001 | 0.05 ± 0.007** | 0.04 ± 003** | 0.04 ± 0.005** |
| palmitic | C16:0 | 0.07 ± 0.006 | 0.12 ± 0.01 | 0.16 ± 0.03** | 0.09 ± 0.01 |
| palmitoleic (n-9) | C16:1 | 0.12 ± 0.007 | 0.19 ± 0.008** | 0.20 ± 0.008** | 0.15 ± 0.01 |
| palmitoleic (n-7) | C16:1 | 0.005 ± 0.0005 | 0.01 ± 0.0008** | 0.01 ± 0.0008** | 0.007 ± 0.0005 |
| margaric | C17:0 | 0.03 ± 0.006 | 0.02 ± 0.0009 | 0.02 ± 0.003 | 0.04 ± 0.006 |
| stearic | C18:0 | 0.09 ± 0.004 | 0.14 ± 0.008 | 0.39 ± 0.02 | 0.11 ± 0.008 |
| oleic | C18:1 | 0.08 ± 0.008 | 0.13 ± 0.01* | 0.16 ± 0.01** | 0.12 ± 0.02 |
| vaccenic | C18:1 | 0.02 ± 0.002 | 0.02 ± 0.002 | 0.02 ± 0.004 | 0.02 ± 0.001 |
| linoleic | C18:2 | 0.08 ± 0.005 | 0.18 ± 0.02** | 0.18 ± 0.008** | 0.14 ± 0.02* |
| linolenic | C18:3 | 0.07 ± 0.005 | 0.16 ± 0.02** | 0.17 ± 0.01** | 0.13 ± 0.01** |
| arachidic | C20:0 | 0.03 ± 0.003 | 0.04 ± 0.002 | 0.03 ± 0.005 | 0.03 ± 0.002 |
| arachidonic | C20:4 | 0.02 ± 0.002 | 0.04 ± 0.002 | 0.02 ± 0.002 | 0.08 ± 0.009** |
| timnodonic | C20:5 | 0.02 ± 0.002 | 0.05 ± 0.007** | 0.04 ± 0.001** | 0.05 ± 0.005** |
| adrenic | C22:4 | 0.02 ± 0.002 | 0.03 ± 0.003 | 0.04 ± 0.002** | 0.05 ± 0.004** |
| cervonic | C22:6 | 0.01 ± 0.0007 | 0.03 ± 0.002** | 0.03 ± 0.003** | 0.04 ± 0.003** |

*Significant difference compared with control (P < 0.05).
**Significant difference compared with control (P < 0.01).

TABLE 9
HORMONE EVOKED RELEASE OF INDIVIDUAL FREE FATTY ACIDS
IN SOLEUS MUSCLES OF RATS FED A DIET HIGH IN OLIVE OIL
Hormone evoked release of individual free fatty acids, over 1 hour, in soleus muscle of rats fed a diet
high in olive oil was analyzed by gas chromatography, with confirmation the of identity of individual
free fatty acids by mass spectroscopy. Values are nmol/g wwt and are expressed as means ± S.E.M.
(n = 16 for each point).

| Free fatty acid | | Control | Amylin | CGRP | Norepinephrine |
|---|---|---|---|---|---|
| myristic | C14:0 | 0.004 ± 0.0003 | 0.01 ± 0.001** | 0.015 ± 0.0009** | 0.005 ± 0.0002 |
| pentadecanoic | C15:0 | 0.009 ± 0.0020 | 0.04 ± 0.005** | 0.08 ± 002** | 0.01 ± 0.0004 |
| palmitic | C16:0 | 0.07 ± 0.02 | 0.18 ± 0.01 | 0.20 ± 0.006** | 0.19 ± 0.002* |
| palmitoleic (n-9) | C16:1 | 0.16 ± 0.03 | 0.29 ± 0.03** | 0.27 ± 0.013* | 0.23 ± 0.01 |
| palmitoleic (n-7) | C16:1 | 0.01 ± 0.0006 | 0.02 ± 0.001 | 0.02 ± 0.002 | 0.03 ± 0.009* |
| margaric | C17:0 | 0.009 ± 0.002 | 0.02 ± 0.003 | 0.03 ± 0.012 | 0.01 ± 0.001 |
| stearic | C18:0 | 0.09 ± 0.01 | 0.18 ± 0.02** | 0.12 ± 0.004 | 0.16 ± 0.006** |
| oleic | C18:1 | 0.10 ± 0.01 | 0.34 ± 0.04** | 0.34 ± 0.01** | 0.28 ± 0.03** |
| vaccenic | C18:1 | 0.06 ± 0.01 | 0.13 ± 0.02** | 0.17 ± 0.008** | 0.2 ± 0.003 |
| linoleic | C18:2 | 0.007 ± 0.0006 | 0.02 ± 0.004 | 0.06 ± 0.01** | 0.04 ± 0.009* |
| linolenic | C18:3 | 0.009 ± 0.001 | 0.01 ± 0.002 | 0.03 ± 0.0009 | 0.04 ± 0.01** |
| arachidic | C20:0 | 0.01 ± 0.0009 | 0.04 ± 0.005** | 0.04 ± 0.001** | 0.009 ± 0.0004 |
| arachidonic | C20:4 | 0.01 ± 0.002 | 0.03 ± 0.007** | 0.02 ± 0.007 | 0.03 ± 0.0008 |
| timnodonic | C20:5 | 0.009 ± 0.002 | 0.02 ± 0.003 | 0.01 ± 0.001 | 0.04 ± 0.004** |
| adrenic | C22:4 | 0.01 ± 0.002 | 0.05 ± 0.008** | 0.03 ± 0.006* | 0.02 ± 0.0007 |
| cervonic | C22:6 | 0.02 ± 0.003 | 0.08 ± 0.02 | 0.11 ± 0.03* | 0.12 ± 0.03* |

*Significant difference compared with control (P < 0.05).
**Significant difference compared with control (P < 0.01).

TABLE 10
HORMONE EVOKED RELEASE OF INDIVIDUAL FREE FATTY ACIDS
IN SOLEUS MUSCLES OF RATS FED A DIET HIGH IN LARD
Hormone evoked release of individual free fatty acids, over 1 hour, in soleus muscle of rats fed a
diet high in lard was analysed by gas chromatography, with confirmation the of identity of individual
free fatty acids by mass spectroscopy. Values are nmol/g wwt and are expressed as means ± S.E.M.
(n = 16 for each point).

| Free fatty acid | | Control | Amylin | CGRP | Norepinephrine |
|---|---|---|---|---|---|
| myristic | C14:0 | 0.004 ± 0.0007 | 0.05 ± 0.01** | 0.03 ± 0.007* | 0.04 ± 0.007** |
| pentadecanoic | C15:0 | 0.04 ± 0.01 | 0.12 ± 0.03* | 0.14 ± 0.01** | 0.13 ± 0.02* |
| palmitic | C16:0 | 0.1 ± 0.009 | 0.24 ± 0.01** | 0.24 ± 0.01** | 0.23 ± 0.02** |
| palmitoleic (n-9) | C16:1 | 0.02 ± 0.001 | 0.02 ± 0.003 | 0.03 ± 0.003* | 0.01 ± 0.001 |
| palmitoleic (n-7) | C16:1 | 0.03 ± 0.005 | 0.02 ± 0.0009 | 0.02 ± 0.005 | 0.02 ± 0.0008 |
| margaric | C17:0 | 0.03 ± 0.005 | 0.08 ± 0.01** | 0.09 ± 0.01** | 0.08 ± 0.008* |
| stearic | C18:0 | 0.2 ± 0.03 | 0.35 ± 0.04* | 0.35 ± 0.06* | 0.22 ± 0.01 |
| oleic | C18:1 | 0.17 ± 0.02 | 0.2 ± 0.01 | 0.25 ± 0.02* | 0.21 ± 0.01 |
| vaccenic | C18:1 | 0.01 ± 0.002 | 0.02 ± 0.002 | 0.03 ± 0.006* | 0.07 ± 0.003** |
| linoleic | C18:2 | 0.12 ± 0.009 | 0.29 ± 0.004** | 0.23 ± 0.005* | 0.09 ± 0.003 |
| arachidic | C20:0 | 0.02 ± 0.001 | 0.06 ± 0.007** | 0.07 ± 0.004** | 0.03 ± 0.004 |
| arachidonic | C20:4 | 0.01 ± 0.003 | 0.01 ± 0.0005 | 0.02 ± 0.001 | 0.012 ± 0.003 |
| timnodonic | C20:5 | 0.01 ± 0.002 | 0.02 ± 0.002 | 0.02 ± 0.002 | 0.02 ± 0.004 |
| adrenic | C22:4 | 0.02 ± 0.003 | 0.03 ± 0.005* | 0.04 ± 0.005* | 0.02 ± 0.003 |
| cervonic | C22:6 | 0.01 ± 0.002 | 0.02 ± 0.002 | 0.015 ± 0.002 | 0.01 ± 0.001 |

*Significant difference compared with control (P < 0.05).
**Significant difference compared with control (P < 0.01).

### Example 5

### Effect of Amylin-(8-37) and CGRP-(8-37) on Hormone-mediated Modulation of Lipid Metabolism in Skeletal Muscle From Normal-fed and High-fat Fed Rats

A. Effects of Amylin-(8-37) and CGRP-(8-37) on Hormone-Mediated Modulation of Lipid Metabolism in Skeletal Muscle From Normal-Fed Rats

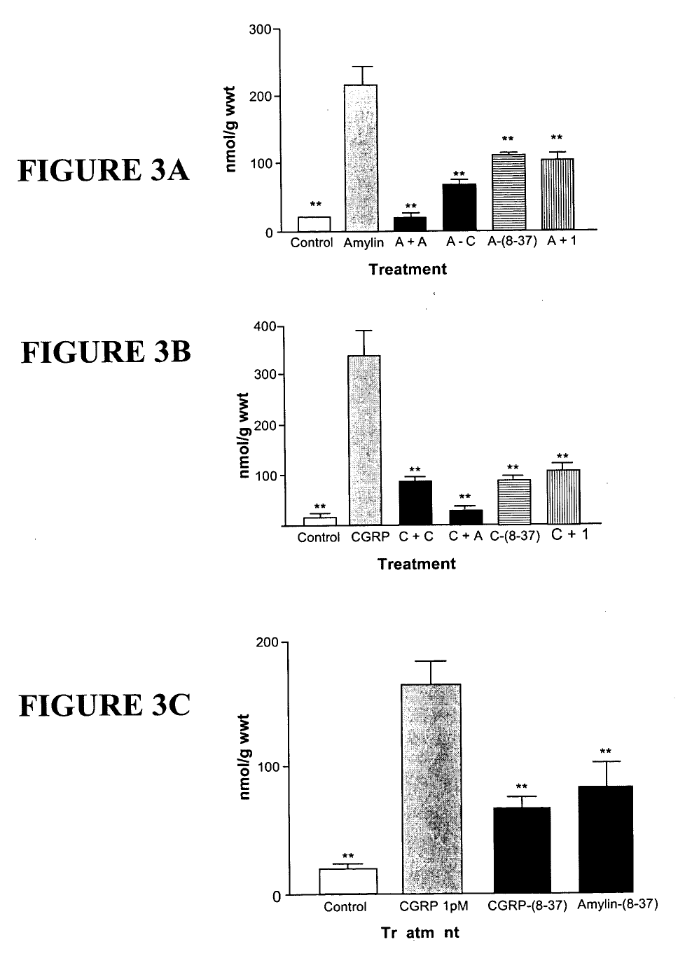

An in vitro study in isolated soleus muscle strips, based on enzymatic analysis, demonstrated that 10 μM amylin-(8-37) or 10 μM CGRP-(8-37) when used in conjunction with 100 nM amylin substantially decreased the intramuscular content of free fatty acids with compared to incubation with 100 nM amylin alone (18.5±6 nmol/g vs. 216±26, P<0.01) and (65±8 nmol/g vs. 216±6, P<0.01) respectively, see FIG. 3A. These linear antagonistic peptides both lack the first seven amino acids, and therefore an homologous NH₂-terminal cys2-cys7 disulfide ring structure shared by the full-length peptides from which they were derived. Both reverse a number of biological effects evoked by CGRP and amylin, and compete with varying effectiveness against corresponding radiolabeled analogues in several different tissues (Wang et al., 1991, FEBS Lett. 291:195-198; Chantry et al., 1991, Biochem. J. 277:139-143; Beaumont et al., 1998, Am. J. Physiol. 274:F51-62; Aiyar et al., 1995, J. Neurochem. 65:1131-1138; Bhogal et al., 1994, Peptides 15:1383-1390; Perry et al., 1997, Endocrinology 138:3486-3496; Zimmermann et al., 1997, J. Endocrinol. 155:423-431). Amylin-(8-37) suppressed the ability of CGRP-100 nM to increase soleus muscle free fatty acid content compared to incubation with 100 nM CGRP alone (34±5 nmol/g vs. 334±52, P<0.01), see FIG. 3B. The CGRP antagonist, CGRP-(8-37), also reversed the increase of free fatty acids evoked by 100 nM CGRP (90±7 nmol/g vs. 334±52, P<0.01). Addition of maximally stimulating insulin suppressed the ability of 100 nM amylin (216±26 nmol/g vs. 100±11, P<0.01) or CGRP (334±52 nmol/g vs. 107±17, P<0.01) to evoke an increase in the soleus free fatty acid content. Thus, insulin antagonizes the effects of both these hormones to elicit release of free fatty acids in skeletal muscle.

At a CGRP concentration of 1 pM, 100 pM CGRP-(8-37) and amylin-(8-37) can significantly reduce the soleus muscle free fatty acid concentration (68±8 nmol/g vs. 166±18, P<0.01) and (86±20 nmol/g vs. 166±18, P<0.01) respectively, see FIG. 3C.

This study provides direct evidence that both ^{8,37}amylin and ^{8,37}CGRP can inhibit the rise in intramuscular free fatty acids evoked by amylin and CGRP, i.e., by acting as competitive antagonists at a constituted CGRP/amylin receptor. The amylin-derived antagonist, ^{8,37}amylin, exhibited a significantly higher K, for the constituted receptor than did the corresponding CGRP-derived antagonist, 8,37CGRP. The approximately 20-fold difference in binding affinities is quantitatively consistent with the more effective blockade by ^{8,37}CGRP of amylin's action on glucose uptake. The demonstrated reversal, by both 1 μM and 10 μM ^{8,37}CGRP, of 10 nM amylin-mediated inhibition of insulin-stimulated glucose uptake, is consistent with the observed binding affinities of these peptides for the constituted C1ₘₛ/ramp 1 receptor. Thus, binding affinities of the antagonists, ^{8,37}CGRP and ^{8,37}amylin measured for the C1ᵢₙₛ₋/ramp 1 receptor in L6 myoblasts, are consistent with their relative potency in isolated soleus muscle. Similarly, cross-reactivity of ^{8,37}CGRP is consistent with the ability of this antagonist to completely reverse amylin-inhibition of insulin-stimulated glycogen synthesis and amylin-induced increases in blood glucose and lactate (Wang et al., 1991, FEBS Lett. 291:195-198). This order of antagonist potency, antagonist order, is also consistent with the hypothesis that CGRP and amylin elicit effects on glucose metabolism in skeletal muscle via binding to an endogenous C1ᵢₙₛ₋/ramp 1 receptor.

B. Receptor Antagonists of CGRP Reverse CGRP-1 Effects on Muscle Lipid in High Fat-Fed Rats

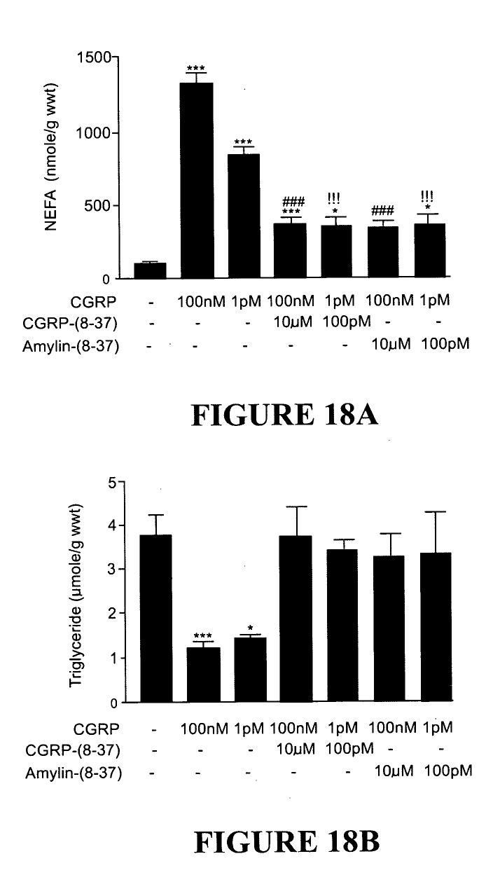

Soleus muscles from Wistar rats fed a high fat diet (40% lard) from the age of weaning (21 days) for 30 days (until 51 days) were excised and incubated for 1 h in vitro with CGRP1 in the absence or presence of the CGRP antagonists, amylin-(8-37) or CGRP-(8-37). Total lipid was extracted and quantitated. Results are shown in FIGS. 18A NEFA and 18B triglyceride.

As in normal fed rats, the CGRP receptor antagonists reversed the effects of CGRP. The decrease in triglyceride content evoked by either 1 pM or 100 nM CGRP1 was inhibited by a 10-fold molar excess of either antagonist. For the high affinity site, a 100 pM concentration of antagonist was sufficient to ameliorate the effects of 1 pM CGRP on soleus muscle triglyceride and free fatty acid contents.

### Example 6

### Dose-dependent Effects of Amylin to Stimulate Free Fatty Acid Content in Incubated Rat Soleus Muscle

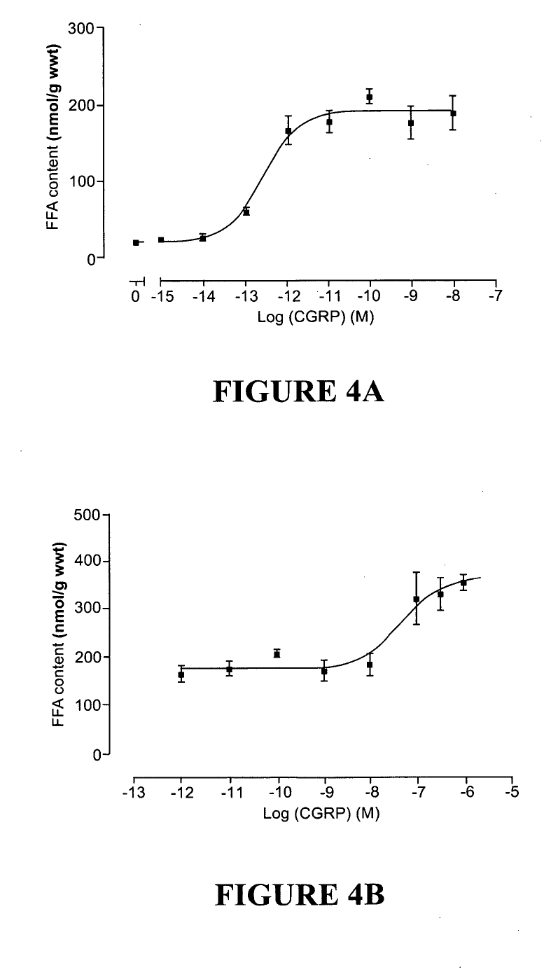

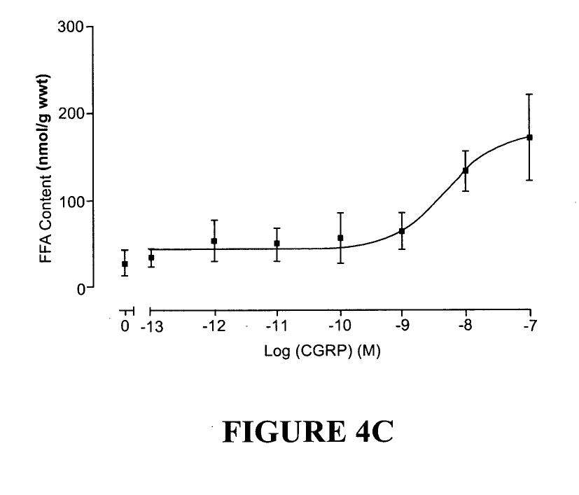

Dose-dependlent effects of CGRP to stimulate free fatty acid content in incubated rat soleus muscle. Data from CGRP-1 dose-response studies are shown in FIGS. 4A and 4B. These studies revealed that there are two concentrations ranges at which CGRP elicits increased free fatty acid content in incubated rat soleus muscle. The EC₅₀ value for the first phase is 0.25 pM±(13-0.5 pM [95% confidence interval]) with a corresponding r² value of 0.84. The EC₅₀ value for the second phase is 45 nM±(8.5 to 95) with corresponding r² value of 0.51. Data from the CGRP-2 dose-response study is shown in FIG. 4C. This study demonstrates that CGRP-2 has a different mechanism of action than its counterpart, is in fact similar to amylin. The EC₅₀ value is 5.2 nM±(2.3 to 11.5; mean±95% C.I.) with a corresponding r² value of 0.78.

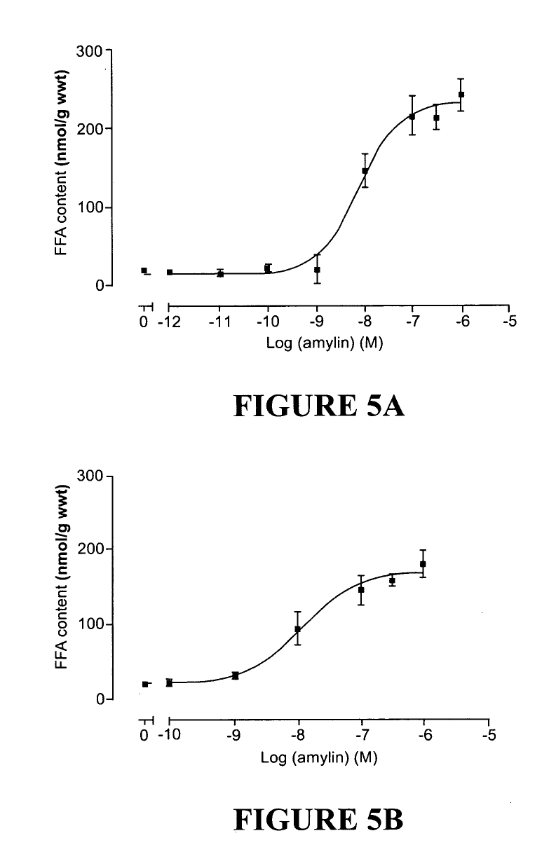

Dose-dependent effects of amylin to stimulate free fatty acid content in incubated rat soleus muscle. Dose-dependent effects of amylin to stimulate free fatty acid content in incubated rat muscle are illustrated in FIG. 5A. Progressive increases in amylin concentration in the incubation media increased the free fatty acid content in rat soleus muscle. The EC₅₀ for this effect was 7.8 nM±(4.1 to 14.7; mean±95% C.I.), with a corresponding r² value of 0.87.

Dose-dependent effects of amylin to stimulate free fatty acid content in incubated rat soleus muscle in the presence of CGRP (100 pM).

FIG. 5B shows the amylin dose response curve obtained in the presence of 100 pM CGRP. The EC₅₀ value is 11.6 nM±(4.8 to 27; mean±95% C.I.), with corresponding r² value of 0.79.

Calculated metabolic energy available from free fatty acids in skeletal muscle following hormone treatment. Table 11 shows the potential metabolic energy available from free fatty acids released following 60-min treatment of soleus muscles with amylin, CGRP and norepinephrine. Total levels were obtained from GC data. The amount of potential energy available from individual free fatty acids was calculated based on potential ATP production from complete mitochondrial fatty acid β-oxidation (Mathews et al., 2000, BIOCHEMISTRY, 3RD ED., Benjamin Cummings Pub. Co., California.). These calculations assume an ideal energy conversion of 100%. Increased intramuscular content of free fatty acids evoked by amylin and CGRP allow for significantly greater potential energy compared to control (2.73±0.28 μmol/g vs. 1.97±0.31, P<0.01), (2.96±0.22 μmol/g vs. 1.97±0.31, P<0.01). Potential metabolic energy available from free fatty acids after norepinephrine treatment was not significantly different to control (2.24±0.17 μmol/g).

TABLE 11
METABOLIC ENERGY POTENTIALLY AVAILABLE
FROM FREE FATTY ACIDS FOLLOWING HORMONE
TREATMENT
Total hormone evoked release of free fatty acids, over 1 hour, in
soleus muscles based on gas chromatography, with confirmation of
the identity of individual free fatty acids by mass spectroscopy.
Amounts of free fatty acids were used to calculate the theoretical
metabolic energy available from complete conversion of free fatty
acids to energy via mitochondrial fatty acid β-oxidation. These
calculations assume an energy conversion efficiency of 100%. Values
are means ± S.E.M (n = 16 at each point). Hormone concentrations
are 100 nM for all.

| | Control | Amylin | CGRP | Norepinephrine |
|---|---|---|---|---|
| Total | 0.467 ± 0.03 | 0.636 ± 0.05** | 0.691 ± 0.04** | 0.527 ± 0.03 |
| free | | | | |
| fatty | | | | |
| acids | | | | |
| (nmol/g | | | | |
| wwt) | | | | |
| Energy | 63.56 ± 5.2 | 88.2 ± 9.08 | 95.55 ± 7.28 | 72.58 ± 5.58 |
| avail- | | | | |
| able | | | | |
| (μmol | | | | |
| ATP/g/ | | | | |
| hr) | | | | |
| (kJ/g/ | 1.97 ± 0.31 | 2.73 ± 0.28** | 2.96 ± 0.22** | 2.24 ± 0.17 |
| hr) | | | | |

**Significant difference compared with control (P < 0.01)

### Example 7

### Materials and Methods

This example describes the materials and methods used in the experiments described in Examples 8-14, including preparation of cells expressing a recombinant receptor.

Peptides, radiochemicals and other analytical reagents. Rat amylin, whose sequence is identical to that of murine amylin (Cooper et al., 1994, Endocr. Rev. 15:163-201, 1994), and is hereinafter designated ‘amylin’ (lot 0538912); rat CGRP-1 (‘CGRP’; lot 501460); rat calcitonin (lot ZJ375); salmon calcitonin (‘sCT’; lot ZM423); ^{8,37}rat amylin (lot 515439); and ^{8,37}rat CGRP-1 (lot 501395) were purchased from Bachem (Switzerland). Human adrenomedullin was synthesised as previously described (Heller et al., 2000, Anal. Biochem. 285:100-104). Integrity and purity of all peptide preparations were confirmed by mass spectrometry (matrix-assisted laser-desorption ionisation time-of-flight MS, MALDI-TOF; Hewlett-Packard G2025A; 4-OH-cinnamic acid matrix). Soluble human insulin was employed throughout as the insulin agonist (‘insulin’; Actrapid 100U, Novo Nordisk). Bulk synthetic peptides were stored as powders desiccated at −80° C. under argon until use. They were dissolved in 18 MΩ doubly deionized water to yield stock solutions, which were added to reaction media as required to achieve stated final concentrations. [2-³H]2-deoxy-D-glucose ([3H]2-DOG; lot 2711-158, 30.6 Ci/mmol) and D[¹⁴C(U)]glucose (lot 2643-217, 10.6 mCi/mmol) were from New England Nuclear; D[¹⁴C(U)]sorbitol was from American Radiolabeled Chemicals Inc. (lot no 950314, 320 mCi/mmol); and [α-³²P]dCTP was from Amersham (lot AO 256 94; 3000 Ci/mmol). All other reagents were of analytical grade unless otherwise specified.

Synthesis and analysis of radiolabelled hormone analogs. CGRP, amylin, and sCT were radiolabeled by reductive methylation with [³H]NaBH₄ then purified as described previously (Cornish et al., 1997, Am. J. Phys. 36:E 1113-E1120). ³H-labeled peptides were purified by gel filtration followed by RP-HPLC, and incorporation of the [³H]methyl label confirmed by MALDITOF-MS. Measured specific activities for [³H]radioligands were: CGRP, 26.2 GBq/mmol; amylin, 24.0 GBq/mmol; and sCT, 23.2 GBq/mmol. Radioligands were stable for >12 months when stored desiccated under argon at −80° C.

Cloning of murine ramp 1, murine ramp 3, and the insert-negative form of the murine calcitonin receptor (C1ᵢₙₛ₋). Molecular biology protocols were as described (Sambrook J., 1989, Molecular cloning: a laboratory manual, Cold Spring Harbour Laboratory Press). Ramp 1 and ramp 3 (genbank accession numbers AA544629 and AF146524, respectively) were cloned by RT-PCR from total RNA (1 μg) extracted from murine soleus muscle. This was heated at 65° C. for 10 min and primed using random hexamers. Reverse transcription was performed using Expand™-RT for 10 min at 30° C., followed by 45 min at 42° C. Reaction conditions were: tris-HCl (50 mM); KCl (40 mM); MgCl₂ (5 mM); Tween-20 (0.5%); dithiothreitol (10 mM); RNase inhibitor (20 U); Expand™ reverse transcriptase (50 U) (Boehringer Mannheim); dNTP's (1 mM); pH 8.3. Oligonucleotide primers for ramp 1 and ramp 3 respectively were 5-TTAGGATCCGTTGCCATGGCCCGGCTGCGGCTCCCG-3 (sense)/5-CGAGAAT TCCTCATCACCCGGGATACCTA-3 (antisense) and 5-ATAGGATCCTG CATCTTAGTTGGCCATGA-3 (sense)/5-ATAGAATTCATCCAGCAGATCCTCA AGC-3 (antisense). Underlined nucleotides respectively indicate BamHI and EcoRI restriction sites introduced to facilitate cloning. PCR amplification was performed in an Eppendorf Mastercycler R gradient Thermal cycler for 40 cycles of 94° C. (1 min), 55° C. (2 min), and 72° C. (3 min). Reaction conditions were: Tris/HCl (10 mM), KCl (50 mM), MgCl₂ (1.1 mM), oligonucleotides (0.5 μM), gelatin (0.01% w/v), dNTP's (200 μM), 2.5 U REDTaq™ DNA polymerase (Sigma, Saint Louis), cDNA template (10 ng), at pH 8.3. PCR products were isolated and subcloned into pcDNA3.1+(Invitrogen) through the BamHI and EcoRI restriction sites. Sequence analysis confirmed the fidelity of sequences for both ramp 1 and ramp 3.

The insert-negative form of the calcitonin receptor, C1ᵢₙₛ₋ (Genbank accession U18542) was cloned from murine brain total RNA by RT-PCR using oligonucleotide primers 5-CCATCCCTGCCTGCAGATGC-3 (sense) and 5-CTCTATTTCTAGACTGACTCCC-3 (antisense); underlined residues refer to PstI and XbaI restriction sites introduced to facilitate cloning, while the bolded residue denotes a point mutation that introduced a restriction site. RT-PCR was carried out using Elongase™. Amplification was performed at 94° C. (30 s), 56° C. (30 s), 68° C. (2 min) for 5 cycles, then at 94° C. (30 s), 60° C. (30 s), 68° C. (2 min) for 30 cycles. A 1.9 kbp PCR product corresponding to calcitonin receptor C1ᵢₙₛ₋ was subcloned into pGEM-T (Promega) then re-cut with EcoRI and XbaI. The resulting two fragments derived from C1ᵢₙₛ₋ were then subcloned into pcDNA3.1 through a double cloning step via the EcoRI and XbaI sites. The fidelity of the murine C1ᵢₙₛ₋ sequence was confirmed by sequence analysis. We also employed RT-PCR cloning with sequencing of the cloned product to show that RNA corresponding to C1ᵢₙₛ₋ is expressed in rat soleus muscle, where it was the major RNA species corresponded to calcr detected (results not shown).

Transfection of L6 myoblasts and cos-7 cells, and analysis of radioligand binding. L6 myoblasts and cos-7 cells were obtained from ATCC. Both cell lines were cultered in Dulbecco's modified Eagle's medium (DMEM; Gibco/BRL, high glucose) supplemented with 10% fetal calf serum (Gibco/BRL, Gaithersburg, Md.), L-glutamine (2 mM), penicillin (100 U/ml; Sigma), streptomycin (100 mg/ml; Sigma), saline (0.85%), under 5% CO₂/95% air at 37° C. The day before transfection, cells were trypsinised and seeded into six-well plates at a density of 2.9×10⁵ cells/well in the same medium. For transfection, cells were incubated with a mixture comprising 1 μg each of Qiagen-purified (Qiagen, Valencia, Calif.) plasmids containing C1ᵢₙₛ₋ with either ramp 1 or ramp 3, and Fugene reagent (6 μl; Boehringer Mannheim) in Opti-Mem I medium (Gibco/BRL). The complex was added with serum to cells, which were incubated for a further 48 h.

For binding experiments, radioligand (either alone or together with stated final concentrations of non-radiolabeled ligands) was dissolved in DMEM containing 0.1% BSA (binding buffer) for 1 h before binding experiments were performed. Binding conditions were selected on the basis of previous time-course experiments (data not shown). Cells were washed twice with this buffer, then incubated with 1 ml of the same plus radioligand for 4 h at room temperature (21° C.). Next, cells were washed twice with ice-cold binding buffer, and cell-bound radioactivity counted by liquid scintillation spectrometry following transfer of cells with two washes of 0.5 M sodium hydroxide.

Northern blot analysis. Total RNA was isolated using Trizol™ Reagent (Life Technologies) according to manufacturer's instructions. Total RNA (5 μg/lane) was electrophoresed on a formaldehyde/agarose gel (1% w/v), transferred to a Nylon Hybond™ membrane (Amersham Pharmacia Biotech) then cross-linked with a Stratalinker 2400 (Stratagene, La Jolla, Calif.). Mouse ramp 1 and ramp 3 cDNA hybridisation probes were random primed with [α-³²P]dCTP. Filters were pre-hybridised for 2 h at 65° C. in Church-Gilbert solution (0.25 M disodium phosphate, 7% SDS, 1 mM EDTA), then hybridisation performed overnight at 65° C. in the same buffer containing 25 ng/ml of labelled probe. Filters were washed twice for 30 min at 65° C. in 2×SSC, 0.1% SDS, followed by two 30-min washes in 1×SSC, 0.1% SDS and 1×SSC, 0.5% SDS. Northern blots were visualised by Phosphorimager.

Soleus muscle preparation. All experiments were performed according to protocols approved by the Institutional Animal Ethics Committee. 5-6 week-old male Wistar rats (160 g) were kept on a 12:12-h light/dark cycle, and fed and watered ad libitum. Animals were anaesthetised with 50 mg/kg Nembutal (pentobarbitone sodium), then sacrificed by cervical dislocation. Soleus muscles were dissected under oxygenated KHB (118 mM NaCl, 4.75 mM KCl, 1.2 mM MgSO₄, 24.8 mM NaHCO₃, 1.2 mM KH₂PO₄, 2.54 mM CaCl₂, 10 mM glucose, pH 7.4) and teased into halves, then incubated as described below.

Determination of cAMP in cultured cells and soleus muscle strips. cAMP concentration was measured in cells seeded in six-well plates and transfected as described. Following aspiration, cells were incubated for 15 min at room temperature in the presence of stated concentrations of ligands in binding buffer. Isobutylmethylxanthine (Sigma) was added to a final concentration of 0.5 mM together with stated concentrations of peptides, and the reaction terminated by aspiration. Cells were washed with 0.5 ml ice-cold TE buffer (10 mM Tris/HCl, 1 mM EDTA, pH 7.6) and rapidly transferred to eppendorf tubes in 1 ml of the same buffer, then boiled for 5 min. Following centrifugation at 12,000 g for 5 min at 4° C., then cooling on ice, duplicate 50 μl aliquots were analyzed for cAMP using an assay system based on competitive binding (Arnersham Pharmacia) according to the manufacturer's instructions.

For the measurement of cAMP in muscle, isolated soleus strips were incubated with stated concentrations of peptides, then lyophilized and deproteinized. Each strip was placed in 400 μl of 0.5 M HClO₄ with 5 mM EDTA (phosphodiesterase inhibitor) and sonicated three times in 20 s bursts. Tubes were heated for 3-5 min in boiling water to coagulate protein, then centrifuged (12,000 g, 4° C., 2 min). Supernatant (300 μl) was taken and the pH adjusted to 7.0 with 75 μl of 2 M NH₄OH. The samples were refrigerated (4° C., 2 h) to precipitate proteins, then centrifuged under the same conditions as above and 50 μl taken for cAMP measurement.

Determination of rates of glucose transport in incubated soleus muscle strips. 4-5 muscle strips were added to each flask, which contained 10 ml DMEM. They were pre-incubated with hormones as indicated for 30 min at 30° C. in a shaking water bath, after which 5 μl of 2[³H]DOG (0.5 μCi/ml) and 5 μl D[¹⁴C(U)]sorbitol (0.05 μCi/ml) were added, then incubated for another 30 min; RIA grade BSA was added to 0.9% (w/v) to prevent non-specific binding of insulin to flasks. Following incubation, muscle strips were transferred to ice-cold KHB for 5-10 min to remove excess [³H]DOG and [¹⁴C]sorbitol. They were then blotted, frozen, tendons excised, then lyophilized for a further 48 h. Muscles were then weighed, transferred to scintillation vials and digested with 0.5 ml 1 M NaOH at 60° C. for 1 h. Scintillation fluid (4 ml) was added to each vial, and samples counted in a Beckman liquid scintillation spectrometer, preset to count ¹⁴C and ³H channels simultaneously. The amount of each isotope present in the samples was determined and used to calculate the extracellular space and the intracellular concentration of [³H]DOG. The accumulation of intracellular [³H]DOG, a measure of muscle glucose uptake, was calculated by subtracting the concentration of [³H]DOG in the extracellular space from the corresponding total muscle [³H]DOG]; extracellular [³H]DOG was quantified by measuring the muscle concentration of [¹⁴C]sorbitol.

Measurement of glycogen metabolism in isolated, incubated soleus muscle. For measurement of incorporation of D[¹⁴C(U)] glucose into muscle glycogen, muscle strips were pre-incubated in DMEM for 30 min, followed by incubation for 2 h with 0.5 μ Ci D[¹⁴C(U)] glucose and amylin, CGRP or sCT at stated concentrations, in the absence or presence of 23.7 nM insulin. Strips were rapidly frozen, freeze-dried, weighed then digested in 60% (w/v) potassium hydroxide (5 mg of dried muscle/0.4 ml 10.7 M potassium hydroxide) for 45 min at 70° C. with occasional mixing. Glycogen was precipitated overnight at −20° C. with 96% ethanol (1.2 ml), followed by centrifugation at 8,000 g (15 min, 4° C.). Pellets were washed twice with the same amount of ethanol then dissolved in water (0.6 ml). Aliquots (0.3 ml) were analyzed for D[¹⁴C(U)] glucose content by liquid scintillation spectrometry.

Total glycogen content was measured in muscle strips by lyophilization followed by hydrolysis of glycogen to D-glucose with amyloglucosidase (Sigma) 20 U/100 ml in 0.2 M sodium acetate/acetic acid buffer, pH 4.8, and expressed as μmol ‘glucosyl’ units/g dry muscle weight. Free glucose in supernatant solutions was measured with a glucose/lactate analyzer (YSI 2300STAT, Yellow Springs Corporation) using D-glucose oxidase immobilized enzyme chemistry.

Data analysis. One way ANOVA, Dunnett's Multiple Comparisons Test, and unpaired Student's t-tests were used to calculate the statistical significance of differences, as appropriate. Dose-response curves were fitted to a sigmoidal dose-response algorithm, as follows: Y=minimum+(maximum−minimum)/(1±10 log ^{(EC}₅₀^{-X}), for 2-DOG-uptake; and by a four parameter logistic model, Y=minimum+(maximum−minimum)/(1+10 log ^{(EC}₅₀^{-X)Hill-Coefficient}) for total glycogen and [¹⁴C]glucose incorporation. Calculations were performed using Prism v2.0 (GraphPad Software, San Diego, Calif.) to derive values for half-maximal effective concentration (EC₅₀). Displacement curves derived for competition of bound radioligand with cold ligand in transfected cells were calculated according to a one site competition fit using Prism v2.0.

### Example 8

### Radioligand Binding to L6 Myoblasts Co-transfected With Murine Ramp and C1ᵢₙₛ₋

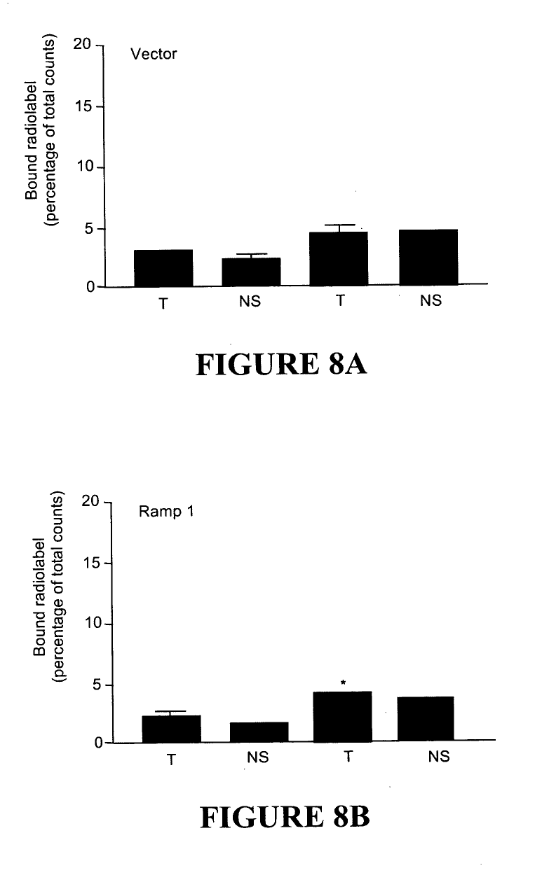

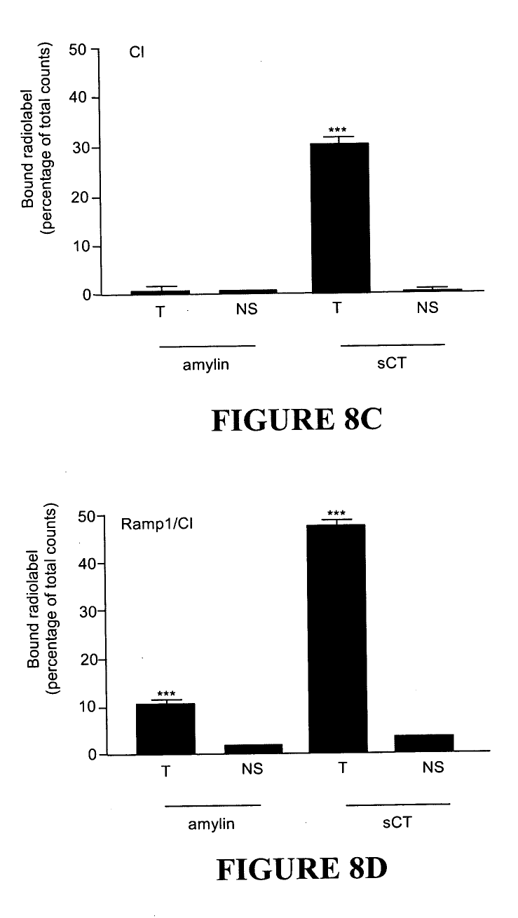

Occurrence of receptors responsive to amylin has previously been reported when the insert-negative isoform of the calcitonin receptor, C1ᵢₙₛ₋, was co-expressed with human isoforms of either ramp 1 or ramp 3 (Muff et al., 1999, Endocrinology 140:2924-2927; Christopoulos et al., 1999, Mol. Pharmacol. 56:235-242). Here, we have measured properties of a corresponding murine receptor isoform that is constituted when murine ramp 1 and murine C1ᵢₙₛ₋ are co-transfected into L6 (murine) myoblasts (FIG. 8). No significant specific binding of amylin was detected in cells transfected with vector alone (FIG. 8A), ramp 1 alone (FIG. 8B), or C1ᵢₙₛ₋ alone (FIG. 8C), whereas sCT did exhibit low but significant specific binding in cells transfected with ramp 1 alone (FIG. 8B). sCT but not amylin showed significant binding to cells transfected with C1ᵢₙₛ₋ alone (FIG. 8C) whereas by contrast, significant increases in specific binding of both amylin and sCT occurred when ramp 1 and C1ᵢₙₛ₋ constructs were co-transfected (FIG. 8D). Time course experiments established the incubation period required for binding of [³H]amylin to reach equilibrium (results not shown).

### Example 9

### Displacement of Bound [³H]Amylin From Transfected L6 Myoblasts

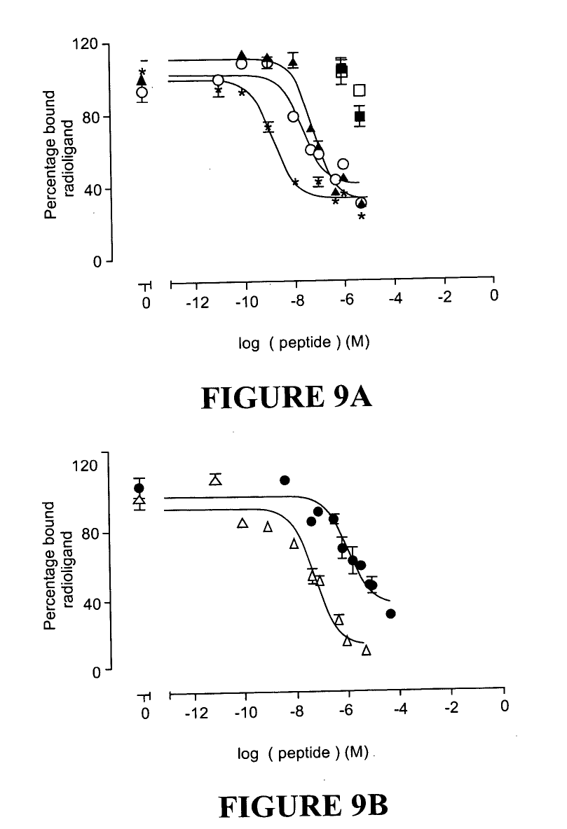

CGRP-1 and amylin displaced specifically-bound [³H]amylin from L6 myoblasts co-transfected with C1ᵢₙₛ₋ and ramp 1, with similar Kₗ and EC₅₀ values (FIG. 9A). This finding is consistent with both peptides competing for binding at a single molecular site on the cell surface. By contrast, sCT had a higher affinity and lower Kₗ, consistent with its higher percentage binding (Table 12; FIGS. 8C and D). Rat calcitonin and human adrenomedullin, a representative of the fifth member of this peptide family, competed only very weakly with amylin for binding to co-transfected cells (FIG. 9A). By contrast to the similar binding characteristics of the full-length peptides, significant differences in binding affinities were observed between the linear, truncated peptides ^{8,37}amylin and ^{8,37}CGRP, which act as antagonists (Table 12; FIG. 9B). ^{8,37}CGRP displayed an approximately 20-fold lower Kₗ for binding to these cells than did ^{8,37}amylin (FIG. 9B). Measured IC₅₀ and derived Kₗ values are summarised in Table 12.

The Kₗ value derived for rat amylin in this study (Table 12) is in close agreement with the K_{d} value derived here from saturation experiments for [³H]amylin (9.2±0.2 nM, mean±S.E.M; Bₘₐₓ=1.5 pmoles/well). These observations are consistent with retention of full functionality in the [³H]methylated ligand.

TABLE 12
BINDING PARAMETERS DERIVED FOR CALCITONIN
FAMILY PEPTIDES/ANTAGONISTS AND THE MURINE
RAMP 1/C1_{INS-} RECEPTOR IN CO-TRANSFECTED
L6 MYOBLASTS.

| | R² | IC₅₀ (nM) | | K₁ (nM) | |
|---|---|---|---|---|---|
| salmon calcitonin | 0.96 | 1.9 | (0.9-2.5) | 0.5 | (0.3-8) |
| CGRP | 0.96 | 63 | (43-93) | 20 | (13-29) |
| amylin | 0.92 | 23 | (13-44) | 7.4 | (4-14) |
| ^{8,37}CGRP | 0.92 | 62 | (35-108) | 19.5 | (11-34) |
| ^{8,37}amylin | 0.87 | 1270 | (661-2420) | 399 | (208-763) |

K₁ values were calculated by the method of Cheng and Prusof (Cheng et al., 1973, Biochem. Pharmacol. 22: 3099-3108) using a radioligand concentration of 20 nM and a K_{d} of 9.2 that was derived independently from a saturation curve. 95% confidence intervals (nM) are bracketed.

### Example 10

### Effect of Peptide Antagonists on CGRP and Amylin-evoked Camp Production in Transfected L6 Myoblasts

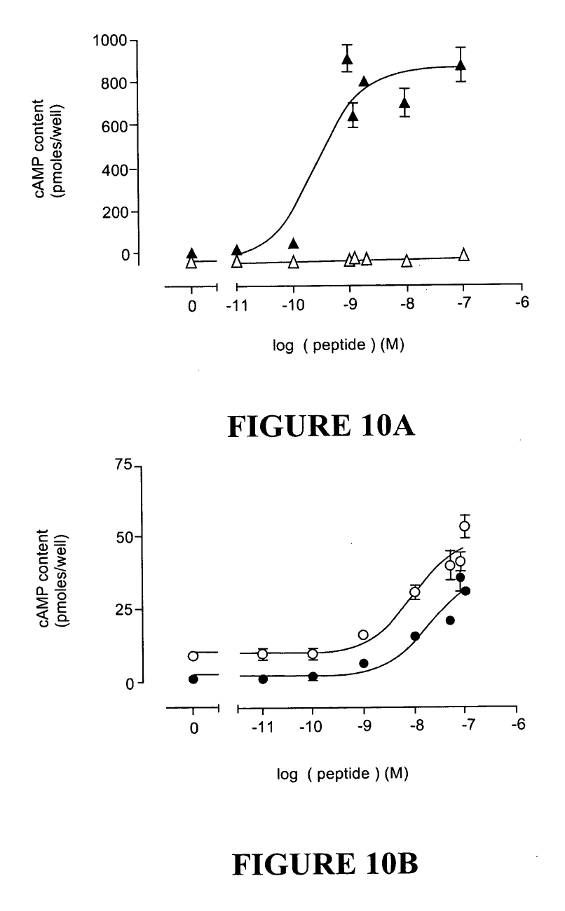

To investigate their roles as antagonists of peptide-evoked signal transduction, effects of ^{8,37}CGRP and ^{8,37}amylin on cAMP-production elicited by CGRP and amylin, were studied for the constituted ramp 1/C1ᵢₙₛ₋ receptor phenotype (FIG. 10). CGRP and amylin both evoked dose-dependent increases of cAMP production in co-transfected myoblasts, although the magnitude of this response was markedly less for amylin at all concentrations studied (FIGS. 10A and B).

5 μM ^{8,37}CGRP abolished cAMP elevation evoked by CGRP, at all concentrations studied (10 pM to 100 nM; FIG. 10A). This concentration is sufficient to saturate the receptor, being approximately 250-fold greater than the Kₗ values measured for CGRP and amylin. Consistent with the similarity between Kₗ values of CGRP and ^{8,37}CGRP, the highest concentration of CGRP applied (100 nM) was competed effectively by a 50-fold excess of ^{8,37}CGRP (5 μM). By contrast, however, 5 μM ^{8,37}amylin was significantly less effective as an antagonist of amylin, evoking only a modest decrease in cAMP production (FIG. 10B).

### Example 11

### Radioligand Binding to Cells Following Transfection With Ramp 3 and C1ᵢₙₛ₋

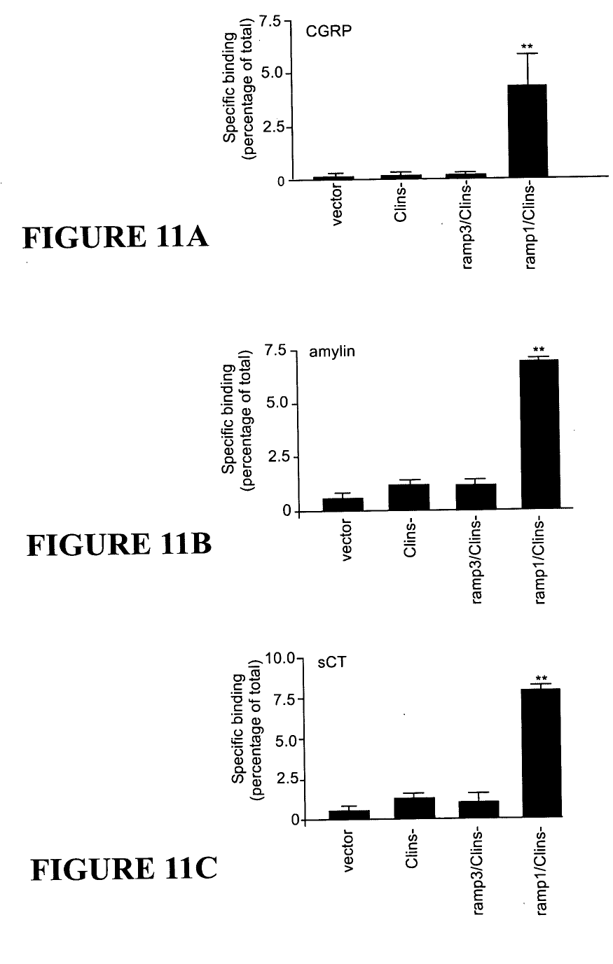

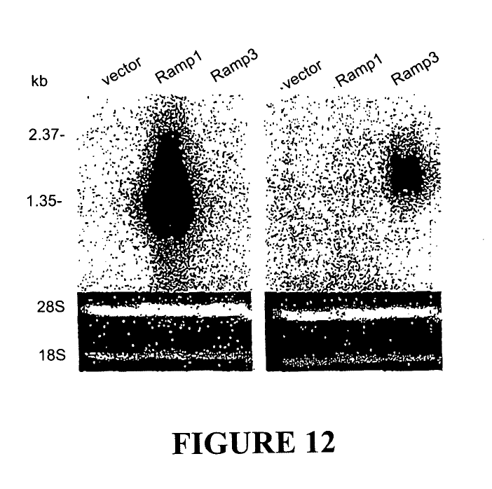

Co-transfection of myoblasts with murine ramp 3 and C1ᵢₙₛ₋ did not yield significant specific binding with any of the radioligands employed (results not shown). Co-transfection of cos-7 cells has previously been reported to give binding of [¹²⁵I]-rat amylin or [125I]-human amylin to both C1ᵢₙₛ₋/ramp 1 and C1ᵢₙₛ₋/ramp 3 human receptor isoforms (Muff et al., 1999, Endocrinology 140:2924-2927; Christopoulos et al., 1999, Mol. Pharmacol. 56:235-242). We therefore performed comparative studies with the corresponding murine isoforms in such cells. Our results for co-transfection of C1ᵢₙₛ₋/ramp 1 were similar to the published results (FIGS. 11A to C). However, consistent with our results in L6 myoblasts, tranfection with murine C1ᵢₙₛ₋/ramp 3 failed to yield specific binding. Transcription from the ramp 3 construct in Cos cells was confirmed by Northern analysis (FIG. 12, right panel), although the transcript was less abundant than that corresponding to ramp 1 (FIG. 12, left panel). However, no change in transcription efficiency from the ramp 3-plasmid was observed in this system, across a range of concentrations (data not shown). Non-transfected cos-7 cells exhibited small but significant endogenous specific binding to all three [³H]peptides, suggesting that they may express low levels of a common receptor. Indeed, RT-PCR revealed that both L6 and Cos-7 cells express C1ᵢₙₛ₋ (data not shown), suggesting that the observed differences in binding affinites could be due to variation in endogenous expression of ramp 1 or ramp 3.

### Example 12

### Effects of CGRP and Amylin on Camp Content of Soleus Muscle

The relationship between CGRP/amylin signaling and cAMP metabolism in skeletal muscle is controversial, due in part to inconsistencies between different published studies. Early work suggested that amylin did not affect skeletal muscle cAMP content, consistent with the existence of a cAMP-independent signaling mechanism (Deems et al., 1991, Biochem. Biophys. Res. Commun. 174:716-720; Kreutter et al., 1993, Am. J. Physiol. 264:E606-613) These findings contrast other reports of potent stimulation by amylin of cAMP and adenylyl cyclase activity in skeletal muscle (Pittner et al., 1995, Biochimica Biophys. Acta 1267:75-82).

a) Nanomolar Concentrations of CGRP and Amylin Elict cAMP Production via the Metabolic Amylin Receptor

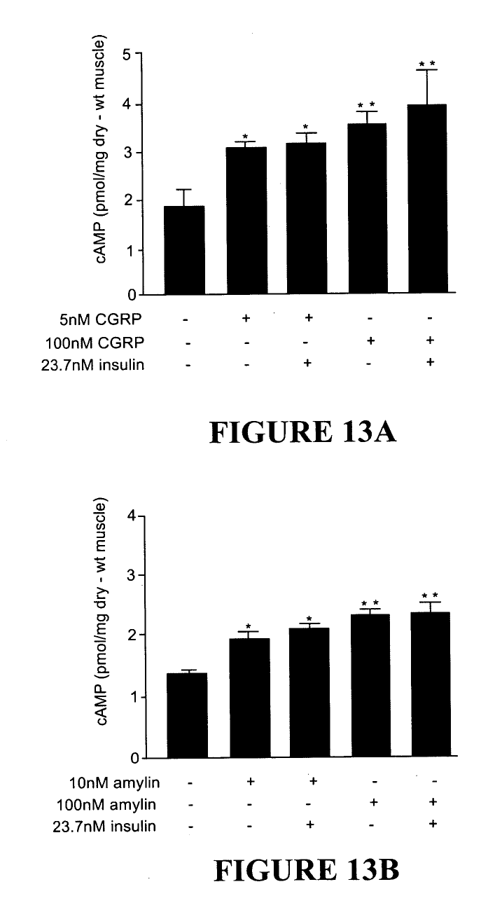

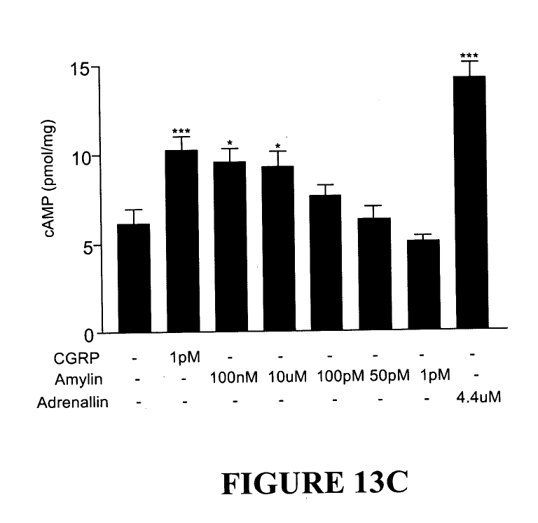

Here, in vitro effects of CGRP (FIG. 13A) and amylin (FIG. 13B) on cAMP production in soleus muscle were measured and compared with their effects on cAMP production in cells co-transfected with C1ᵢₙₛ₋/ramp 1 (FIGS. 10A and B). The effect of amylin on cAMP content in soleus muscle was shown to be maximal by 5 min after addition of amylin (2-fold increase, p<0.01, data not shown), which is comparable with the time course previously reported for the response to isoproterenol (a β-adrenergic agonist) in isolated perfused hearts. CGRP, at both 5 nM and 100 nM, elicited significant increases in muscle cAMP content (FIG. 13A), both in the absence or presence of insulin (23.7 nM). Amylin evoked a maximal increase in cAMP content of about 2-fold when added at either 10 nM or 100 nM (FIG. 13B); the magnitude of this effect was not different in the absence or presence of insulin (23.7 nM). Picomolar concentrations of amylin had no effect on muscle cAMP levels, illustrating a difference in the activity of amylin and CGRP-1 (FIG. 13C).

These biological effects of CGRP and amylin are consistent with their effects to stimulate cAMP content in L6 myoblasts transfected with C1ᵢₙₛ₋/ramp 1 (FIG. 10). Furthermore, the suppression of amylin-mediated cAMP stimulation by the competitive antagonist, ^{8,37}amylin (FIGS. 9B and 11B) is consistent with the view that amylin evokes this effect in physiological muscle through a C1ᵢₙₛ₋/ramp 1 receptor.

b) Picomolar Concentrations of CGRP1 Elict cAMP Production via the High Affinity Receptor

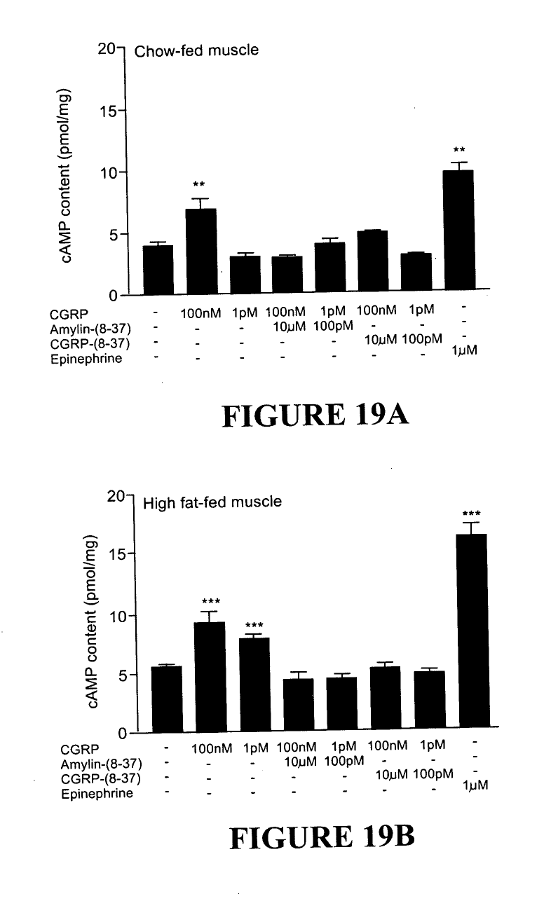

In soleus muscle from normal fed animals, 100 nM CGRP (6.9±0.8 vs. 3.9±0.2 pmol/mg, P<0.01) and adrenaline, (9.6±0.7 vs. 3.9±0.2 pmol/mg, P<0.01) evoked significant increases in cAMP content. A 10-fold molar excess of either CGRP-(8-37) (4.8±0.2 vs. 6.9±0.8 pmol/mg, P<0.01) or amylin-(8-37) (2.9±0.8 pmol/mg, P<0.01) reversed this increase. No significant cAMP increase was observed following 1 hr incubation with 1 pM CGRP. See FIG. 19A.

In soleus muscle isolated from high fat-fed animals, 100 nM CGRP1 (9.3±1 vs. 5.7±0.2 pmol/mg, P<0.001) also evoked significant increases in cAMP content, and which was reversed by 10-fold molar excesses of either amylin-(8-37) (4.3±0.8 pmol/mg; P<0.001) or CGRP-(8-37) (5.4±0.4; P<0.001). However, in contrast to soleus muscle from normal-fed animals, incubation with 1 pM CGRP significantly increased cAMP content (8.6±0.4 vs. 5.7±0.2 pmol/mg, P<0.001). 10-molar excesses of either amylin-(8-37) or CGRP-(8-37) reversed this increase from 8.6±0.4 pmol/mg to 4.4±0.4 pmol/mg (P<0.001) and 4.7±0.5 pmol/mg (P<0.001) respectively. See FIG. 19B.

There were significant differences in basal cAMP content of resting soleus muscle between the normal and high fat-fed diets (5.7±0.2 vs. 3.9±0.2 pmol/mg, P<0.01).

### Example 13

### Effects of Competitive Antagonists on Amylin-mediated Suppression of Glucose Transport

The effects of amylin (10 nM) on basal and insulin-stimulated glucose transport in soleus muscle were measured with 2-DOG after 30 min of incubation, and are reported in Table 13. Treatment with amylin alone did not significantly affect glucose uptake, whereas insulin stimulated glucose uptake by 3.2-fold (p<0.01). By contrast, amylin (10 nM) decreased insulin-stimulated glucose uptake by 31%, to a value not significantly different from basal.

Both ^{8,37}amylin and ^{8,37}CGRP antagonize effects of CGRP and amylin in various tissues (reviewed in Cooper, 2001, in HANDBOOK OF PHYSIOLOGY. SECTION 7: THE ENDOCRINE SYSTEM. VOLUME 11: THE ENDOCRINE PANCREAS AND REGULATION OF METABOLISM (Jefferson, L S Cherrington, A D, eds.), Oxford University Press). Here, soleus muscle strips were preincubated in vitro with amylin (10 nM) and insulin (23.7 nM), plus stated final concentrations of ^{8,37}amylin or of ^{8,37}CGRP (FIG. 14). 8,37 CGRP, at concentrations of both 1 μM and 10 μM, effectively antagonized the inhibitory effect of amylin on insulin-stimulated glucose uptake (FIG. 14A), from 19.5±3.6 nmol/g dry-wt muscle/min (n=5) to 30.4±3.0 (n=4, p<0.05). By contrast, ^{8,37}amylin had no significant effect on amylin-mediated suppression of glucose uptake, even at 10 μM (FIG. 14B).

TABLE 13
EFFECT OF RAT AMYLIN ON RATES OF BASAL (NO
INSULIN) OR MAXIMAL (23.7 NM)-STIMULATED
GLUCOSE UPTAKE, AS MEASURED WITH 2-DEOXY-D-
GLUCOSE (2-DOG), INTO ISOLATED SOLEUS MUSCLE
STRIPS INCUBATED WITH HORMONES

| | | 2-DOG-uptake | | |
|---|---|---|---|---|
| | | (nmol/g dry-wt muscle/min) | | level of significance |
| basal | | 8.8 ± 0.9 | (n = 3) | — |
| amylin | (10 nM) | 13.4 ± 2.4 | (n = 5) | p > 0.05 |
| insulin | (23.7 nM) | 28.4 ± 6.4 | (n = 3) | **p < 0.01 |
| amylin | (10 nM) + | 19.5 ± 3.6 | (n = 5) | p > 0.05 |
| insulin | (23.7 nM) | | | |

Statistical analysis was performed using one-way ANOVA followed by post hoc analysis using Dunnett's Multiple Comparisons Test. Level of significance refers to comparisons between rates in hormone-treated muscle compared with those in the basal condition. n = number of independent experiments.

### Example 14

### Comparison of Effects of CGRP and Amylin on Glycogen Metabolism in Soleus Muscle

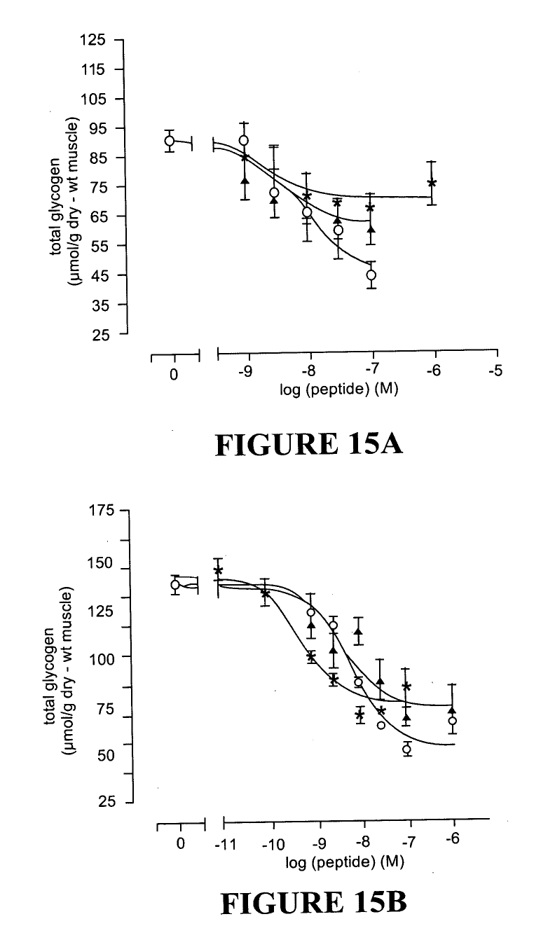

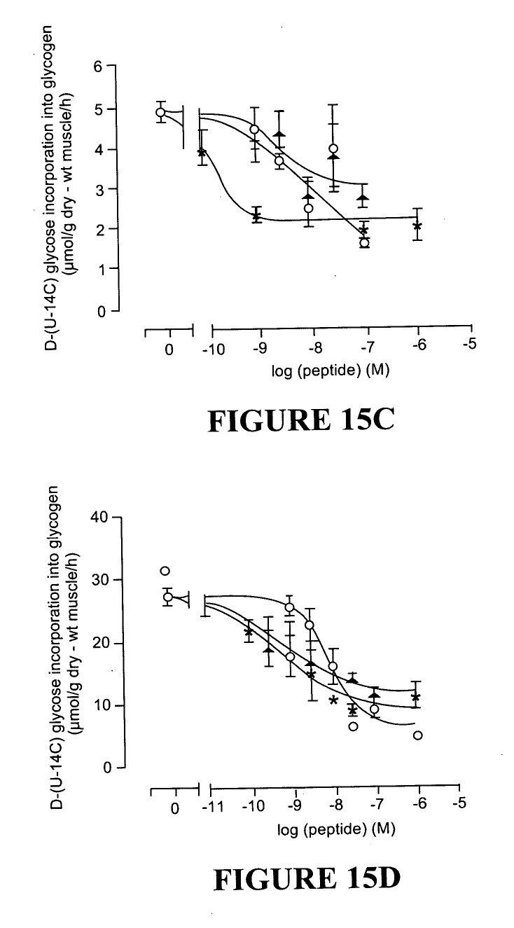

Preliminary experiments established that total glycogen production and incorporation of D[¹⁴C(U)] glucose into glycogen are maximal in isolated soleus muscle at 23.7 nM insulin (results not shown). The effects of increasing concentrations of CGRP, amylin and sCT on total glycogen content were thus measured in the absence or presence of insulin at this concentration following 2 h incubations (FIG. 15). Concentration-response curves for glycogen content, showed that 10 nM amylin elicited significant decrements in total glycogen content (FIG. 15A), from a basal rate of 90.8±3.7 μmol/g dry-wt muscle/h (n=15) to 65.6±9.1 (n=4; p<0.05). A similar decrease in glycogen content was also seen with 10 nM CGRP, to 71.4±7.7 μmol/g dry-wt muscle/h, although this difference failed to reach statistical significance (n=4, p=N.S.). In the presence of insulin, the inhibitory effects of both peptides were more pronounced (FIG. 15B). Treatment with 10 nM amylin decreased total glycogen content from 138±4.8 μmol/g dry-wt muscle/h (n=12) to 86.7 μmol/g dry-wt muscle/h (n=4; p<0.01). By contrast, incubation with 10 nM CGRP decreased glycogen content only to 113±6.9 μmol/g dry-wt muscle/h (p=N.S.). The maximal effect of amylin on muscle glycogen content was therefore more pronounced than that of CGRP. Effects of sCT on glycogen content were not significantly different from those of CGRP (FIGS. 15A and B).

The rate of incorporation of D[¹⁴C(U)] glucose into glycogen is a measure of de novo glycogen synthesis in skeletal muscle. D[¹⁴C(U)] glucose was therefore added to incubation medium together with peptides, and rates of incorporation into glycogen measured in muscle strips following 2 h incubations. Treatment with 10 nM amylin in the absence of insulin decreased rates of glucose incorporation, from 4.8±0.3 μmol/g dry-wt muscle/h (n=16) to 2.7±0.8 (n=5, p<0.01; FIG. 15C). Similarly, 10 nM CGRP evoked decreased glucose incorporation, to 2.8±0.3 (n=7, p<0.01). In the presence of maximal insulin, 10 nM amylin decreased radiolabelled glucose incorporation (FIG. 15D), from 27.6±1.4 (n=11) to 16.0±2.5 (n=4, p<0.05), whereas the equivalent concentration of CGRP suppressed glucose incorporation to 13.7±0.7 μmol/g dry-wt muscle/h (n=4, p<0.01). In the absence of insulin, the response to sCT was significantly more potent than either that of CGRP or of amylin (FIG. 15C), whereas in the presence of insulin, the sCT response was equivalent to that of CGRP (FIG. 15D). The measured decrements in glucose incorporation evoked by all three peptides occurred in the presence of declining muscle glycogen content, and therefore represent decreased rates of glycogen synthesis.

EC₅₀ values and 95% C1 (confidence intervals) were derived from these concentration-response curves (Table 14). In the presence of insulin, amylin was significantly less potent than the other two peptides. Significant differences between peptides with respect to maximal inhibition of glycogen synthesis were observed. These findings indicate that there is some heterogeneity between the receptor sub-types through which the three peptides elicit maximal inhibition of glycogen synthesis in physiological skeletal muscle.

TABLE 14
CALCULATED VALUES FOR EC₅₀ AND 95% CONFIDENCE
INTERVALS (CI) FOR TOTAL GLYCOGEN CONTENT AND RATES
OF ¹⁴C-GLUCOSE INCORPORATION INTO GLYCOGEN
IN ISOLATED RAT SOLEUS MUSCLE INCUBATED IN VITRO

| | | | | | ¹⁴C-glucose incorporation into | | | |
|---|---|---|---|---|---|---|---|---|
| | total glycogen content | | | | glycogen | | | |
| | (μmol/g dry-wt muscle/h) | | | | (μmol/g dry-wt muscle/h) | | | |
| | (−INSULIN) | | (+INSULIN) | | (−INSULIN) | | (+INSULIN) | |
| | EC₅₀ | 95% CI | EC₅₀ | 95% CI | EC₅₀ | 95% CI | EC₅₀ | 95% CI |
| amylin | 8.0 | 1.9-33.0 | 8.1 | 3.5-18.3 | 9.4 | 0.2-476 | 9.2 | 4.0-21.0 |
| CGRP-1 | 3.2 | 0.4-27.2 | 4.3 | 1.2-14.6 | 3.1 | 0.3-27.8 | 0.7 | 0.3-2.1 |
| sCT | 1.7 | 0.01-48.2 | 0.6 | 0.3-1.3 | 0.1 | 0.1-52 | 0.9 | 0.3-2.8 |

| | | | | | | | | |
|---|---|---|---|---|---|---|---|---|
| Difference | p = | N.S. | p = | N.S. | p = | N.S. | p = | 0.01 |
| in EC₅₀ | | | | | | | | |
| Difference | p < | 0.0001 | p < | 0.0001 | p < | 0.0001 | p < | 0.001 |
| in maximal | | | | | | | | |
| inhibition | | | | | | | | |

Rat soleus muscle strips were incubated with amylin, CGRP-1 or sCT in the absence or presence of a maximally effective concentration of human insulin (23.7 nM), as shown in FIG. 15. Significance was determined by two-way ANOVA.

These experiments show that a receptor with the characteristics of the metabolic amylin receptor is constituted by co-transfection of the calcitonin receptor (insert-negative form) [C1ᵢₙₛ₋] with ramp 1 into either L6 myoblasts or cos-7 cells . This receptor constitutes strong specific binding of CGRP, amylin and sCT, and transduction of signals by both CGRP and amylin. By contrast, rat calcitonin and human adrenomedullin, further representatives of the CGRP/amylin peptide family, have much lower binding affinities at this receptor. As is shown in the Examples, infra, the biological effects of these peptides as ligands at the reconstituted receptors was compared with their actions in isolated rat soleus muscle, which is one of their known pharmacological target tissues. Measured binding affinities for peptide ligands at the C1ᵢₙₛ₋/ramp 1 receptor have the same relative quantitative order as that of their respective EC₅₀ values for inhibition of glycogen synthesis in intact skeletal muscle. This agonist order is sCT>CGRP≈amylin>>rat calcitonin, human adrenomedullin. The similar affinities of CGRP and amylin for this receptor are compatible with the similar EC₅₀ values derived for these peptides for their stimulation of cAMP. Tissue distribution studies indicate that C1ᵢₙₛ₋ is the predominant molecule transcribed from calcr in rat skeletal muscle. These results are consistent with CGRP and amylin evoked inhibition of glycogen synthesis by action as agonists at a C1ᵢₙₛ₋/ramp 1 receptor in skeletal muscle. Other CGRP receptor subtypes have been characterized at the molecular level, which are constituted by co-transfection with ramp constructs and either C1ᵢₙₛ₋ or the related G-coupled receptor, calcitonin receptor-like receptor, CRLR. See, McLatchie et al., 1998, Nature 393:333-339; and Buhlmann et al., 1999, Endocrinology 140:2883-2890.)

The action of rat CGRP-1 and amylin as antagonists of insulin action were also carried out in soleus muscle isolated from high fat-fed rats. These experiments show that no change in the potency of CGRP as a non-competitive antagonist of insulin action in high fat-fed muscle, illustrating that the effects on lipid content in high fat-fed muscle are uncoupled from undesirable antagonist effects on insulin-stimulated glucose incorporation into glycogen.

### Example 15

### Effects on Tissue Lipid and Metabolites Following Acute CGRP Infusion (1 hr) in Normal-fed Rats Methods

Surgery. Experiments were performed on male Wistar rats. Animals were randomly assigned to experimental groups as outlined below. Induction and maintenance of surgical anesthesia was by 3-5% halothane and 2L oxygen. An initial blood sample was taken from a tail artery for measurement of baseline levels of blood glucose and lactate, electrolyte, acid base and oxygen status and serum levels of free fatty acids, triglyceride and cholesterol. Immediately following this, the trachea was cannulated and the animal ventilated at 60-70 breaths.min-1 with air supplemented with O₂. The respiratory rate and end-tidal pressure (10-15 cmH₂O) were adjusted to maintain end-tidal CO₂ at 35-40 mmHg. Body temperature was maintained at 37° C. throughout surgery and the experiment by a heating pad. The carotid artery and jugular vein were cannulated with a solid-state blood pressure transducer and a saline filled PE 50 catheter respectively. For the antagonist infusion studies, surgery was similar to above with the alteration being that both the carotid artery and jugular vein were cannulated with saline filled PE 50 catheters. Fluids (saline or hormone dissolved in saline) were delivered to the jugular vein with a Y-connector to join the two lines. The carotid artery line was connected to a blood pressure transducer. Blood samples were taken from this line at time zero (baseline reading), 30 minutes and every 20 minutes until the end of the experiment at 90 minutes.

Infusion. Following surgery and a stabilization period of at least 15min, animals underwent infusion of either saline (control) or CGRP (100 pmol.kg-1.min-1, Bachem) at a rate of 1 ml.h-1 for 60 min. This infusion was supplemented by intravenous administration of 154 mmol/L NaCl solution at a rate of 3.3 ml/kg/hr to replace estimated fluid losses. Blood glucose measurements were taken from tail capillaries at 5-minute intervals throughout the duration of the experiment. At the end of the infusion a terminal blood sample was taken by cardiac puncture and analysed as per the basal sample and also for various metabolites and hormones. Serum and plasma samples were stored at −80° C. until future analysis. Animals were killed by cervical dislocation and harvested tissues (brain, liver, kidney, soleus muscle, extensor digitorum longus muscle, left ventricle, retroperitoneal and epididymal fat pads) snap frozen in liquid N2 and stored at −80° C. until future analysis. For the antagonist studies, the protocols was the same as above with the following changes: Infusion was a combination of antagonist, CGRP or saline. Animals received either antagonist or saline for the first 30 minutes at a rate of 1 ml/kg/h, the second line was then started, containing either CGRP or saline as appropriate, again at a rate of 1 ml/kg/h.

Mean arterial pressure (MAP), heart rate (HR, derived from the MAP waveform) oxygen saturation (Nonin 8600V Pulse Oximeter) and core body temperature were all continuously monitored throughout the experiment using a PowerLab/16s data acquisition module. Calibrated signals were displayed on screen and saved to disc as 2 s averages of each variable.

Results

Infusion of CGRP results in specific effects on lipid content in plasma, liver and soleus muscle. Infusion of CGRP in anaesthetised animals, at a dose of 100 pmol/kg/h, resulted in an increase of plasma CGRP compared to saline infused control animals (6.3±0.8 control vs. 325±75 p; P<0.01) (Table 15). There were no significant changes in concentrations of insulin, amylin, epinephrine, and norepinephrine.

TABLE 15
SERUM HORMONE CONCENTRATIONS
FOLLOWING CGRP INFUSION
Whole blood was removed via cardiac puncture and hormones
quantitated by radioimmunoassay or HPLC. Values are means ±
SEM (n = 10 animals for each value). Statistical significance
was analyzed by student T-test.

| Serum hormone | | |
|---|---|---|
| concentration | | |
| (pM) | Control | CGRP |
| Insulin | 810 ± 103 | 631 ± 142 |
| Amylin | 30 ± 5 | 25 ± 5 |
| CGRP | 6.3 ± 0.8 | 325 ± 75** |
| Epinephrine | 6776 ± 509 | 8321 ± 491 |
| Norepinephrine | 2194 ± 80 | 2734 ± 134 |

**Significant difference compared to control, P < 0.01.

A significant increase in plasma free fatty acid concentrations compared to saline infusion (−0.1 mmol/L control vs. 0.4 mmol/L, P<0.01) was observed with CGRP infusion (Table 16). In contrast, the CGRP antagonist, (CGRP-(8-37), at a dose of 10 nmol/kg/h led to a significant reduction in free fatty acid levels (−0.1 mmol/L control vs. −0.3, P<0.05), demonstrating that the antagonist is able to block the activity of the endogenous CGRP peptide. Over the time course of the infusion, CGRP evoked a decrease in plasma triglyceride (0.2 mmol/L control vs. −0.7 mmol/L, P<0.01), while the antagonist showed the opposite effect, raising triglyceride levels to a significant level during the 90-minute infusion (0.2 mmol/L control vs. 0.9 mmol/L, P<0.01). A combination of both peptides (CGRP-(8-37) for 90 minutes+CGRP for 60 minutes) resulted in no change in either plasma free fatty acid or triglyceride content compared to control levels.

TABLE 16
BLOOD METABOLITE LEVELS FOLLOWING CGRP INFUSION.
Whole blood was removed via cardiac puncture, centrifuged for 15
minutes, 10° C. @ 3,000 rpm, serum removed and stored at −80° C.
until further analysis. Free fatty acid and triglyceride concentrations
were quantitated serum using enzyme-based methodologies. Lactate
was measured in heparnised whole blood by an arterial blood gas
analyser. Values shown are the changes in metabolite concentration
expressed as a ratio of the metabolite concentration at the end of the
infusion period divided by the metabolite concentration at the
commencement of the infusion (n = 7 animals at each point).
Statistical significance was analyzed by student T-test.
Change in [serum] (mmol/L)

| | | | | CGRP-(8-37) + |
|---|---|---|---|---|
| | Control | CGRP | CGRP-(8-37) | CGRP |
| Triglyceride | 0.2 | −0.7** | 0.9** | 0.4 |
| FFA | −0.1 | 0.4** | −0.3* | −0.1 |
| Lactate | 0.3 | 1.7** | 0.02 | 0.4 |

| Serum hormone | | |
|---|---|---|
| concentration | | |
| (pM) | Saline infusion | CGRP infusion |
| Insulin | 810 ± 103 | 631 ± 142 |
| Amylin | 30 ± 5 | 25 ± 5 |
| CGRP | 6.3 ± 0.8 | 325 ± 75** |
| Epinephrine | 6776 ± 509 | 8321 ± 491 |
| Norepinephrine | 2194 ± 80 | 2734 ± 134 |

Significant difference compared to control,
**P < 0.01;
*P < 0.05

Analysis of tissue lipid following infusion of CGRP either in the absence or presence the antagonist, CGRP-(8-37) revealed specific effects in soleus muscle and in liver (Table 17). No significant changes in triglyceride and free fatty acid content occurred in heart, epidydmal adipose, or kidney. CGRP infusion resulted in a signficant elevation in free fatty acid and decrease in triglyceride contents in both soleus and liver. In contrast, infusion with the antagonist alone resulted in the opposite effects on these lipids demonstrating blockade of endogenous CGRP action. There were no significant changes in lipid content following co-infusion of CGRP together with antagonist (Table 17).

TABLE 17
CGRP EVOKES TISSUE-SPECIFIC EFFECTS ON FREE FATTY
ACID AND TRIGLYCERIDE CONTENTS IN SOLEUS MUSCLE
AND LIVER
FFA (A) and triglyceride (B) content, of various organs and tissues,
after 90-minute infusion with CGRP (100 pmol/kg/min-for 1 h),
CGRP-(8-37) (10 nmol/kg/min-90 minutes), a combination of the
two, or saline as appropriate. Organs were excised and total lipid
content determined using an enzyme-based methodology. Values are
shown as means ± SEM (n = 10 animals at each point). Statistical
significance was analysed by one-way ANOVA followed by post-hoc
analysis by Dunnetts Multiple Comparsion Test.

| (μmol/ | | | | CGRP-(8-37 + |
|---|---|---|---|---|
| g) | Control | CGRP | CGRP-(8-37) | CGRP |
| A | | | | |
| Heart | 0.8 ± 0.2 | 0.9 ± 0.1 | 0.9 ± 0.2 | 0.7 ± 0.2 |
| Liver | 0.1 ± 0.01 | 0.7 ± 0.1** | 0.06 ± 0.04 | 0.1 ± 0.01 |
| Epi | 2.1 ± 0.5 | 2.7 ± 0.7 | 2.2 ± 0.6 | 2.0 ± 0.3 |
| Adipose | | | | |
| Soleus | 0.2 ± 0.02 | 0.6 ± 0.07** | 0.1 ± 0.02 | 0.2 ± 0.04 |
| Muscle | | | | |
| Kidney | 0.8 ± 0.1 | 0.9 ± 0.2 | 0.8 ± 0.1 | 0.7 ± 0.2 |
| B | | | | |
| Heart | 2.9 ± 0.4 | 2.6 ± 0.4 | 3.1 ± 0.4 | 3.7 ± 0.5 |
| Liver | 4.3 ± 0.3 | 2.2 ± 0.5** | 5.8 ± 0.4* | 3.7 ± 0.3 |
| Epi | 5.1 ± 1.2 | 5.0 ± 0.9 | 4.9 ± 1 | 5.2 ± 1.3 |
| Adipose | | | | |
| Soleus | 3.7 ± 0.4 | 1.5 ± 0.2** | 4.3 ± 0.4* | 3.6 ± 0.5 |
| Muscle | | | | |
| Kidney | 2.8 ± 0.5 | 2.4 ± 0.4 | 2.5 ± 0.4 | 2.3 ± 0.5 |

Significant difference compared to control:
*P < 0.05;
**P < 0.01,
***P < 0.001.

### Example 16

### A CGRP Infusion Dose of 100 pmoles/kg/h CGRP Evokes Effects on Carbohydrate Metabolism and Vasodilation

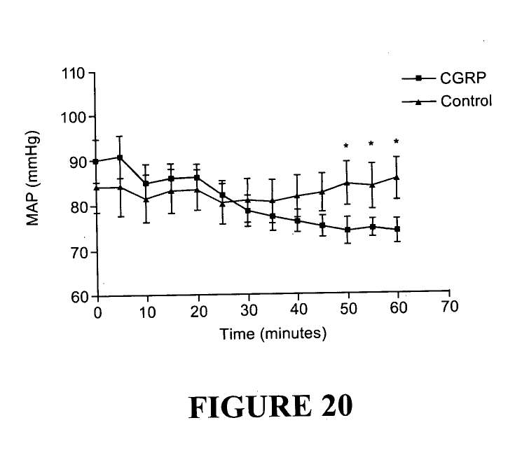

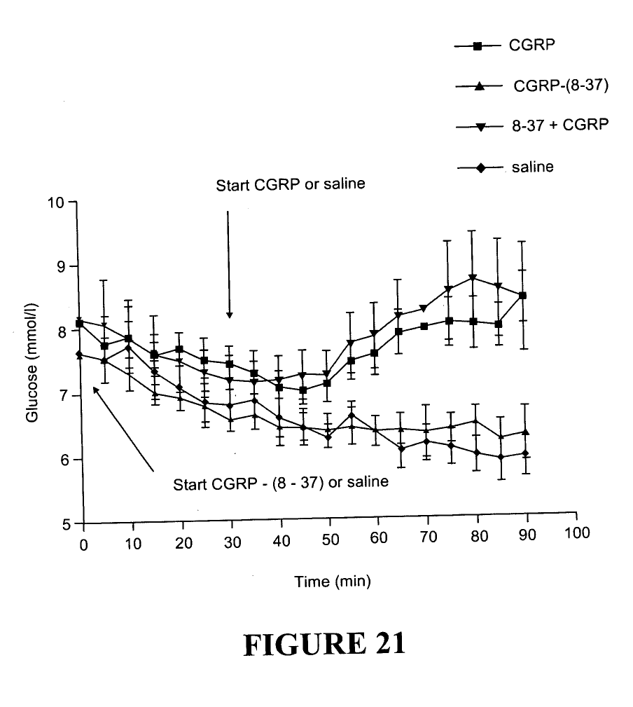

During the 60 minute CGRP infusion described in Example 15, there was a decrease in mean arterial pressure, which reached a significant drop during the final 10 minutes (FIG. 20). Similarly, CGRP infusion also resulted in a significant increase in blood glucose concentration (FIG. 21). A combination of CGRP and its antagonist, CGRP-(8-37), also resulted in increased blood glucose when compared to control animals. Lactate levels were increased during CGRP infusion compared to control levels (0.3 mmol/L vs. 1.7 mmol/L, P<0.01), and again the antagonist showed opposing effects to its agonist, with a small decrease (0.3 mmol/L control vs. 0.02, P<0.05). The antagonist was able to block the increase in glucose concentration by CGRP and with no associated change in lactate levels (0.3 mmol/L saline vs. 0.4 mmol/L, P=NS).

The experiments in Examples 15 and 16 show that a dose of CGRP reached at infusion of 100 pmoles/kg/hr is sufficient to evoke effects on lactate and glucose production, lipid metabolism, and vasodilatation. A CGRP-1 serum concentration of 300 pM was observed in the 100 pmoles/kg/hr infused animals, representing a steady state concentration over the hour infusion period. (Assuming a clearance half-life of CGRP-1 in vivo of 1 0 minutes, this concentration would be reached after 2-3 half-lifes or 20-30 minutes.) Doses resulting in serum concentrations lower than this will be useful for activation of the low affinity receptor without the effects of activation of the metabolic amylin receptor.

### Example 17

### Masoprocol, a Lipooxygenase Inhibitor Reverses CGRP1-Evoked Effects on Muscle Lipid Content in Soleus Muscle

Masoprocol, (nordihydroguaiaretic acid), is a lipoxygenase inhibitor, that has been identified as the active component of Creosote bush extracts. Oral administration of this compound lowers serum NEFA and triglyceride concentrations in rats with fructose-induced hypertriglyceridemia (Gowri et al. 2000, Am. J. Physiol. 279: E593-E600), and decrease blood glucose and triglyceride concentrations in a fat-fed/streptozotocin rat model (Reed et al. 1999, Diabetologia 42: 102-106). Because masoprocol has been shown to exert its antilipolytic actions in adipocytes via inhibition of hormone sensitive lipase (Gowri et al. 2000, Am. J. Physiol. 279: E593-E600), its Effects on CGRP1-evoked effects on NEFA and triglyceride contents in soleus muscle from high fat-fed rats were investigated. Incubation of soleus muscle strips with 50 μM masoprocol alone had no significant effects on either NEFA (74±30 nmol/g basal vs 55.77±21.08 mmol/g) (A) or triglyceride (3.9±0.8 nmol/g basal vs 4.5±0.9 mmol/g) (B) contents. However, masoprocol completely reversed NEFA elevation by 100 nM CGRP1 (533±114 nmol/g; P<0.001) and 1 pM CGRP1 (482±99 nmol/g; P<0.05) nmol/g. Similarly, masoprocol completely reversed decrements in soleus triglyceride content by 100 nM CGRP1 (1.2 mmol/g±0.3 mmol/g; P<0.05) and 1 pM CGRP1 (1.2 mmol/g±0.1 nmol/g; P<0.05).

It is understood that the examples and embodiments described herein are for illustrative purposes only and that various modifications or changes in light thereof will be suggested to persons skilled in the art and are to be included within the spirit and purview of this application and scope of the appended claims. All publications, patents, patent applications, and accession numbers (including both polynucleotide and polypetide sequences and corresponding annotations as of the filing and/or priority application filing dates) cited herein are hereby incorporated by reference in their entirety for all purposes to the same extent as if each individual publication, patent or patent application were specifically and individually indicated to be so incorporated by reference.

What is claimed is:

1. A method of stimulating lipolysis in a tissue of a mammal, comprising contacting the tissue with an agonist of the high affinity CGRP receptor, wherein the tissue is skeletal muscle or liver, and wherein the tissue is contacted with an amount of agonist effective to preferentially stimulate activity of a high affinity CGRP receptor compared to the metabolic amylin receptor.

2. The method of claim 1 wherein the agonist is CGRP-1.

3. The method of claim 2 wherein the amount of CGRP-1 is between about 10⁻¹⁵ M and about 10⁻¹⁰ M.

4. The method of claim 2 wherein the amount of CGRP-1 is less than 300 pM.

5. The method of claim 1 further comprising the step of detecting stimulation of lipolysis in the tissue.

6. A method of reducing skeletal muscle lipid levels in a mammal in need of such treatment, comprising administering to the mammal an agonist of the high affinity CGRP receptor in an amount effective to preferentially stimulate activity of a high affinity CGRP receptor compared to the metabolic amylin receptor.

7. The method of claim 6 wherein the mammal is insulin resistant.

8. The method of claim 7 further comprising the step of detecting a change in the amount of free fatty acids in a tissue of the mammal.

9. The method of claim 8, wherein the tissue is skeletal muscle or blood.

10. The method of claim 6, wherein said administration does not result in an increase in blood glucose levels in the mammal.

11. A method for determining whether an agent is useful for stimulating lipolysis in a mammal, comprising determining whether the agent is an agonist of the high affinity CGRP receptor.

12. The method of claim 11 further comprising determining that the agent preferentially stimulates the high affinity CGRP receptor compared to the metabolic amylin receptor.

13. The method of claim 12 further comprising comparing the EC₅₀ of the agent for effecting a response mediated by the metabolic amylin receptor and the EC₅₀ of the agent for effecting a response mediated by the high affinity CGRP receptor.

14. The method of claim 13, wherein the EC₅₀ values are determined in vitro using isolated skeletal muscle.

15. The method of claim 13, wherein the response mediated by the high affinity receptor is an increase in free fatty acids in skeletal muscle tissue or a skeletal muscle cell and the response mediated by the metabolic amylin receptor is an effect on carbohydrate metabolism.

16. A method of determining whether an agent is useful as a therapeutic agent, comprising
i) determining whether an agent stimulates lipolysis in a mammalian tissue that expresses the high affinity CGRP receptor and the metabolic amylin receptor; and,
ii) determining whether an agent from (i) preferentially stimulates the high affinity CGRP receptor compared to the metabolic amylin receptor,
wherein an agent that preferentially stimulates the high affinity CGRP receptor is determined to be useful as a therapeutic agent.

17. The method of claim 16 wherein the tissue is skeletal muscle.

18. A drug screening method comprising carrying out the method of claim 16 simultaneously or sequentially with at least 25 different agents.

19. A method of determining a dose or formulation of an agonist of the high affinity CGRP receptor that stimulates lipolysis in skeletal muscle of a mammal without eliciting an undesired side-effect in the mammal, said undesirable side-effect being an increased level of blood glucose, blood lactate, or vasodilatation, comprising
i) conducting dose-response assays by
a) administering a plurality of different doses or formulations of the CGRP receptor agonist to test mammals;
b) measuring the effect of each dose or formulation on lipolysis in a tissue of the test mammal and measuring the effect of each dose on the side-effect, thereby creating dose-response data for lipolysis and the side-effect; and,
ii) determining from the dose-response data a dose of the CGRP receptor agonist formulation that stimulates lipolysis but does not elicit the side-effect.

20. The method of claim 19 wherein effect the of each dose or formulation on lipolysis is determined by measuring free fatty acid levels in a tissue of the animal.

21. A pharmaceutical composition in unit dosage form for administration to a mammal, said unit dosage comprising an agonist of the high affinity CGRP receptor in an amount sufficient result in a level of agonist in blood sufficient to preferentially stimulate activity of the high affinity CGRP receptor compared to the metabolic amylin receptor and a pharmaceutically acceptable excipient.

22. The pharmaceutical composition in unit dosage form of claim 21, wherein the agonist is CGRP or a biologically functional variant thereof.

23. The pharmaceutical composition in unit dosage form of claim 22, wherein the agonist is CGRP-1 and the level of agonist in blood is less than 300 pM.

24. The pharmaceutical composition in unit dosage form of claim 22, wherein the agonist is CGRP-1 and the level of agonist in blood is between about 10⁻¹⁵ M and about 10⁻¹⁰ M.

25. A method of stimulating lipolysis in skeletal muscle or liver of a mammal, comprising contacting the skeletal muscle or liver with an agent that is an agonist of the high affinity CGRP receptor and/or the metabolic amylin receptor and monitoring lipolysis in the mammal.

26. The method of claim 25 wherein the agonist is CGRP.

27. The method of claim 25 wherein the amount or composition of free fatty acids in a tissue of the mammal is detected.

28. A therapeutic regimen comprising (i) administering, to a mammal suffering from or susceptible to a condition characterized by accumulation of lipid in skeletal muscle, a CGRP-1 polypeptide, a biologically function variant thereof, or a metabolic receptor stimulating variant thereof and (ii) monitoring lipolysis in the mammal.

29. The therapeutic regimen of claim 28 wherein the monitoring comprises detecting the amount of free fatty acids in a tissue of the mammal.

30. A method of inhibiting lipolysis in skeletal muscle or liver of a mammal, comprising contacting the skeletal muscle or liver with an antagonist of the metabolic amylin receptor and/or high affinity CGRP receptor in an amount sufficient to inhibit lipolysis.

31. The method of claim 30 wherein the antagonist is ^{8,37}amylin or ^{8,37}CGRP.

32. A method of stimulating lipolysis in a tissue of a mammal, comprising contacting the tissue with an agonist of the metabolic amylin receptor.

33. The method of claim 32 wherein the tissue is skeletal muscle.

34. The method of claim 32 wherein the agonist is CGRP.


SEQUENCE LISTING (15 sequences)

SEQ ID NO: 1 - protein, 37 aa, Rattus sp
KCNTATCATQRLANFLVRSSNNLGPVLPPTNVGSNTY

SEQ ID NO: 2 - protein, 37 aa, Rattus sp
SCNTATCVTHRLAGLLSRSGGVVKDNFVPTNVGCEAF

SEQ ID NO: 3 - protein, 30 aa, Rattus sp
KCNTATCATQRLANFLVRSSNNLGPVLPPT

SEQ ID NO: 4 - protein, 30 aa, Homo sapiens
ACDTATCVTHRLAGLLSRSGGVVKNNFVPT

SEQ ID NO: 5 - protein, 37 aa, Homo sapiens
ACDTATCVTHRLAGLLSRSGGVVKNNFVPTNVGSKAF

SEQ ID NO: 6 - protein, 37 aa, Homo sapiens
ACNTATCVTHRLAGLLSRSGGMVKSNFVPTNVGSKAF

SEQ ID NO: 7 - protein, 37 aa, Rattus sp
SCNTATCVTHRLAGLLSRSGGVVKDNFVPTNVGSEAF

SEQ ID NO: 8 - protein, 37 aa, Rattus sp
SCNTATCVTHRLAGLLSRSGGVVKDNFVPTNVGSKAF

SEQ ID NO: 9 - protein, 37 aa, Artificial
  Consensus
XCXTATCVTHRLAXXLXRSGGXXXXNFVPTXVGXXAF

SEQ ID NO: 10 - DNA, 36 bp, Artificial
  Made in Lab
ttaggatccgttgccatggcccggctgcggctcccg

SEQ ID NO: 11 - DNA, 29 bp, Artificial
  Made in Lab
cgagaattcctcatcacccgggataccta

SEQ ID NO: 12 - DNA, 29 bp, Artificial
  Made in Lab
ataggatcctgcatcttagttggccatga

SEQ ID NO: 13 - DNA, 28 bp, Artificial
  Made in Lab
atagaattcatccagcagatcctcaagc

SEQ ID NO: 14 - DNA, 20 bp, Artificial
  Made in Lab
ccatccctgcctgcagatgc

SEQ ID NO: 15 - DNA, 22 bp, Artificial
  Made in Lab
ctctatttctagactgactccc